(19) United States
(12) Patent Application Publication
(10) Pub. No.: US 2006/0079936 A1
(43) Pub. Date: Apr. 13, 2006

(54) Method and system for altering regional cerebral blood flow (rCBF) by providing complex and/or rectangular electrical pulses to vagus nerve(s), to provide therapy for depression and other medical disorders

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/251,492

(22) Filed: Oct. 14, 2005

Related U.S. Application Data
(63) Continuation-in-part of application No. 10/436,017, filed on May 11, 2003.

(51) Int. Cl. A61N 1/00

(52) U.S. Cl. 607/2

(57) ABSTRACT

A method and system for altering regional cerebral blood flow (rCBF) by providing complex and/or rectangular electrical pulses to vagus nerve(s), to provide therapy for depression and other central nervous system (CNS) disorders. Complex electrical pulses comprises pulses which are configured to be one of non-rectangular, multi-level, biphasic, or pulses with varying amplitude during the pulse. The electrical pulses to vagus nerve(s) may be stimulating and/or blocking. The stimulation and/or blocking to vagus nerve(s) may be provided using one of the following pulse generation means: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a microstimulator; e) a programmable implantable pulse generator; f) a combination implantable device comprising both a stimulus-receiver and a programmable implantable pulse generator (IPG); and g) an implantable pulse generator (IPG) comprising a rechargeable battery. The pulse generator means comprises predetermined/pre-packaged programs. In one embodiment, the pulse generation means may also comprise telemetry means, for remote interrogation and/or programming of said pulse generation means utilizing a wide area network.

This application is a continuation of application Ser. No. 10/436,017 filed May 11, 2003, entitled “METHOD AND SYSTEM FOR PROVIDING PULSED ELECTRICAL STIMULATION TO A CRANIAL NERVE OF A PATIENT TO PROVIDE THERAPY FOR NEUROLOGICAL AND NEUROPSYCHIATRIC DISORDERS”.

### FIELD OF INVENTION

The present invention relates to neuromodulation, more specifically to a method for altering regional cerebral blood flow (rCBF) and/or altering neurochemicals in the brain by providing complex and/or rectangular electrical pulses to vagus nerve(s) to provide therapy for depression and other central nervous system (CNS) disorders.

### BACKGROUND

Depression is a significant health issue in the U.S., which has been extensively studied in terms of regional blood flow changes in the brain, and in terms of neurochemicals which are related to depression such as serotonin (5-HT) and norepinephrine (NE).

Regarding blood flow in the brain, a review of clinical studies reveals that patients with major depression have reduced blood flow and glucose metabolism in the prefrontal cortex, anterior cingulate cortex and caudate nucleus when scanned in the resting state and during stressful tests. Apparently, most of these abnormalities are normalized when the patient is cured from the depression. In terms of norepinephrine (NE) and serotonin (5-HT), clinical data shows that both noradrenergic and sertonergic systems are involved in antidepressant action, but the cause of depression is more complex than just an alteration in the levels of serotonin (5-HT) and norepinephrine (NE).

Experimental studies have indicated that afferent vagus nerve stimulation alters regional cerebral blood flow (rCBF) by increasing cerebral blood flow to certain areas of the brain, and decreasing cerebral blood flow to other areas of the brain. Although afferent vagus nerve stimulation has a very different mechanism of action, it reveals similarities in changes of rCBF to those associated with pharmacological treatment, in particular increase of rCBF to the middle frontal gyrus, and a reduction of rCBF in the limbic system and associated regions. Another important process that happens with afferent vagus nerve stimulation is an increase in release of neurochemicals namely serotonin, norepinephrine, and epinephrine. The effect of release of these chemicals is anti-depressant, as well as, anti-epileptogenic.

This patent disclosure is directed to methods of afferent vagus nerve stimulation with complex and/or rectangular electrical pulses to alter regional cerebral blood flow (rCBF), and/or increase the release of serotonin and norepinephrine in the brain to provide therapy or alleviate symptoms of depression. In this disclosure, depression comprises bipolar depression, unipolar depression, severe depression, suicidal depression, psychotic depression, endogenous depression, treatment resistant depression, and melancholia.

### Background of Depression

Depression is a very common disorder that is often chronic or recurrent in nature. It is associated with significant adverse consequences for the patient, patient's family, and society. Among the consequences of depression are functional impairment, impaired family and social relationships, increased mortality from suicide and comorbid medical disorders, and patient and societal financial burdens. Depression is the fourth leading cause of worldwide disability and is expected to become the second leading cause by 2020.

Among the currently available treatment modalities include, pharmacotherapy with antidepressant drugs (ADDs), specific forms of psychotherapy, and electroconvulsive therapy (ECT). ADDs are the usual first line treatment for depression. Commonly the initial drug selected is a selective serotonin reuptake inhibitor (SSRI) such as fluoxetine (Prozac), or another of the newer ADDs such as venlafaxine (Effexor).

Several forms of psychotherapy are used to treat depression. Among these, there is good evidence for the efficacy of cognitive behavior therapy and interpersonal therapy, but these treatments are used less often than are ADDs. Phototherapy is an additional treatment option that may be appropriate monotherapy for mild cases of depression that exhibit a marked seasonal pattern

Many patients do not respond to initial antidepressant treatment. Furthermore, many treatments used for patients who do not respond at all, or only respond partially to the first or second attempt at antidepressant therapy are poorly tolerated and/or are associated with significant toxicity. For example, tricyclic antidepressant drugs often cause anticholinergic effects and weight gain leading to premature discontinuation of therapy, and they can by lethal in overdose (a significant problem in depressed patients). Lithium is the augmentation strategy with the best published evidence of efficacy (although there are few published studies documenting long-term effectiveness), but lithium has a narrow therapeutic index that makes it difficult to administer; among the risks associated with lithium are renal and thyroid toxicity. Monoamine oxidase inhibitors are prone to produce an interaction with certain common foods that results in hypertensive crises. Even selective serotonin reuptake inhibitors can rarely produce fatal reaction in the form of a serotonin syndrome.

Afferent vagus nerve stimulation would provide a device based adjunct (add-on) therapy for patients who do not respond well to initial drug therapy.

### Vagus Nerve Anatomy, Physiology and Mechanisms

The vagus nerves is the tenth cranial nerve in the body, and the only cranial nerves to extend beyond head and neck region into thorax and abdomen. The origin of the vagus nerve in the CNS is the medulla. The vagus nerve carries somatic and visceral afferents and efferents, whose fibers originate mainly from neurons located in the medulla oblongata and in two parasympathetic ganglia. FIG. 1 depicts an overall diagram of the brain, and FIG. 2 depicts the relationship of the vagus nerve(s) 54 to the spinal cord 26, solitary tract nucleus 14, and the overall brain structure.

In the vagus nerve(s), narrow-caliber, unmyelinated C-fibers predominate over faster-conducting, myelinated, intermediate-caliber B-fibers and thicker A-fibers. Neurons of the dorsal motor nucleus of the vagus and the nucleus ambigus provide the efferent axons of the vagus nerve. Vagal efferents innervate striated muscles of the pharynx and larynx, and most of the thoracoabdominal viscera. Afferents (sensory) compose about 80% of the fibers in the cervical portion of the vagus nerve, and efferents (motor) compose approximately 20% of the fibers. A small group of vagal somatsensory afferents carry sensory information from skin on and near the ear. A larger group of special and general visceral afferents carry gustatory information, visceral sensory information, and other peripheral information. Most of the neurons that contributre afferent fibers to the cervical vagus have cell bodies located in the superior (jugular) vagal ganglion and the larger inferior (nodose) vagal ganglion.

The vagus nerve is attached by multiple rootlets to the medulla. The vagus nerve exits the skull through the jugular foramen. In the neck, the vagus nerve lies within the carotid sheath, between the carotid. artery and the jugular vein. In the upper chest, the vagi run on the right and left sides of the trachea. The complex course of the vagi throughout the abdominal and pelvic viscera earned the vagus nerve its name as the Latin term for “wanderer”.

The vagal anatomical pathways of particular relevance to this patent disclosure is that the vagal afferents traverse the brainstem in the solitary tract, terminating with synapses located mainly in the nuclei of the dorsal medullary complex of the vagus. Most vagal afferents synapse in various structures of the medulla. Among these structures, the solitary tract nucleus (NTS) receives the greatest number of vagal afferent synapses, and each vagus nerve synapses bilaterally on the NTS. The vagal afferents carry information concerning visceral sensation, somatic sensation, and taste.

Shown in conjunction with FIG. 3, each vagus nerve bifurcates within the medulla, to synapse bilaterally on the NTS. The NTS is a bilateral pair of small nuclei located in the dorsal medullary complex of the vagus. The NTS extends as a tube-like structure above and below this level within the medulla and caudal pons, as is also shown in FIGS. 22, and 24. The white matter of the tractus solitarius lies in the center of this gray-matter tube, which consists of the multiple subnuclei of the NTS. In addition to dense innervation by the vagus nerves 54, the NTS also receives projections from a very wide range of peripheral and central sources. Also shown in conjunction with FIG. 3, the NTS projects most densely to the parabrachial nucleus of the pons, with different portions of the NTS projecting specifically to different subnuclei of the parabrachial nucleus.

The NTS projects to a wide variety of structures within the posterior fossa, including all of the other nuclei of the dorsal medullary complex, the parabrachial nucleus and other pontine nuclei, and the vermis and inferior portions of the cerebellar hemispheres. The NTS has been likened to a small brain within the larger brain. The NTS receives a wide range of somatic and visceral sensory afferents, and receives a wide range of projections from other brain regions, performs extensive information processing internally, and produces motor and autonomic efferent outputs. The NTS has highly complex intrinsic excitatory and inhibitory connections among its interneurons.

The vagal nerve afferents have widespread projections to cerebral structures mostly using three or more synapses. The NTS projects to several structures within the cerebral hemispheres, including hypothalamic nuclei (the periventricular nucleus, lateral hypothalamic area, and other nuclei), thalamic nuclei (including the ventral posteromedial nucleus, paraventricular nucleus and other nuclei), the central nucleus of the amygdala, the bed of nucleus of the stria terminalis, and the nucleus accumbens. This is also depicted schematically in FIG. 4. Through these projections, the NTS can directly influence activities of extrapyramidal motor systems, ascending visceral sensory pathways, and higher autonomic systems. Through its projections to the amygdala, the NTS gains access to amygdala-hippocampus-entrohinal cortex pathways of the limbic system.

The vagus-NTS-parabrachial pathways support additional higher cerebral influences of vagal afferents, as shown schematically in FIG. 3. The parabrachial nucleus projects to several structures within the cerebral hemipheres, including the hypothalamus (particularly the lateral hypothalamic area), the thalamus (particularly intralaminar nuclei and the parvicellular portion of the ventral posteromedial nucleus), the amygdata (particularly the central nucleus of the amygdala, but also basolateral and other amygdalar nuclei), the anterior insula, and infralimbic cortes, lateral prefrontal cortex, and other cortical regions. The anterior insula constitutes the primary gustatory cortex. Higher-order projections of the anterior insula are particularly dense in inferior and inferolateral frontal cortex of the limbic system. The parabrachial nucleus functions as a major autonomic relay and processing site for autonomic and gustatory information.

The medial reticular formation of the medulla receives afferent projections from the vagus, other cranial nerves, anterolateral tracts of the spinal cord, the substantia nigra, fastigial and dentate nuclei of the cerebellum, the globus pallidus, and widespread areas of cerebral cortex.

Vagal afferents also have access to two special neuromodulatory systems for the brain and spinal cord, via bulbar noradrenergic and serotonergic projections. The locus coeruleus is a collection of dorsal pontine neurons that provide extremely widespread noradrenergic innervation of the entire cortex, diencephion and many other brain structures. Most afferents to the locus coeruleus arise from two medullary nuclei, the nucleus paragigantocellularis and the nucleus prepositus hypoglossi. The NTS projects to the locus coeruleus through two major disynaptic pathways, one via the nucleus paragigantocellularis and the other via the nucleus prepositus hypoglossi.

Vagal-locus coeruleus and vagal-raphe interaction are potentially relevant to VNS mechanisms, since the locus coeruleus is the major source of norepinephrine, and the raphe is the major source of serotonin in most of the brain. Norepinephrine and serotonin exert anti-depressant and anti-seizure effects, in addition to modulating normal thalamic and cortical activities.

Vagal physiology is central to integration of the brain with the periphery in multiple activities of the autonomic and limbic systems, the thalamus, insular cortex, the amygdala, and frontal cortex interact extensively in acute and chronic stress reactions, anxiety, arousal, and reactivity.

The effects of vagus nerve stimulation on brain activation and regional cerebral blood flow have been studied using various imaging techniques. Magnetic resonance spectroscopy (MRS), functional magnetic resonance imaging (fMRI), positron emission tomography (PET), and single photon emission computed tomography (SPECT) permit non-invasive, regional brain mapping of blood flow, glucose metabolism, neurotransmitter concentrations, neurorecptor availability, and other functions. Among these techniques, mapping of regional cerebral blood flow (rCBF) with PET has been employed extensively to study VNS. Relative or absolute regional cerebral blood flow (rCBF) measurements can be made using fMRI, PET, or SPECT. Rapidly occurring changes in regional brain blood flow are considered to primarily reflect changes in trans-synaptic neurotransmission.

In one functional imaging study of acute VNS effects in humans which was reported where stimulation was applied to the vagus nerve during the stimulator-on PET acquisitions. The two groups differed only in the power of stimulation applied to the vagus nerve. Acute VNS induced bilateral rCBF increases in the thalami, hypothalami, and insular and inferior frontal regions, but induced bilateral rCBF decreases in the amygdalae, posterior hippocampi and cingulate gyri. It was concluded that left cervical VNS acutely alters synaptic activities in a widespread and bilateral distribution over brain structures that receive polysynaptic projections from the left vagus nerve.

In summery, the left cervical vagus nerve synapses bilaterally upon the nucleus of the tractus solitarius, the medullary reticular formation, and other medullary nuclei. The nucleus of the tractus solitarius projects densely upon the parabrachial nucleus of the pons, which itself projects heavily to multiple thalamic nuclei, the amygdala, the insula and other cerebral structures. The nucleus of the tractus solitarius projects monosynaptically to several cerebellar sites, monosyaptically to the raphe nuclei (which provide serotonergic innervation of virtually the entire neuraxis), and disynaptically to the locus coeruleus (which provides noradrenergic innervation of virtually the entire neuraxis).

Therapeutic VNS induces widespread bilateral subcortical and cortical alteration of synaptic activity in humans. These VNS-induced alteration in synaptic activity are consistent with known anatomical pathways of central vagal projection. Higher-power VNS causes larger volumes of alteration in cerebral synaptic activities, when comparing groups with high or low levels of VNS.

The vagal afferents have a high degree of access to the major sites of higher processing for the central autonomic network, the reticular activating system (RAS), and the limbic system. The RAS and limbic system are relevant to this disclosure and are as follows.

The limbic system is a group of structures located on the medial aspect of each cerebral hemisphere and diencephalon. Its cerebral structures encircle the upper part of the brain stem, as is shown in conjunction with FIGS. 5A and 5B, which are lateral views of the brain, showing some of the structures that constitute the limbic system. The limbic system include parts of the rhinencephalon (the septal nuclei, cingulate gyrus, parahippocampal gyrus, dentate gyrus, C-shaped hippocampus), and part of the amygdala. In the diencephalon, the main limbic structures are the hypothalamus and the anterior nucleus of the thalamus. The fornix and other fiber tracts link these limbic system regions together.

The limbic system is the emotional or affective (feeling) brain, and is therefore relevant to this disclosure. Two parts that are especially important in emotions are the amygdala and the anterior part of the cingulate gyrus. The amygdala recognizes angry or fearful facial expressions, assesses danger, and elicits the fear response. The cingulate gyrus plays a role in expressing out emotions through gestures and resolves mental conflicts when we are frustrated.

Extensive connections between the limbic system and lower and higher brain regions allow the system to integrate and respond to a wide variety of environmental stimuli. Most limbic system output is relayed through the hypothalamus, which is the neural clearinghouse for both autonomic (visceral) function and emotional response The limbic system also interacts with the prefrontal lobes, so there is an intimate relationship between our feelings (mediated by the emotional brain) and our thoughts (mediated by the cognitive brain). Particular limbic structures, —the hippocampal structures and amygdala—also play an important role in converting new information into long-term memories.

The reticular formation extends the length of the brain stem, as depicted in FIG. 6. A portion of this formation, the reticular activating system (RAS), maintains alert wakefulness of the cerebral cortex. Ascending arrows in FIG. 6 indicate input of sensory systems to the RAS, and then reticular output via thalamic relays to the cerebral cortex. Other reticular nuclei are involved in the coordination of muscle activity. Their output is indicated by the arrow descending the brain stem.

It has been shown that VNS acutely induces rCBF alteration at sites that receive vagal afferents and higher-order projections, including dorsal medulla, somatosensory cortex (contralateral to stimulation), thalamus and cerebellum bilaterally, and several limbic structures (including hippocampus and amygdala bilaterally). The projections of the nucleus of the solitary tract are summarized in FIG. 4.

FIG. 7 shows the effects of vagus nerve stimulation on brain activation and cerebral blood flow using functional magnetic resonance (fMRI) as published by Narayanan et al. in 2002. The curve represents the sum of all activated voxels over the entire brain that are imaged. More actual clinical studies are summarized later in this disclosure.

### Background of Neuromodulation

One of the fundamental features of the nervous system is its ability to generate and conduct electrical impulses. Most nerves in the human body are composed of thousands of fibers of different sizes. This is shown schematically in FIG. 8. The different sizes of nerve fibers, which carry signals to and from the brain, are designated by groups A, B, and C. The vagus nerve, for example, may have approximately 100,000 fibers of the three different types, each carrying signals. Each axon or fiber of that nerve conducts only in one direction, in normal circumstances. In the vagus nerve sensory fibers (afferent) outnumber parasympathetic fibers four to one.

In a cross section of peripheral nerve it is seen that the diameter of individual fibers vary substantially, as is also shown schematically in FIG. 9. The largest nerve fibers are approximately 20 μm in diameter and are heavily myelinated (i.e., have a myelin sheath, constituting a substance largely composed of fat), whereas the smallest nerve fibers are less than 1 μm in diameter and are unmyelinated.

The diameters of group A and group B fibers include the thickness of the myelin sheaths. Group A is further subdivided into alpha, beta, gamma, and delta fibers in decreasing order of size. There is some overlapping of the diameters of the A, B, and C groups because physiological properties, especially in the form of the action potential, are taken into consideration when defining the groups. The smallest fibers (group C) are unmyelinated and have the slowest conduction rate, whereas the myelinated fibers of group B and group A exhibit rates of conduction that progressively increase with diameter.

Nerve cells have membranes that are composed of lipids and proteins (shown schematically in FIGS. 10A and 10B), and have unique properties of excitability such that an adequate disturbance of the cell's resting potential can trigger a sudden change in the membrane conductance. Under resting conditions, the inside of the nerve cell is approximately −90 mV relative to the outside. The electrical signaling capabilities of neurons are based on ionic concentration gradients between the intracellular and extracellular compartments. The cell membrane is a complex of a bilayer of lipid molecules with an assortment of protein molecules embedded in it (FIG. 10A), separating these two compartments. Electrical balance is provided by concentration gradients which are maintained by a combination of selective permeability characteristics and active pumping mechanism.

The lipid component of the membrane is a double sheet of phospholipids, elongated molecules with polar groups at one end and the fatty acid chains at the other. The ions that carry the currents used for neuronal signaling are among these water-soluble substances, so the lipid bilayer is also an insulator, across which membrane potentials develop. In biophysical terms, the lipid bilayer is not permeable to ions. In electrical terms, it functions as a capacitor, able to store charges of opposite sign that are attracted to each other but unable to cross the membrane. Embedded in the lipid bilayer is a large assortment of proteins. These are proteins that regulate the passage of ions into or out of the cell. Certain membrane-spanning proteins allow selected ions to flow down electrical or concentration gradients or by pumping them across.

These membrane-spanning proteins consist of several subunits surrounding a central aqueous pore (shown in FIG. 10B). Ions whose size and charge “fit” the pore can diffuse through it, allowing these proteins to serve as ion channels. Hence, unlike the lipid bilayer, ion channels have an appreciable permeability (or conductance) to at least some ions. In electrical terms, they function as resistors, allowing a predicable amount of current flow in response to a voltage across them.

A nerve cell can be excited by increasing the electrical charge within the neuron, thus increasing the membrane potential inside the nerve with respect to the surrounding extracellular fluid. As shown in FIG. 11, stimuli 4 and 5 are subthreshold, and do not induce a response. Stimulus 6 exceeds a threshold value and induces an action potential (AP) 17 which will be propagated. The threshold stimulus intensity is defined as that value at which the net inward current (which is largely determined by Sodium ions) is just greater than the net outward current (which is largely carried by Potassium ions), and is typically around −55 mV inside the nerve cell relative to the outside (critical firing threshold). If however, the threshold is not reached, the graded depolarization will not generate an action potential and the signal will not be propagated along the axon. This fundamental feature of the nervous system i.e., its ability to generate and conduct electrical impulses, can take the form of action potentials 17, which are defined as a single electrical impulse passing down an axon. This action potential 17 (nerve impulse or spike) is an “all or nothing” phenomenon, that is to say once the threshold stimulus intensity is reached, an action potential will be generated.

FIG. 12A illustrates a segment of the surface of the membrane of an excitable cell. Metabolic activity maintains ionic gradients across the membrane, resulting in a high concentration of potassium (K⁺) ions inside the cell and a high concentration of sodium (Na⁺) ions in the extracellular environment. The net result of the ionic gradient is a transmembrane potential that is largely dependent on the K⁺ gradient. Typically in nerve cells, the resting membrane potential (RMP) is slightly less than 90 mV, with the outside being positive with respect to inside.

To stimulate an excitable cell, it is only necessary to reduce the transmembrane potential by a critical amount. When the membrane potential is reduced by an amount ΔV, reaching the critical or threshold potential (TP); Which is shown in FIG. 12B. When the threshold potential (TP) is reached, a regenerative process takes place: sodium ions enter the cell, potassium ions exit the cell, and the transmembrane potential falls to zero (depolarizes), reverses slightly, and then recovers or repolarizes to the resting membrane potential (RMP).

For a stimulus to be effective in producing an excitation, it must have an abrupt onset, be intense enough, and last long enough. These facts can be drawn together by considering the delivery of a suddenly rising cathodal constant-current stimulus of duration d to the cell membrane as shown in FIG. 12B.

Cell membranes can be reasonably well represented by a capacitance C, shunted by a resistance R as shown by a simplified electrical model in FIG. 12C, and shown in a more realistic electrical model in FIG. 13, where neuronal process is divided into unit lengths, which is represented in an electrical equivalent circuit. Each unit length of the process is a circuit with its own membrane resistance. (rₘ), membrane capacitance (cₘ), and axonal resistance (rₐ).

When the stimulation pulse is strong enough, an action potential will be generated and propagated. As shown in FIG. 14, the action potential is traveling from right to left. Immediately after the spike of the action potential there is a refractory period when the neuron is either unexcitable (absolute refractory period) or only activated to sub-maximal responses by supra-threshold stimuli (relative refractory period). The absolute refractory period occurs at the time of maximal Sodium channel inactivation while the relative refractory period occurs at a later time when most of the Na⁺channels have returned to their resting state by the voltage activated K⁺current. The refractory period has two important implications for action potential generation and conduction. First, action potentials can be conducted only in one direction, away from the site of its generation, and secondly, they can be generated only up to certain limiting frequencies.

A single electrical impulse passing down an axon is shown schematically in FIG. 15. The top portion of the figure (A) shows conduction over mylinated axon (fiber) and the bottom portion (B) shows conduction over nonmylinated axon (fiber). These electrical signals will travel along the nerve fibers.

The information in the nervous system is coded by frequency of firing rather than the size of the action potential. This is shown schematically in FIG. 16. The bottom portion of the figure shows a train of action potentials 17.

In terms of electrical conduction, myelinated fibers conduct faster, are typically larger, have very low stimulation thresholds, and exhibit a particular strength-duration curve or respond to a specific pulse width versus amplitude for stimulation, compared to unmyelinated fibers. The A and B fibers can be stimulated with relatively narrow pulse widths, from 50 to 200 microseconds (μs), for example. The A fiber conducts slightly faster than the B fiber and has a slightly lower threshold. The C fibers are very small, conduct electrical signals very slowly, and have high stimulation thresholds typically requiring a wider pulse width (300-1,000 μs) and a higher amplitude for activation. Because of their very slow conduction, C fibers would not be highly responsive to rapid stimulation. Selective stimulation of only A and B fibers is readily accomplished. The requirement of a larger and wider pulse to stimulate the C fibers, however, makes selective stimulation of only C fibers, to the exclusion of the A and B fibers, virtually unachievable inasmuch as the large signal will tend to activate the A and B fibers to some extent as well.

As shown in FIG. 17A, when the distal part of a nerve is electrically stimulated, a compound action potential is recorded by an electrode located more proximally. A compound action potential contains several peaks or waves of activity that represent the summated response of multiple fibers having similar conduction velocities. The waves in a compound action potential represent different types of nerve fibers that are classified into corresponding functional categories as shown in the Table one below,

TABLE 1

| | Conduction | Fiber | |
|---|---|---|---|
| Fiber | Velocity | Diameter | |
| Type | (m/sec) | (μm) | Myelination |
| A Fibers | | | |
| Alpha | 70-120 | 12-20 | Yes |
| Beta | 40-70 | 5-12 | Yes |
| Gamma | 10-50 | 3-6 | Yes |
| Delta | 6-30 | 2-5 | Yes |
| B Fibers | 5-15 | <3 | Yes |
| C Fibers | 0.5-2.0 | 0.4-1.2 | No |

FIG. 18B further clarifies the differences in action potential conduction velocities between the Aδ-fibers and the C-fibers. For many of the application of current patent application, it is the slow conduction C-fibers that are stimulated by the pulse generator.

The modulation of nerve in the periphery, as done by the body, in response to different types of pain is illustrated schematically in FIGS. 19 and 20. As shown schematically in FIG. 19, the electrical impulses in response to acute pain sensations are transmitted to brain through peripheral nerve and the spinal cord. The first-order peripheral neurons at the point of injury transmit a signal along A-type nerve fibers to the dorsal horns of the spinal cord. Here the second-order neurons take over, transfer the signal to the other side of the spinal cord, and pass it through the spinothalamic tracts to thalamus of the brain. As shown in FIG. 20, duller and more persistent pain travel by another-slower route using unmyelinated C-fibers. This route made up from a chain of interconnected neurons, which run up the spinal cord to connect with the brainstem, the thalamus and finally the cerebral cortex. The autonomic nervous system also senses pain and transmits signals to the brain using a similar route to that for dull pain.

Vagus nerve stimulation, as performed by the system and method of the current patent application, is a means of directly affecting central function. FIG. 21 shows cranial nerves have both afferent pathway 19 (inward conducting nerve fibers which convey impulses toward the brain) and efferent pathway 21 (outward conducting nerve fibers which convey impulses to an effector). Vagus nerve is composed of approximately 80% afferent sensory fibers carrying information to the brain from the head, neck, thorax, and abdomen. The sensory afferent cell bodies of the vagus reside in the nodose ganglion and relay information to the nucleus tractus solitarius (NTS).

The vagus nerve is composed of somatic and visceral afferents and efferents. Usually, nerve stimulation activates signals in both directions (bi-directionally). It is possible however, through the use of special electrodes and waveforms, to selectively stimulate a nerve in one direction only (unidirectionally), as described later in this disclosure. The vast majority of vagus nerve fibers are C fibers, and a majority are visceral afferents having cell bodies lying in masses or ganglia in the skull.

In considering the anatomy, the vagus nerve spans from the brain stem all the way to the splenic flexure of the colon. Not only is the vagus the parasympathetic nerve to the thoracic and abdominal viscera, it also the largest visceral sensory (afferent) nerve. Sensory fibers outnumber parasympathetic fibers four to one. In the medulla, the vagal fibers are connected to the nucleus of the tractus solitarius (viceral sensory), and three other nuclei. The central projections terminate largely in the nucleus of the solitary tract, which sends fibers to various regions of the brain (e.g., the thalamus, hypothalamus and amygdala).

As shown in FIG. 22, the vagus nerve emerges from the medulla of the brain stem dorsal to the olive as eight to ten rootlets. These rootlets converge into a flat cord that exits the skull through the jugular foramen. Exiting the Jugular foramen, the vagus nerve enlarges into a second swelling, the inferior ganglion.

In the neck, the vagus lies in a groove between the internal jugular vein and the internal carotid artery. It descends vertically within the carotid sheath, giving off branches to the pharynx, larynx, and constrictor muscles. From the root of the neck downward, the vagus nerve takes a different path on each side of the body to reach the cardiac, pulmonary, and esophageal plexus (consisting of both sympathetic and parasympathetic axons). From the esophageal plexus, right and left gastric nerves arise to supply the abdominal viscera as far caudal as the splenic flexure.

In the body, the vagus nerve regulates viscera, swallowing, speech, and taste. It has sensory, motor, and parasympathetic components. Table two below outlines the innervation and function of these components.

TABLE 2
Vagus Nerve Components

| Component fibers | Structures innervated | Functions |
|---|---|---|
| SENSORY | Pharynx. larynx, | General sensation |
| | esophagus, external | |
| | ear | |
| | Aortic bodies, aortic arch | Chemo- and |
| | | baroreception |
| | Thoracic and abdominal | |
| | viscera | |
| MOTOR | Soft palate, pharynx, | Speech, swallowing |
| | larynx, upper esophagus | |
| PARASYMPATHETIC | Thoracic and abdominal | Control of |
| | viscera | cardiovascular |
| | | system, respiratory |
| | | and gastrointestinal |
| | | tracts |

On the Afferent side, visceral sensation is carried in the visceral sensory component of the vagus nerve. As shown in FIGS. 23 and 24, visceral sensory fibers from plexus around the abdominal viscera converge and join with the right and left gastric nerves of the vagus. These nerves pass upward through the esophageal hiatus (opening) of the diaphragm to merge with the plexus of nerves around the esophagus. Sensory fibers from plexus around the heart and lungs also converge with the esophageal plexus and continue up through the thorax in the right and left vagus nerves. As shown in FIG. 15B, the central process of the nerve cell bodies in the inferior vagal ganglion enter the medulla and descend in the tractus solitarius to enter the caudal part of the nucleus of the tractus solitarius. From the nucleus, bilateral connections important in the reflex control of cardiovascular, respiratory, and gastrointestinal functions are made with several areas of the reticular formation and the hypothalamus.

The afferent fibers project primarily to the nucleus of the solitary tract (shown schematically in FIGS. 4 and 2) which extends throughout the length of the medulla oblongata. A small number of fibers pass directly to the spinal trigeminal nucleus and the reticular formation. As shown in FIG. 4, the nucleus of the solitary tract has widespread projections to cerebral cortex, basal forebrain, thalamus, hypothalamus, amygdala, hippocampus, dorsal raphe, and cerebellum. Because of the widespread projections of the Nucleus of the Solitary Tract, neuromodulation of the vagal afferent nerve fibers provide therapy and alleviation of symptoms of depression, and other central nervous system disorders.

### PRIOR ART

U.S. Pat. Nos. 4,702,254, 4,867,164 and 5,025,807 (Zabara) generally disclose animal research and experimentation related to epilepsy and the like. Applicant's method of neuromodulation is significantly different than that disclosed in Zabara '254, '164’ and '807 patents.

U.S. Pat. No. 5,299,569 (Wernicke et al.) is directed to the use of implantable pulse generator technology for treating and controlling neuropsychiatric disorders including schizophrenia, depression, and borderline personality disorder.

U.S. Pat. No. 6,205,359 B1 (Boveja) and U.S. Pat. No. 6,356,788 B2 (Boveja) are directed to adjunct therapy for neurological and neuropsychiatric disorders using an implanted lead-receiver and an external stimulator.

U.S. Pat. No. 5,193,539 (Schulman, et al) is generally directed to an addressable, implantable microstimulator that is of size and shape which is capable of being implanted by expulsion through a hypodermic needle. In the Schulman patent, up to 256 microstimulators may be implanted within a muscle and they can be used to stimulate in any order as each one is addressable, thereby providing therapy for muscle paralysis.

U.S. Pat. No. 5,405,367 (Schulman, et al) is generally directed to the structure and method of manufacture of an implantable microstimulator.

### REFERENCES

1) Salinsky M C, Burchiel K J. Vagus nerve stimulation has no effect on awake EEG rhythms in humans. Epilepsia 1993; 34: 299-304.

2) Hammond E J, Uthman B M, Reid S A, et al. Electrophysiological studies of vagus nerve stimulation in humans, I: EEG effects. Epilepsia 1992; 33 1013-1020.

3) Henry T R, Bakay R A E, Votaw J R, et al. Brain blood flow alterations induced by therapeutic vagus nerve stimulation in partial epilepsy, I acute effects at high and low levels of stimulation. Epilepsia 1998; 39: 983-90.

4) Henry T R, Votaw J R, Pennell P B, et al. Acute blood flow changes and efficacy of vagus nerve stimulation in partial epilepsy. Neurolology 1999: 52: 1166-73.

5) Henry R, Bakay R A E, et al, Brain blood-flow alterations induced by therapeutic vagus nerve stimulation in partial epilepsy: ii) Prolonged effects at high levels of stimulation. Epilepsia vol.45; (9) 2004 pp.1064-1070.

6) Garnett E S, Nahmias C, Scheffel A, et al. Regional cerebral blood flow in man manipulated by direct vagal stimulation. Pacing and Clinical Electrophysiology 1992; 15: 1579-1580.

7) Ko D, Heck C, Grafton S, et al. Vagus nerve stimulation activates central nervous system structures in epileptic patients during PET H₂ ¹⁵O blood flow imaging. Neurosurgery 1966; 39: 426-31.

8) Sackeim H A, Prohovnik I, Mueller J R, Brown R P, Apter S, Prudic J. Devanand D P, Mukherjee S: Regional cerebral blood flow in mood disorder, I: comparison of major depressives and normal controls at rest. Arch Gen Psychiatry 1990; 47: 60-70.

9) Martin S D, Martin E, Rai S S, Richardson M A, Royall R: Brain blood flow changes in depressed patients treated with interpersonal psychotherapy or venlafaxine hydrochloride: preliminary findings. Arch Gen Psychiatry 2001; 58: 641-648.

10) Kalia M, Neurobiological basis of depression: an update. Metabolism clinical and experimental 54 (Suppl. 1) 2005 pp. 24-27

11) Delgado P L, Moreno F A, Role of norepinephrine in depression. J. Clinical Psychiatry 61 (Suppl. 1) 2000 pp. 5-12.

12) Delgado P L, How antidepressants help depression: Mechanisms of action and clinical response J Clinical Psychiatry 2004; 65 (suppl. 4) pp. 25-30.

13) Videbech P, PET measurements of brain glucose metabolism and blood flow in major depressive disorder: a critical review. Acta Psychiatr Scand 2000: 101 pp. 11 -20.

14) Zobel A, Alexius J, et al. Changes in regional cerebral blood flow by therapeutic vagus nerve stimulation in depression: An exploratory approach. Psychiatry Research: Neuroimaging 139 (2005) 165-179.

15) Post R M, DeLisi L E, et al. Glucose utilization in the temporal cortex of affectively ill patients: Positron emmission tomography. Biol. Psychiatry 1987: 22 pp. 545-553.

16) Mayberg H S, Modulating dysfunctional limbic-cortical circuits in depression: towards development of brain-based algorithms for diagnosis and optimised treatment. British Medical Bulletin 2003; 65: 193-207.

17) Groves D A, Brown V J Vagal nerve stimulation: a review of its applications and potential mechanisms that mediate its clinical effects. Neuroscience and Biobehavioral Reviews 29 (2005) 493-500.

18) Drevets W C, Prefrontal cortical-amygdalar metabolism in major depression. Annals New York Academy of Science pp 614-637.

19) Narayanan J T, Watts R, et al. Cerebral activation during vagus nerve stimulation: A functional M R study. Epilepsia, 43(12): 1509-1514, 2002.

### Prior Art Teachings and Applicant's Methodology

The prior art teachings of Zabara and Wernicke in general relies on the fact, that in anesthetized animals stimulation of vagal nerve afferent fibers evokes detectable changes of the EEG in all of the regions, and that the nature and extent of these EEG changes depends on the stimulation parameters. They postulated (Wernicke et al. U.S. Pat. No. 5,269,303) that synchronization of the EEG may be produced when high frequency (>70 Hz) weak stimuli activate only the myelinated (A and B) nerve fibers, and that desynchronization of the EEG occurs when intensity of the stimulus is increased to a level that activates the unmyelinated (C) nerve fibers.

The applicant's methodology is different, and among other things is based on cumulative effects of providing electrical pulses to the vagus nerve(s) its branches or parts thereof. Complex and/or rectangular electrical pulses are provided to vagus nerve(s) to increase and/or decrease rCBF to selective parts/regions of the brain according to the specific nature of the disorder, and/or alter neurochemicals in the brain without regard to synchronization or de-sychronization of patient's EEG. Further, the applicant's invention is based on an open loop system wherein the physician determines the programs and/or parameters for stimulation and/or blocking for the patient.

The means and functionality of the applicant's invention does not rely on VNS-induced EEG changes, and is relevant since an intent of Zabara and Wernicke et al. teachings is to have a feedback system, wherein a sensor in the implantable system responds to EEG changes providing vagus nerve stimulation. Applicant's methodology is based on an open-loop system where the physician determines the parameters/programs for vagus nerve stimulation (and blocking). If the selected parameters or programs are uncomfortable, or are not tolerated by the patient, the electrical parameters are re-programmed. Advantageously, according to this disclosure, some re-programming or parameter adjustment may be done from a remote location, over a wide area network. A method of remote communication for neuromodulation therapy system is disclosed in commonly assigned U.S. Pat. No. 6,662,052 B1 and applicant's co-pending application Ser. No. 10/730,513 (Boveja).

It is of interest that clinical investigation (in conscious humans) have not shown VNS-induced changes in the background EEGs of humans (References 1 and 2, by Salinsky M C and Hammond E J). A study, which used awake and freely moving animals, also showed no VNS-induced changes in background EEG activity. Taken together, the findings from animal study and human studies indicate that acute desynchronization of EEG activity is not a prominent feature of VNS when it is administered during physiologic wakefulness and sleep

One of the advantages of applicant's open-loop methodology is that predetermined/pre-packaged programs may be used. This may be done utilizing an inexpensive implantable pulse generator as disclosed in applicant's commonly owned U.S. Pat. No 6,760,626 B1 referred to as Boveja '626 patent. Predetermined/pre-packaged programs define neuromodulation parameters such as pulse amplitude, pulse width, pulse frequency, on-time and off-time. Examples of predetermined/pre-packaged programs are disclosed in applicant's '626 patent, and in this disclosure for both implantable and external pulse generator means. If an activated pre-determined program is uncomfortable for the patient, a different pre-determined program may be activated or the program may be selectively modified.

Another advantage of applicant's methodology is that, at any given time a patient will receive the most aggressive therapy that is well tolerated. Since the therapy is cumulative the clinical benefits will be realized quicker

Another advantage of applicant's methodology is that complex pulses may be provided. Complex electrical pulses comprises at least one of multi-level pulses, biphasic pulses, non-rectangular pulses, or pulses with varying amplitude during the pulse. Complex pulses may also be used in conjunction with tripolar electrodes. The use of complex pulses adds another dimension to selective stimulation of vagus nerve, as recruitment of different fibers occurs during the pulse. The Zabara and Wernicke teachings utilize rectangular pulses.

In summery, applicant's invention is based on an open-loop pulse generator means utilizing predetermined (pre-packaged programs), where the effects of the therapy and clinical benefits are cumulative effects, which occur over a period of time with selective stimulation. Prior art teachings (of vagal tuning) point away from using predetermined (pre-packaged programs).

In the applicant's methodology, after the patient has recovered from surgery (approximately 2 weeks), and the stimulation/blocking is turned ON, nothing happens immediately. After a few weeks of intermittent stimulation, the effects start to become noticeable in some patients. Thereafter, the beneficial effects of pulsed electrical therapy accumulate up to a certain point, and are sustained over time, as the therapy is continued.

This Application is related to the following co-pending Patent Applications:

| | | Patent/ | Filing date/ |
|---|---|---|---|
| No. | Title | Application | Grant date |
| 1. | Apparatus and method for | 6,356,788 | 03/12/2002 |
| | adjunct (add-on) therapy for | | |
| | depression, migraine, neuro- | | |
| | psychiatric disorders, partial | | |
| | complex epilepsy, generalized | | |
| | epilepsy and involuntary | | |
| | movement disorders utilizing | | |
| | an external stimulator. | | |
| 2. | Apparatus and method for treat- | 6,760,626 | Jul. 6, 2004 |
| | ment of neurological and neuro- | | |
| | psychiatric disorders using | | |
| | programmerless implantable | | |
| | pulse generator system. | | |
| 3. | A method and system for | 10/142,298 | May 9, 2002 |
| | modulating the vagus nerve | | |
| | (10^{th} cranial nerve) using | | |
| | modulated pulses. | | |
| 4. | Method and system for | 10/841995 | 05/08/2004 |
| | modulating the vagus nerve | | |
| | (10^{th} cranial nerve) with | | |
| | electrical pulses using | | |
| | implanted and external | | |
| | components, to provide therapy | | |
| | for neurological and neuro- | | |
| | psychiatric disorders. | | |
| 5. | Method and system for providing | 11/126,673 | May 11, 2005 |
| | adjunct (add-on) therapy for | | |
| | depression, anxiety and | | |
| | obsessive-compulsive disorders | | |
| | by providing electrical pulses | | |
| | to vagus nerve(s). | | |

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram showing the overall structure of the brain.

FIG. 2 is a schematic diagram of the brain showing relationship of the vagus nerve and solitary tract nucleus to other centers of the brain.

FIG. 3 is a schematic diagram depicting connections of vagus nerve with solitary tract nucleus (NTS), parabrachial nucleus, and higher centers in the brain.

FIG. 4 is a simplified block diagram illustrating the connections of solitary tract nucleus to other centers of the brain.

FIGS. 5A and 5B are lateral view of the brain showing structures of the limbic system.

FIG. 6 is a diagram of the brain showing reticular activating system (RAS).

FIG. 7 is a graph showing activity curve on fMRI with periods of vagus nerve stimulation.

FIG. 8 is a diagram of the structure of a nerve.

FIG. 9 is a diagram showing different types of nerve fibers.

FIGS. 10A and 10B are schematic illustrations of the biochemical makeup of nerve cell membrane.

FIG. 11 is a figure demonstrating subthreshold and suprathreshold stimuli.

FIGS. 12A, 12B, 12C are schematic illustrations of the electrical properties of nerve cell membrane.

FIG. 13 is a schematic illustration of electrical circuit model of nerve cell membrane.

FIG. 14 is an illustration of propagation of action potential in nerve cell membrane.

FIG. 15 is an illustration showing propagation of action potential along a myelinated axon and non-myelinated axon.

FIG. 16 is an illustration showing a train of action potentials.

FIG. 17 is a diagram showing recordings of compound action potentials.

FIG. 18 is a schematic diagram showing conduction of first pain and second pain.

FIG. 19 is a schematic illustration showing mild stimulation being carried over the large diameter A-fibers.

FIG. 20 is a schematic illustration showing painful stimulation being carried over small diameter C-fibers

FIG. 21 is a schematic diagram of brain showing afferent and efferent pathways.

FIG. 22 is a schematic diagram showing the vagus nerve at the level of the nucleus of the solitary tract.

FIG. 23 is a schematic diagram showing the thoracic and visceral innervations of the vagal nerves.

FIG. 24 is a schematic diagram of the medullary section of the brain.

FIG; 25 depicts in table form, the peculiarities of different forms of device based therapies for neuropsychiatric disorders

FIG. 26 is a diagram depicting, where a patient receives repetitive Transcranial Magnetic Stimulation (rTMS) to the brain, and pulsed electrical stimulation to vagus nerve(s) with an implanted stimulator.

FIGS. 27A and 27B show placement of ECT electrodes, where a patient receives electroconvulsive therapy (ECT), and pulsed electrical stimulation to vagus nerve(s) with an implanted stimulator.

FIG. 28 is a simplified block diagram depicting supplying amplitude and pulse width modulated electromagnetic pulses to an implanted coil.

FIG. 29 depicts a customized garment for placing an external coil to be in close proximity to an implanted coil.

FIG. 30 is a diagram showing the implanted lead-receiver in contact with the vagus nerve at the distal end.

FIG. 31 is a schematic of the passive circuitry in the implanted lead-receiver.

FIG. 32A is a schematic of an alternative embodiment of the implanted lead-receiver.

FIG. 32B is another alternative embodiment of the implanted lead-receiver.

FIG. 33 shows coupling of the external stimulator and the implanted stimulus-receiver.

FIG. 34 is a top-level block diagram of the external stimulator and proximity sensing mechanism.

FIG. 35 is a diagram showing the proximity sensor circuitry.

FIG. 36A shows the pulse train to be transmitted to the vagus nerve.

FIG. 36B shows the ramp-up and ramp-down characteristic of the pulse train.

FIG. 37 is a schematic diagram of the implantable lead.

FIG. 38A is diagram depicting stimulating electrode-tissue interface.

FIG. 38B is diagram depicting an electrical model of the electrode-tissue interface.

FIG. 39 is a schematic diagram showing the implantable lead and one form of stimulus-receiver.

FIG. 40 is a schematic block diagram showing a system for neuromodulation of the vagus nerve, with an implanted component which is both RF coupled and contains a capacitor power source.

FIG. 41 is a simplified block diagram showing control of the implantable neurostimulator with a magnet.

FIG. 42 is a schematic diagram showing implementation of a multi-state converter.

FIG. 43 is a schematic diagram depicting digital circuitry for state machine.

FIGS. 44A-C depicts various forms of implantable microstimulators.

FIG. 45 is a figure depicting an implanted microstimulator for providing pulses to vagus nerve.

FIG. 46 is a diagram depicting the components and assembly of a microstimulator.

FIG. 47 shows functional block diagram of the circuitry for a microstimulator.

FIG. 48 is a simplified block diagram of the implantable pulse generator.

FIG. 49 is a functional block diagram of a microprocessor-based implantable pulse generator.

FIG. 50 shows details of implanted pulse generator.

FIGS. 51A and 51B shows details of digital components of the implantable circuitry.

FIG. 52A shows a schematic diagram of the register file, timers and ROM/RAM.

FIG. 52B shows datapath and control of custom-designed microprocessor based pulse generator.

FIG. 53 is a block diagram for generation of a pre-determined stimulation pulse.

FIG. 54 is a simplified schematic for delivering stimulation pulses.

FIG. 55 is a circuit diagram of a voltage doubler.

FIG. 56A is a diagram depicting ramping-up of a pulse train.

FIG. 56B depicts rectangular pulses.

FIGS. 56C, 56D, and 56E depict multi-step pulses.

FIGS. 56F, 56G, and 56H depict complex pulse trains.

FIG. 56-I depicts the use of tripolar electrodes.

FIGS. 56J and 56K depict step pulses used in conjunction with tripolar electrodes.

FIGS. 56L and 56M depict biphasic pulses used in conjunction with tripolar pulses.

FIGS. 56N and 56-O depict modified square pulses to be used in conjunction with tripolar electrodes.

FIG. 57A depicts an implantable system with tripolar lead for selective unidirectional blocking of vagus nerve stimulation

FIG. 57B depicts selective efferent blocking in the large diameter A and B fibers.

FIG. 57C is a schematic diagram of the implantable lead with three electrodes.

FIG. 57D is a diagram depicting electrical stimulation with conduction in the afferent direction and blocking in the efferent direction.

FIG. 57E is a diagram depicting electrical stimulation with conduction in the afferent direction and selective organ blocking in the efferent direction.

FIG. 57F is a diagram depicting electrical stimulation with conduction in the efferent direction and selective organ blocking in the afferent direction.

FIG. 58 depicts unilateral stimulation of vagus nerve at near the diaphram level.

FIGS. 59A and 59B are diagrams showing communication of programmer with the implanted stimulator.

FIGS. 60A and 60B show diagrammatically encoding and decoding of programming pulses.

FIG. 61 is a simplified overall block diagram of implanted pulse generator (IPG) programmer.

FIG. 62 shows a programmer head positioning circuit.

FIG. 63 depicts typical encoding and modulation of programming messages.

FIG. 64 shows decoding one bit of the signal from FIG. 63.

FIG. 65 shows a diagram of receiving and decoding circuitry for programming data.

FIG. 66 shows a diagram of receiving and decoding circuitry for telemetry data.

FIG. 67 is a block diagram of a battery status test circuit.

FIG. 68 is a diagram showing the two modules of the implanted pulse generator (IPG).

FIG. 69A depicts coil around the titanium case with two feedthroughs for a bipolar configuration.

FIG. 69B depicts coil around the titanium case with one feedthrough for a unipolar configuration.

FIG. 69C depicts two feedthroughs for the external coil which are common with the feedthroughs for the lead terminal.

FIG. 69D depicts one feedthrough for the external coil which is common to the feedthrough for the lead terminal.

FIG. 70 shows a block diagram of an implantable stimulator which can be used as a stimulus-receiver or an implanted pulse generator with rechargeable battery.

FIG. 71 is a block diagram highlighting battery charging circuit of the implantable stimulator of FIG. 70.

FIG. 72 is a schematic diagram highlighting stimulus-receiver portion of implanted stimulator of one embodiment.

FIG. 73A depicts bipolar version of stimulus-receiver module.

FIG. 73B depicts unipolar version of stimulus-receiver module.

FIG. 74 depicts power source select circuit.

FIG. 75A shows energy density of different types of batteries.

FIG. 75B shows discharge curves for different types of batteries.

FIG. 76 depicts externalizing recharge and telemetry coil from the titanium case.

FIGS. 77A and 77B depict recharge coil on the titanium case with a magnetic shield in-between.

FIG. 78 shows in block diagram form an implantable rechargable pulse generator.

FIG. 79 depicts in block diagram form the implanted and external components of an implanted rechargable system.

FIG. 80 depicts the alignment function of rechargable implantable pulse generator.

FIG. 81 is a block diagram of the external recharger.

FIG. 82 depicts remote monitoring of stimulation devices.

FIG. 83 is an overall schematic diagram of the external stimulator, showing wireless communication.

FIG. 84 is a schematic diagram showing application of Wireless Application Protocol (WAP).

FIG. 85 is a simplified block diagram of the networking interface board.

FIGS. 86A and 86B are simplified diagrams showing communication of modified PDA/phone with an external stimulator via a cellular tower/base station.

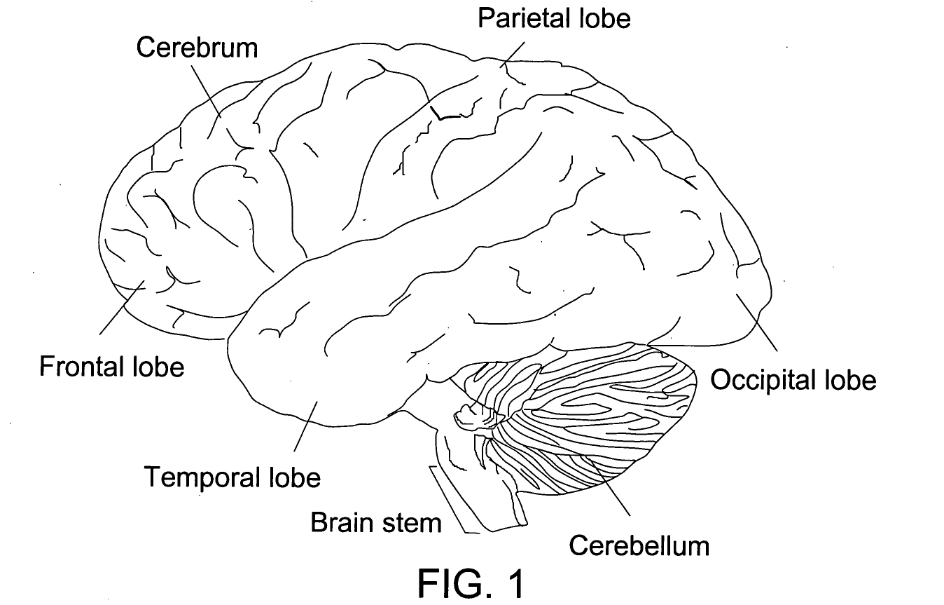

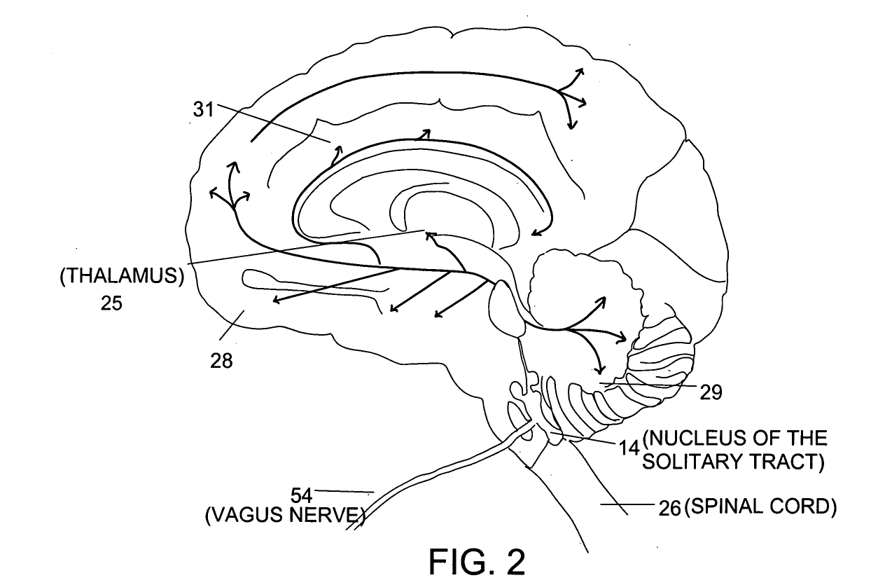

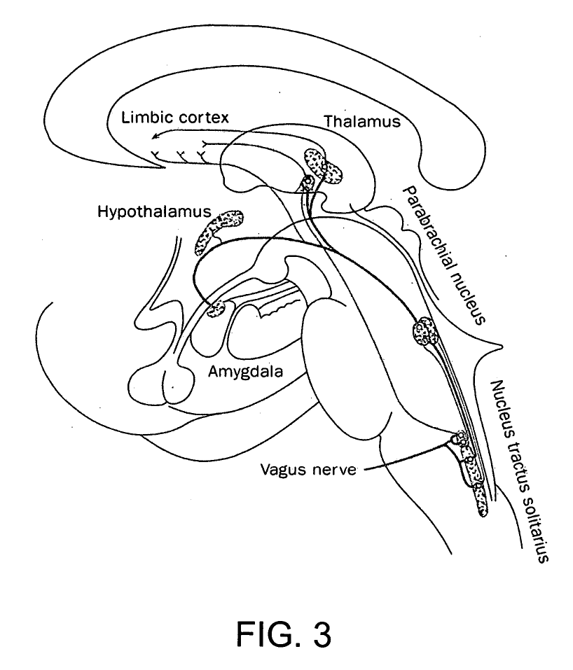

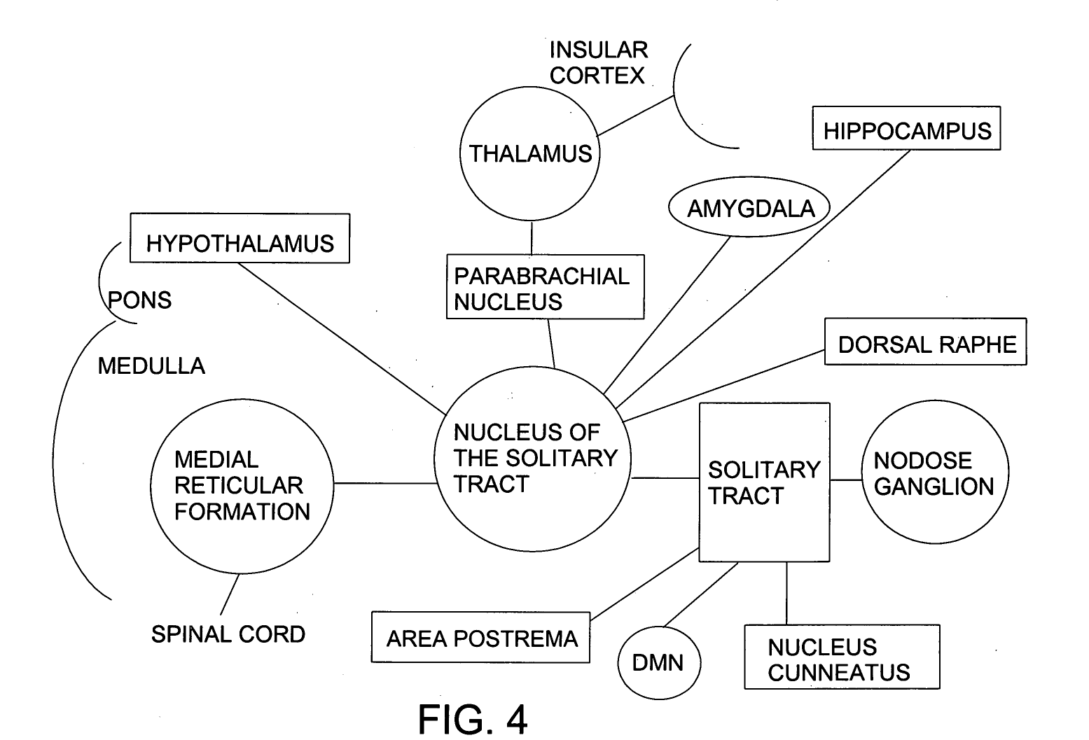

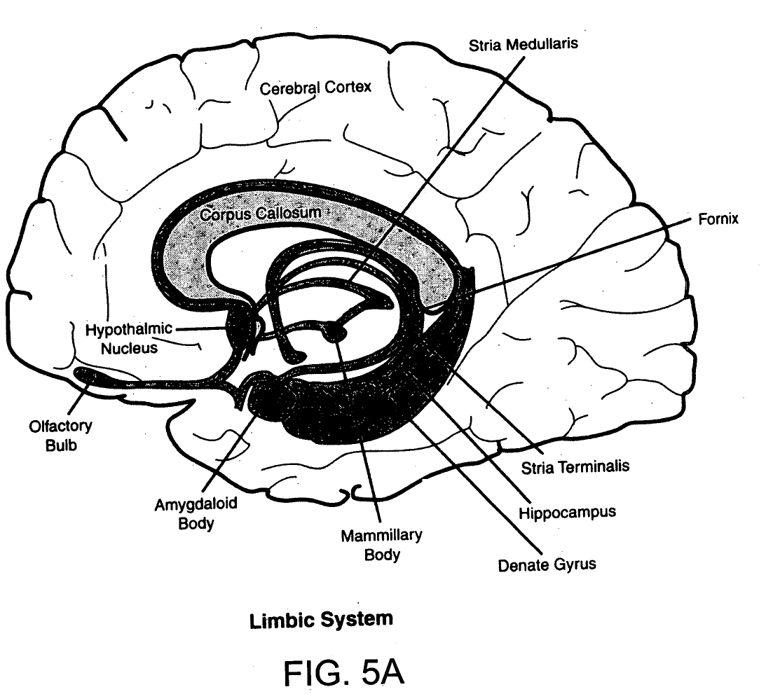

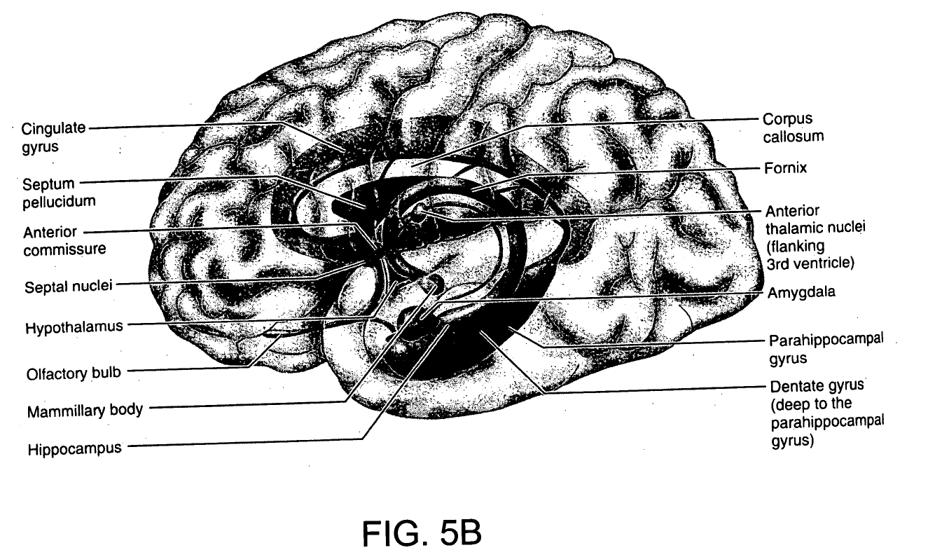

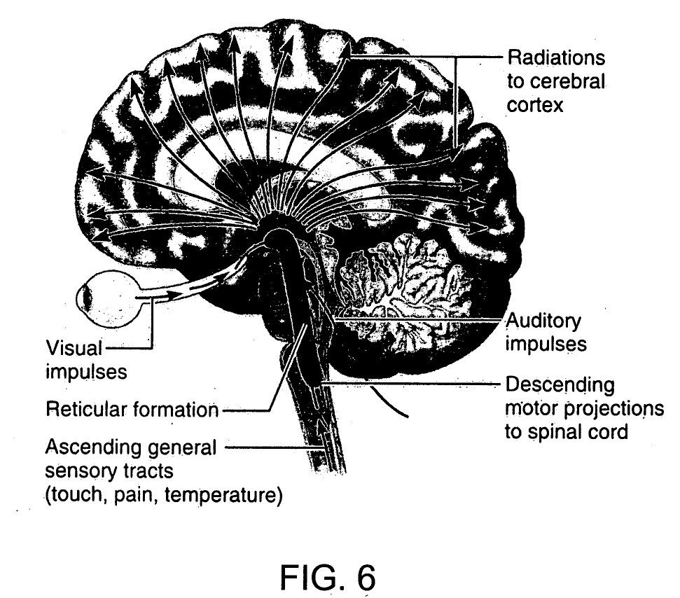

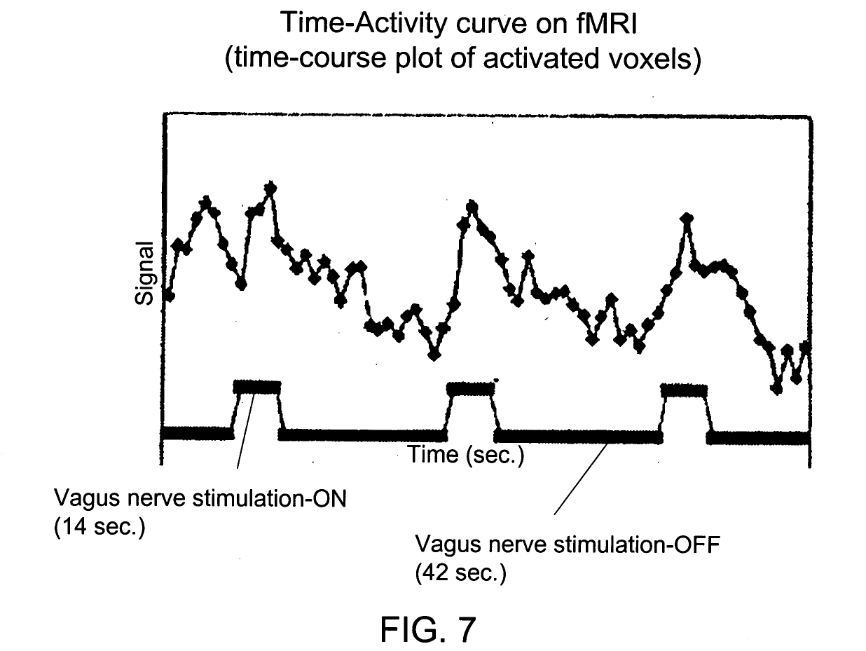

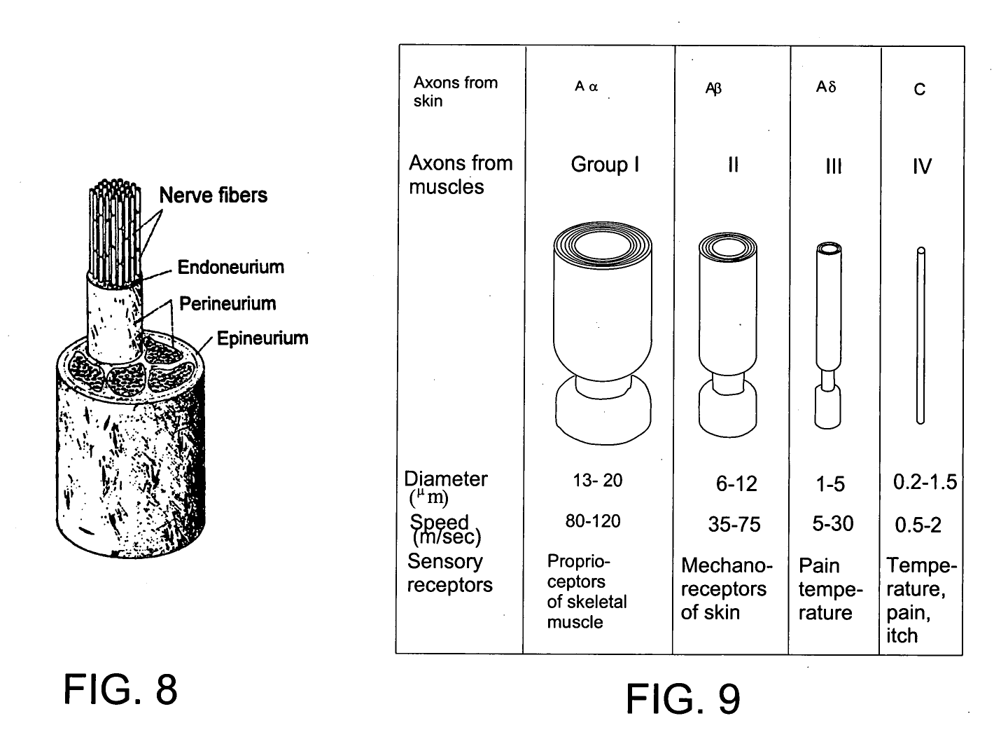

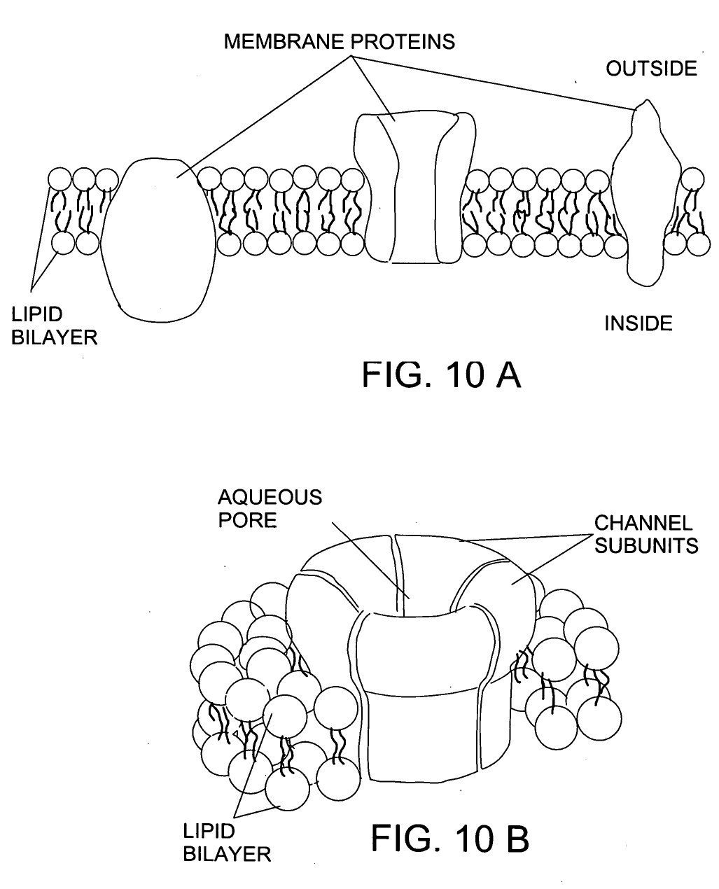

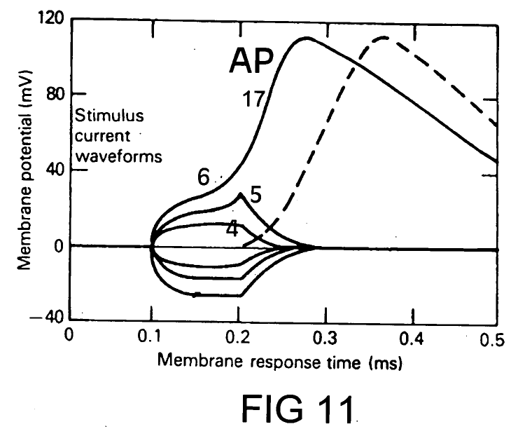

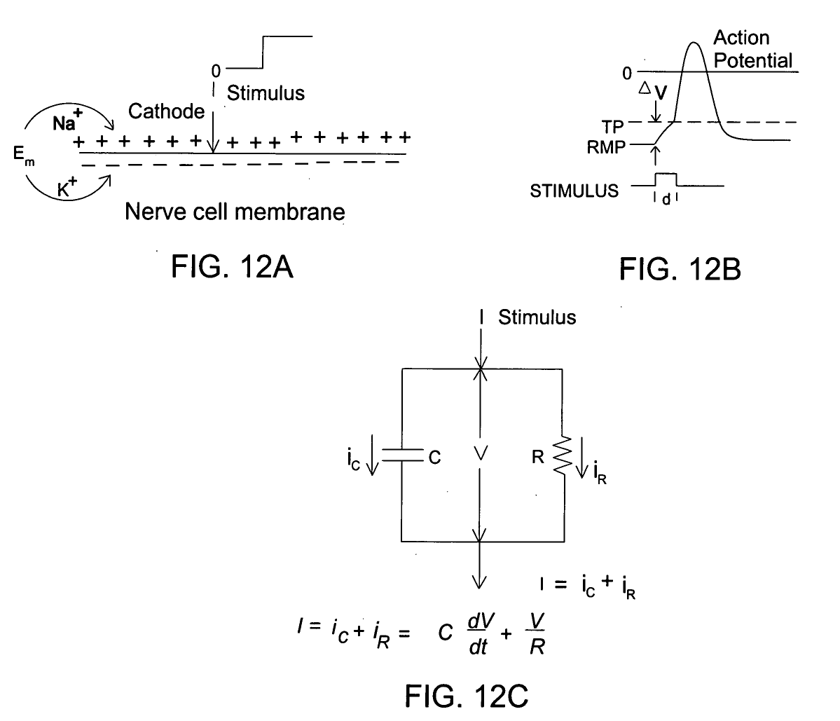

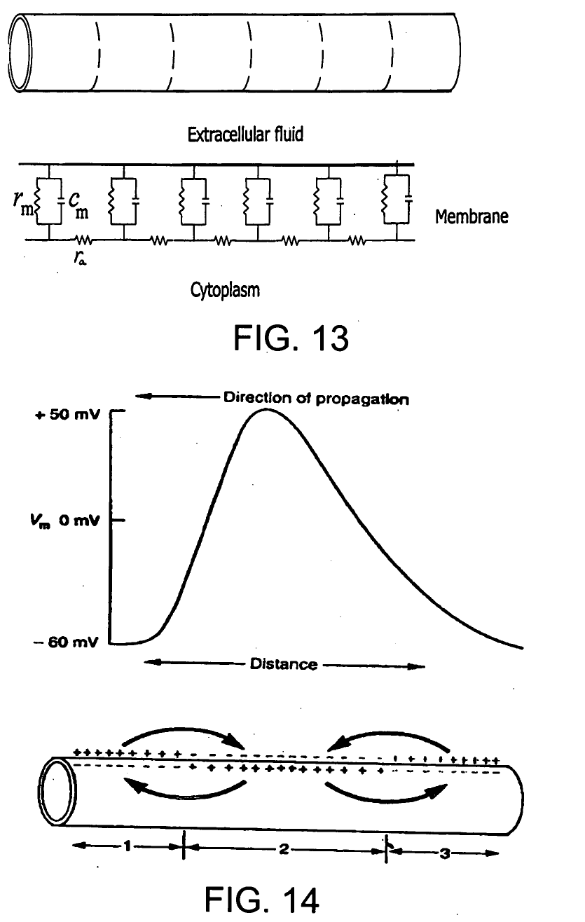

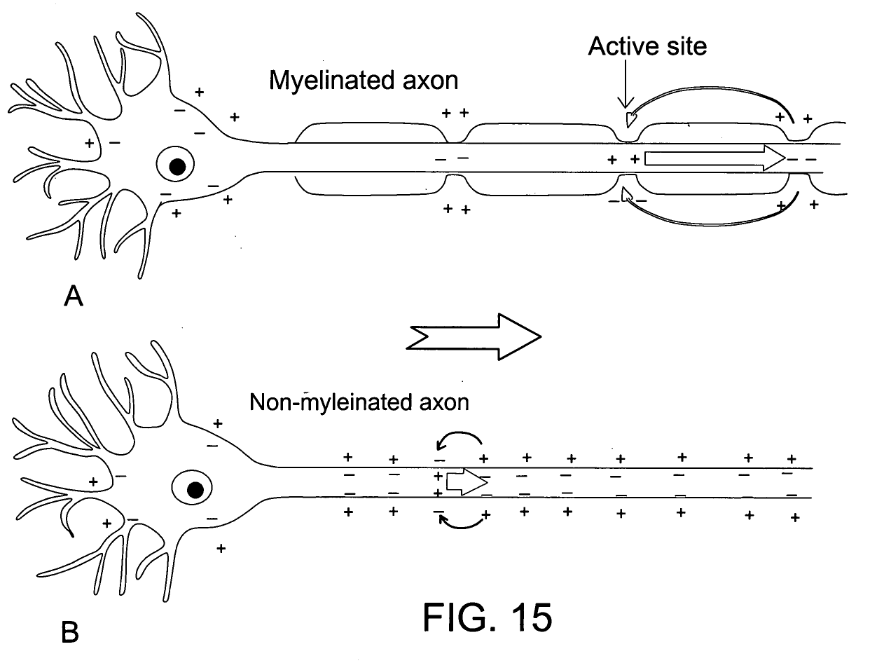

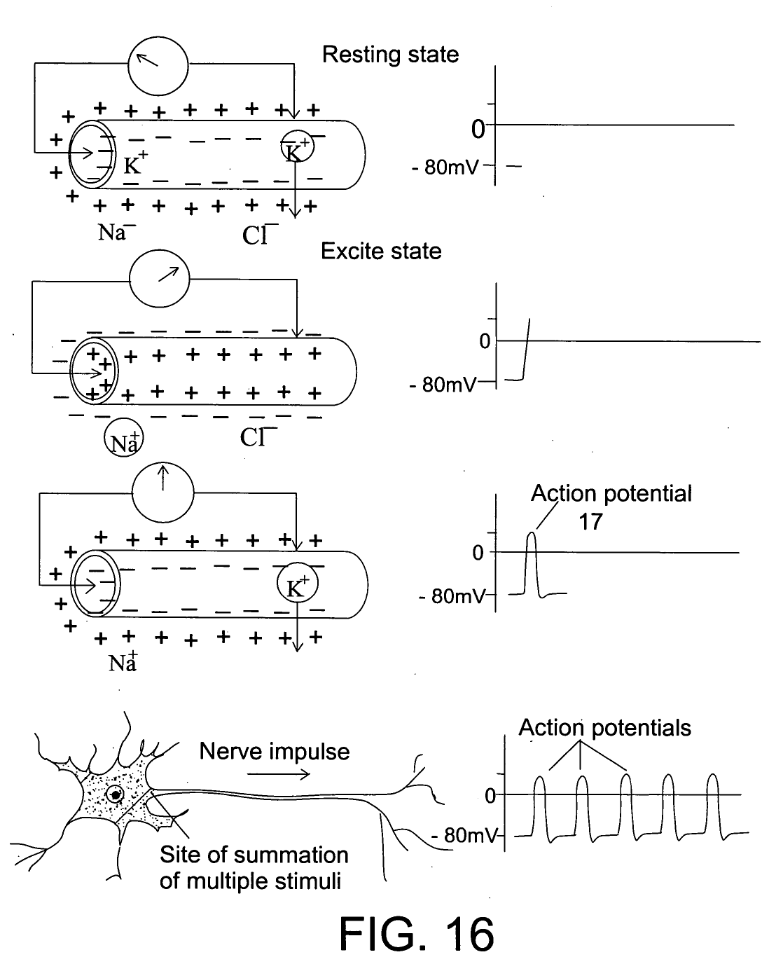

### DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

### Table of Contents

a) Clinical effects of afferent VNS on regional cerebral blood flow and on neurochemicals.

b) Afferent VNS used with transcranial magnetic stimulation (TMS).

c) ECT used with afferent vagus nerve stimulation for depression.

d) Pulse generator means:
i) an implanted stimulus-receiver with an external stimulator;
ii) an implanted stimulus-receiver comprising a high value capacitor for storing charge, used in conjunction with an external stimulator;
iii) a programmer-less implantable pulse generator (I PG) which is operable with a magnet;
iv) a microstimulator;
v) a programmable implantable pulse generator;
vi) a combination implantable device comprising both a stimulus-receiver and a programmable IPG; and
vii) an IPG comprising a rechargeable battery.

e) Remote communications module.

In the method and system of this application, selective pulsed electrical stimulation is applied to vagus nerve(s) for afferent neuromodulation to provide therapy for depression, and other central nervous system (CNS) disorders. An implantable lead is surgically implanted in the patient. The vagus nerve(s) is surgically exposed and isolated. The electrodes on the distal end of the lead are wrapped around the vagus nerve(s), and the terminal (proximal) end of the lead is tunneled subcutaneously. A pulse generator means is connected to the terminal (proximal) end of the lead, and implanted in a subcutaneous pocket. The power source may be external, implantable, or a combination device. Clinical effects of afferent VNS on regional cerebral blood flow (rCBF) and on neurochemicals

Traditionally, depressions have been divided into primary or functional disorders and secondary or organic diseases, but this distinction has gradually become blurred with the advances in neuroimaging techniques. Functional neuroimaging of depressed patients has been used to investigate pathophysiological mechanisms and the physiological basis of the clinical response to antidepressive treatment. The pathophysiology of depression has been extensively investigated by neuroimaging techniques.

Major depressive disorder is clinically, etiologically, and most probably also pathophysiologically heterogeneous. Several neurotransmitters are presumably involved and it is possible that specific syndromes or symptoms of depression are related to unique neurotransmitter deficits. Subgrouping of depressed patients by means of neuroimaging may also help differentiate between patient populations with different treatment needs and different prognoses.

The main finding of the reviewed studies is that patients with major depression have reduced blood flow and glucose metabolism in the prefrontal cortex, anterior cingulate cortex and caudate nucleus when scanned in the resting state and during stressful tests. Apparently, most of these abnormalities are normalized when the patient is cured from the depression. A few abnormalities, however persist representing trait markers. The prefrontal blood flow is negatively correlated with psychomotor retardation. This deficit may be analogous to the symptoms seen in patients with focal lesion of the frontal lobes, who develop apathy and difficulties of planning and initiating behavior, and the findings suggest a pathophysiological mechanism behind the abnormalities in attention often described in patients with major depression. It remains unsettled whether unipolar and bipolar depressions can be distinguished on the basis of functional neuroimaging studies. The literature has, however, significant weaknesses of subject selection, selection of the control group, imaging protocol and image analysis tools employed. No study was designed to control for the possible confounding effects introduced by brain anatomical abnormalities, such as white matter lesions. Few combined the PET with MRI scans, to achieve optimal co-registration of the PET images and to control for systematic structural differences among and between patients and controls.

Positron emission tomography (PET), single-photon emission computed tomography (SPECT), and functional magnetic resonance imaging (fMRI) are three different kinds of functional imaging studies that are dependent on cerebral blood flow. fMRI has advantages, as a technique, compared with PET and SPECT because fMRI avoids the use of radiopharmaceuticals, is noninvasive, and easier to perform.

Differences of regional cerebral blood flow (rCBF) at rest as assessed by positron emission tomography (PET) or single photon emission-computed tomography (SPECT) between patients and controls were reported in a variety of defined brain areas that might be involved in the pathogenesis of depression, e.g., brain structures implicated in mediating emotional and stress responses such as the amygdala, posterior orbital cortex and anterior cingulate cortex as well as areas implicated in attention and sensory processing, such as the dorsal anterior cingulum. In general, a reciprocal limbic-cortical relationship with limbic increase of blood flow is reported in depressed patients compared with controls.

It has been shown that abnormal blood flow patterns were normalized during successful antidepressant treatment as demonstrated by multiple previous reports (published by Drevets, in 2000 in the Annals of the New York Academy of Sciences 877, pp. 614-637; and published by Mayberg, in 2003 in the British Medical Bulletin vol. 65, pp. 193-207). Most areas considered to be involved in depression reveal treatment-induced blood flow changes. Yet, there is variability across specific treatments, e.g., between pharmacological treatment modalities and brain-stimulation methods.

Most reports propose that successful pharmacotherapy induces a reduction of rCBF in limbic regions, while increased blood flow in the dorsolateral prefrontal cortex.

The fibers of the vagus nerve project to limbic and neocortical structures through serotonergic and noradrenergic nuclei of the brain stem, particularly through the nucleus of the tractus solitarius (NTS). The NTS projects to limbic structures such as the subgenual cingulate cortex, which has extensive reciprocal connections with the orbital cortes (OFC) as well as with the hypothalamus, amygdala, nucleus accumbens, ventral trigmenal area, substrantia nigra, nuclei raphe, locus coeruleus and periaqueductal gray matter. Thus, VNS has the potential to modify neuronal activity and rCBF in cortical and limbic structures that are considered to be relevant to depression.

VNS-induced blood flow changes were initially explored in patients with epilepsy. Independent of measurement modalities, the most consistent increase of blood flow was revealed in frontal, temporal and insular cortices, and a decrease was observed in the limbic regions such as hippocampus, amygdala and POC. These observations were published by Henry et al. in 1998, Vonck et al. in 2000, Bohning et al. in 2001, and Van Laere et al. in 2002.

Although vagus nerve stimulation has a very different mechanism of action, it reveals similarities in changes of rCBF to those associated with pharmacological treatment, that is:

1) The region with rCBF increase was the middle frontal gyrus; this region can also be ascertained in responders in some, but not all pharmacological studies; and

2) Reduction of rCBF is observed in the limbic system and associated regions, particularly hippocampus, amygdala, subgenual and ventral anterior cingulum, posterior orbitofrontal cortex and anterior inferior temporal lobes very similar to pharmacological studies (published by Kocmur et al., 1998; Brody et al, 1999, 2001; Drevets, 2000, 2001; Mayberg et al., 2000; Kennedy et al., 2001; Davies et al., 2003; Mayberg, 2003); the decreases in these areas were reported to be more prominent on the left side.

Finally, most striking was the absence of major similarities with other, albeit more widespread, brain-stimulation techniques with antidepressant effects (mainly ECT), indicating a relatively specific antidepressant mode of action of VNS.

In 1999, Henry et al. published an article in the journal Neurology (volume 52, pp. 1166-1173) which showed that VNS acutely induces rCBF alteration at sites that receive vagal afferents and higher-order projections. Most vagal afferents synapse in the nucleus of the tractus solitarius (NTS), and both vagal fibers and axons originating in the NTS project densely to the medullary reticular formation, which has polysyaptic ascending projection to the nucleus reticularis thalami (NRT). The NRT projects to most of the thalamic nuclei, and can synchronize efferent activities of thalamocortical relay neurons in different thalamic nuclei. Thus, ascending influences on the GABAergic neurons of the NRT, perhaps including activities that are altered by VNS, can affect the entire cortex via the thalamocortical relay neurons. The NTS also projects densely to the parabrachial nucleus of the pons, which projects heavily to thalamic intralaminar nuclei, which themselves project diffusely over cerebral cortex.

It was shown that VNS acutely induces rCBF alteration at sites that receive vagal afferents and higher-order projections, including dorsal medulla, somatosensory cortex (contralateral to stimulation), thalamus and cerebellum bilaterally, and several limbic structures (including hippocampus and amygdala bilaterally).

Electrical stimulation of the peripheral vagus nerve requires synaptic transmission to mediate therapeutic activity. Regional alternations in synaptic activity cause rapid changes in regional cerebral blood flow (rCBF). Changes in CBF can be measured over seconds or minutes with functional imaging techniques, including PET, in humans. Rapidly reversible changes in rCBF primarily reflect changes in transsynaptic neurotransmission, in the absence of state changes, seizures, acute ischemia, and other brain vascular dysfunctions. Activation PET techniques showed that left cervical VNS acutely increases synaptic activity in the area of the vagal complex of the dorsal medulla, bilaterally in the thalami and other structures that receive direct projections from the medullary vagal complex, and unilaterally in areas that process left-sided somatosensory information, in human partial epilepsy. These studies also showed that VNS acutely alters synaptic activity in multiple limbic system structures bilaterally, with bilateral CBF increases in the insular and inferior frontal cortices, and bilateral CBF decreases in the hippocampi, amygdala, and posterior cingulate gyri. Patients in the group that received a higher energy of vagus electrical stimulation had greater volumes of activation and deactivation sites than did those in the group that received a lower energy of stimulation. Studies of chronic VNS effects on rCBF showed much smaller volumes of significant rCBF alteration than were found on PET studies of acute VNS. The patient groups and several technical aspects of PET studies differed between the acute VNS-activation and chronic VNS-activation PET studies. Possibly, the differences in rCBF activation between acute and chronic conditions are due in part to chronic adaptation of central processing to VNS, which may tend to attenuate higher cortical and subcortical responses to individual trains of VNS.

Changes in rCBF during trains of VNS, measured early during VNS therapy probably reflect acute VNS-induced changes in regional synaptic activity, and therefore reflect activity in central pathways that have not been modified by long-term adaptations of central processes to chronic VNS.

The imaging data shows that abnormalities in regional cerebral blood flow (rCBF) accompany depression and are altered by treatment. In a study published by Sackeim et al. in Arch Gen Psychiatry (vol. 47, January 1990, Sackeim et al.) on regional cerebral blood flow in mood disorders, it was found that patients with major depressive disorder had both a global flow deficit and an abnormal regional distribution. Further, the reduction in global flow was marked, with the depressed sample averaging a 12% lower rate compared with controls. The average global reduction in depressed patients was of the same order of magnitude as that seen in some of cerebrovascular disease and Alzheimer's disease.

Garnett et al. published a study in the journal PACE in 1992, which also studied regional cerebral blood flow in five patients in whom a vagal stimulator had been implanted on the left hand side. They found significant changes in rCBF (p<0.001) recorded in the region of the anterior thalamus and in the cingulate gyrus anteriorly. The changes in thalamic and cortical blood flow were both on the same side as the vagal stimulation and were encompassed by areas of less significant. (P<0.07) change.

In a study published by Narayanan et al. in 2002 in Epilepsia (vol. 43 pp. 1509-1514), on cerebral activation during vagus nerve stimulation (VNS), they found that patients with VNS had decreased flow to the left-sided (ipsilateral) thalamus. With PET, patients treated with VNS showed acute and chronic changes in cortical and subcortical cerebral blood flow bilaterally. Specifically, there were bilateral increases in cerebral blood flow in the thalamus and hypothalamus and decreases, bilaterally, in the hippocampus and amygdala. Acute VNS-induced cerebral blood flow changes decline over most cortical regions but persist over most subcortical regions.

In one study, fMRI was studied in five patients with VNS stimulation. All five patients showed robust short-term VNS-induced activation in bilateral thalami, ipsilateral more than contralateral, as well as bilateral insular cortices. Activation also was seen in ipsilateral basal ganglia and postcentral gyrus, contralateral superior temporal gyrus, and inferomedial occipital gyri, ipsilateral more than contralateral.

PET studies, which have a spatial resolution of approx. 8 mm and a temporal resolution of summed activity over 1-20 min, have shown VNS-induced cerebral blood flow (CBF) changes. Short-term effects of VNS on regional CBF was studied in 10 patients by Henry et al. These patients had a PET scan before the VNS was implanted, and then within 20 h of VNS activation. There were two main groups of patients in this study, one set with high levels of stimulation and one with low levels. Both sets of patients showed significant blood-flow increases in the dorsocentral medulla, right thalamus, right postcentral gyrus, bilateral insular cortices, hypothalami, and bilateral inferior cerebellar regions. In general, the higher-stimulation group had larger volumes of activation over both cerebral hemispheres than did the low-stimulation group. The high-stimulation group also showed significant blood-flow increases in bilateral orbitofrontal gyri, right entorhinal cortex, and right temporal pole, which were not seen in the low-stimulation group. Both groups of patients had significant decreases in blood flow in bilateral amygdala, hippocampi, and posterior cingulate gyri.

These VNS-related PET activation data were further analyzed by comparing changes in seizure frequencies during 3 months of ongoing VNS with short-term VNS-induced regional CBF changes. They found that only the right and left thalami showed significant association of CBF change with change in seizure frequency.

Three recent PET studies have examined the long-term effects of VNS on regional CBF. Patient-selection criteria and imaging techniques are different in each study. Garnett et al. had reported that VNS activated left thalamus and left anterior cingulate gyri in five patients. In this study, two of the five patients had seizures during data acquisition, which may have influenced the measurements. Ko et al. had reported that VNS activated blood flow in the right thalamus, right posterior temporal cortex, left putamen, and left inferior cerebellum in three patients. Henry et al. restudied their patients after 3 months of ongoing VNS. They found that prolonged VNS-activation PET detected increases in CBF in many of the same regions that had shown increases in the short term, including bilateral thalami, hypothalmi, dorsal-rostral medulla in the high-stimulation group, bilateral inferior cerebellum, bilateral inferior parietal lobules and right postcentral gyrus. In general, they found that subcortical regions, which showed the CBF changes in the short-term study, persisted in showing the same activation in the long-term VNS study, but the cortical changes in CBF did not persist.

Functional MRI with its spatial resolution of ≦2 mm and temporal resolution for single acquisition of ≦1 ms is very suitable for VNS-induced activation studies. In one study by Bohning et al., fMRI was used to study effects of VNS on regional CBF in nine patients with depression who had VNS implanted for a duration of 2 weeks to 23 months. Their VNS settings were diverse, and they were taking a variety of antidepressant medications. This study found BOLD response to VNS in bilateral orbitofrontal and parieto-occipital cortices, left temporal cortex, amygdala, and the hypothalamus.

Neurochemicals

In the mid-1980's it was discovered that selective serotonin reuptake inhibitors (SSRIs) were effective antidepressants. Much research has also focused on trying to understand the role of serotonin (5-HT) in the etiology of depression and its mechanism of antidepressant action. It is known that the enhancement of noradrenergic or serotonergic neurotransmission improves the symptoms of depression.

VNS has been shown to result in a long-lasting (greater than 80-min) increase in release of noradrenaline in the basolateral amygdala, the origin of which could be the locus coeruleus, the largest population of noradrenergic neurons in the brain and in receipt of projections from the nucleus of the solitary tract (Van Bockstaele et al., 1999), thus could be modulated by the vagus. Alternatively, it is also possible that noradrenaline in the amygdala is increased by the direct projections of the noradrenergic neurons of the nucleus of the solitary tract (the A2 noradrenergic cell group), which project to the amygdala (Herbert and Saper, 1992) as well as the locus coeruleus.

### Afferent Vagus Nerve Stimulation (VNS) Used with Transcranial Magnetic Stimulation (TMS)

In one aspect of the invention, afferent vagus nerve stimulation may be used with other pharmacological and non-pharmacological therapies. Drug therapy is typically the first line treatment for depression. Non-pharmacological treatments such as ECT and/or transcranial magnetic stimulation are particularly useful with afferent vagus nerve stimulation. Since ECT and transcranial magnetic stimulation approach the electrical or magnetic stimulation from outside the brain and vagus nerve stimulation approaches the brain from the inside. TMS and ECT also work via different mechanism than vagus nerve stimulation. Applicant's co-pending application Ser. No. 11/074,130 entitled “Method and system for providing therapy for neuropsychiatric and neurological disorder utilizing transcranial magnetic stimulation and pulsed electrical vagus nerve(s) stimulation”, is incorporated herein by reference.

FIG. 25 (shown in table form) generally highlights some of the advantages and disadvantages of various forms of non-pharmacological interventions for the treatment of depression. Considering the advantages and disadvantages of different existing treatments, as shown in conjunction with FIG. 25, a combination of rTMS therapy which involves changing magnetic fields and pulsed electrical vagus nerve stimulation is an ideal combination for device based interventions. The initiation and delivery of these two interventions may be in any sequence or combination, and may be in addition to any drug therapy, as determined by the physician. For example, a patient implanted with vagal nerve stimulator may be given rTMS therapy, or alternatively a patient receiving rTMS therapy may be implanted with a vagus nerve stimulator. Of course, this may be in addition to any drug therapy that may be given to a patient.

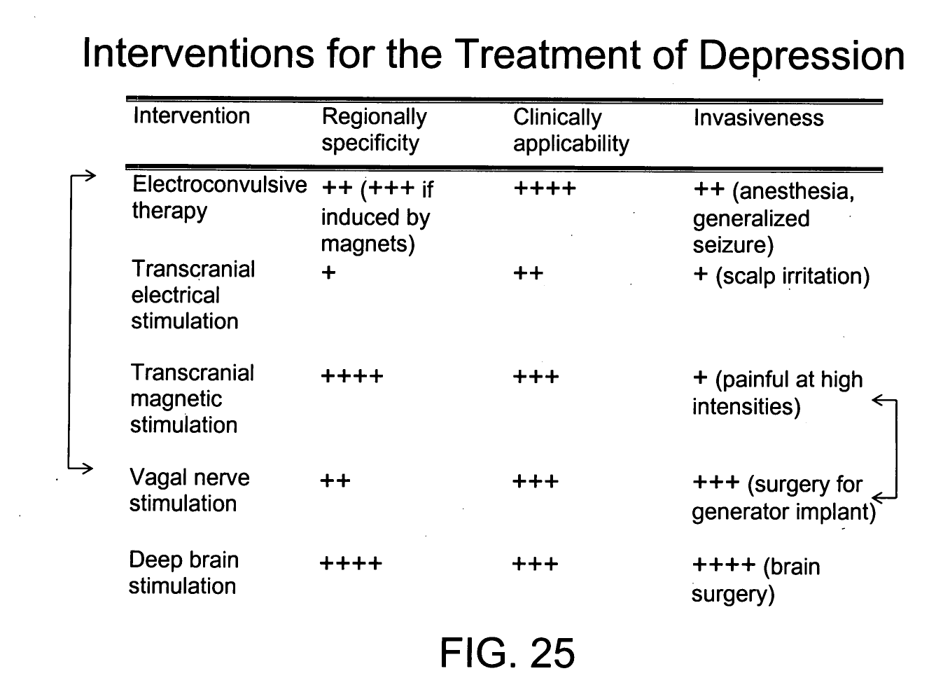

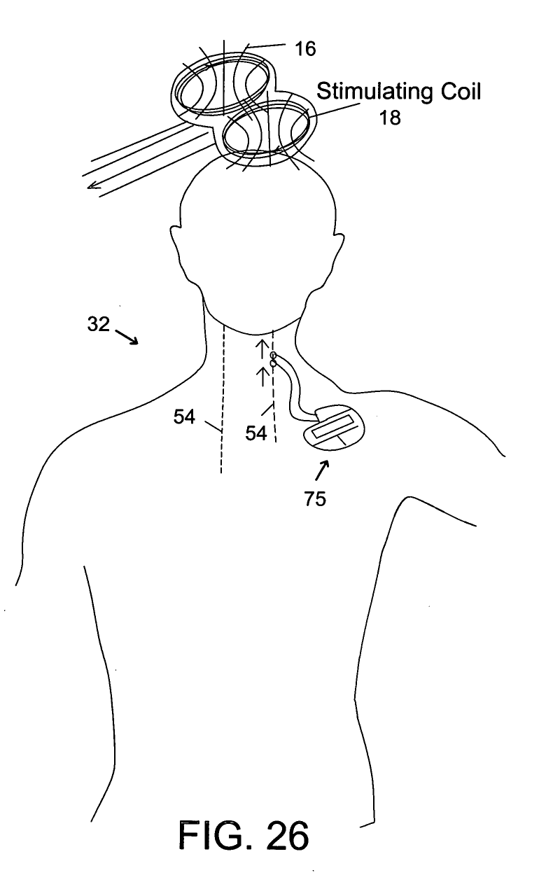

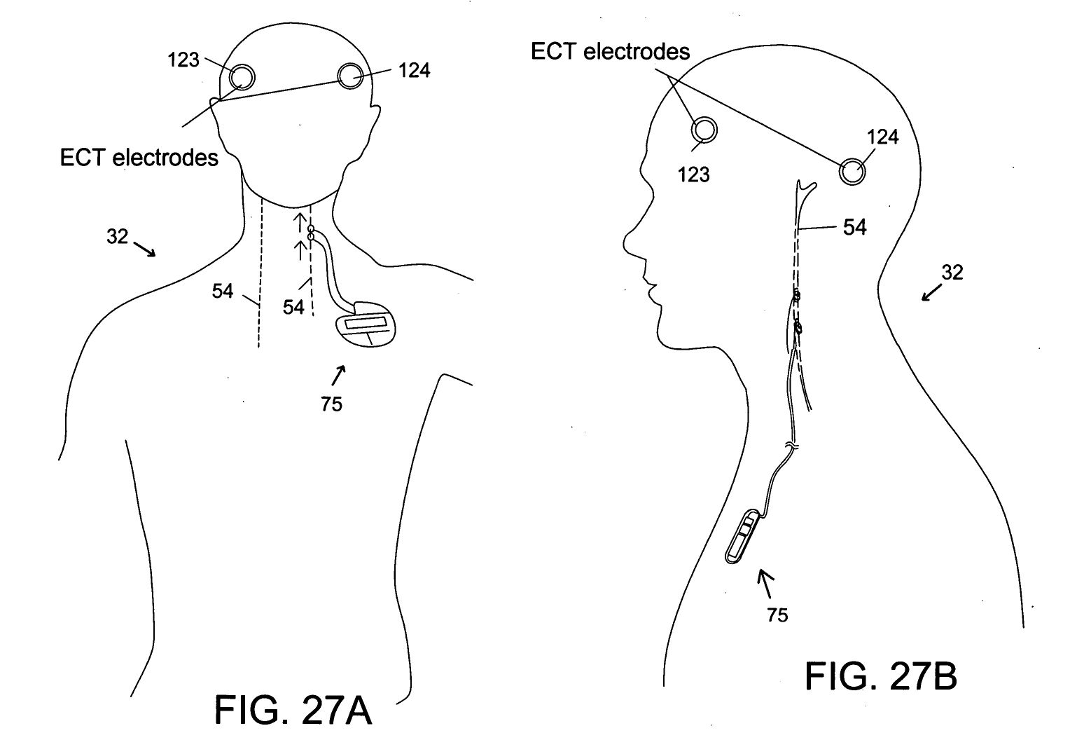

The combination use of rTMS and VNS is depicted in conjunction with FIG. 26. In the method of this application, the beneficial effects of rTMS and VNS would be synergistic or at least additive. The rationale for the combined systems is that with rTMS the electromagnetic energy is penetrated from outside to inside in changing magnetic fields, and with VNS the electrical pulses are delivered to the vagus nerve(s) 54, which provides stimulation (neuromodulation) from inside (i.e. from vagus nerve to brain stem to other projections in the brain). Further, the efficacy and invasiveness of the two stimulation therapies are also matched to provide the patient with balanced risk/benefit ratio. Electrical pulses to the vagus nerve(s) 54 are supplied using a pulse generator means and a lead with electrodes in contact with nerve tissue. rTMS are typically applied in short sessions. Vagus nerve stimulation is typically applied 24 hours/day, 7 days a week, in repeating cycles. The time periods of either rTMS or VNS may vary by any amount at the discretion of the physician.

Also shown in conjunction with Table-3, this combination balances the invasiveness, regional specificity and clinical applicability, and may be with or without concomitant drug therapy. rTMS typically provides immediate benefits of mood improvement and no known side effects, but the benefits may or may not be very long lasting. With VNS the time profile of anti-depressant benefits are sustained over a long period of time, even though they may be slow to accumulate. Therefore, advantageously the combined benefits are both immediate and long lasting, providing a more ideal therapy profile, and cover a broader spectrum of patient population.

TABLE 3
Nonpharmacological interventions for the treatment of Depression

| | Regionally | Clinically | |
|---|---|---|---|
| Intervention | specific | applicable | Invasive |
| Transcranial | ++++ | +++ | + (painful at high |
| magnetic | | | intensities) |
| stimulation | | | |
| Vagus nerve | ++ | +++ | +++ (surgery for |
| stimulation | | | generator implant) |

As mentioned previously, any combination, or sequence, or time intervals of these two energies may be applied, and is considered within the scope of the invention.

In some patients the beneficial effects of rTMS may last for sometime. These patient's may be implanted with the vagus nerve stimulator sometime after receiving their last dose of rTMS therapy. Typically patients who have received TMS, and need a more aggressive therapy for treatment would be provided VNS. This form of combination therapy, where a patient receives rTMS therapy initially and sometime later receives pulsed electrical stimulation therapy, is also intended to be covered in the scope of the invention.

### ECT Used with Afferent Vagus Nerve Stimulation for Depression

Shown in conjunction with FIG. 25 were some advantages and disadvantages of various forms of nonpharmalogical interventions for the treatment of depression. As one example, ECT has clinical applicability in the short run, but on the other hand is associated with long-lasting cognitive impairments. Considering the advantages and disadvantages of different existing treatments, a combination of ECT therapy and pulsed electrical vagus nerve stimulation is an ideal combination for device based interventions, with or without concomitant drug therapy. Furthermore, in this unique combination, ECT induces stimulation from outside, and vagus nerve stimulation (VNS) approaches the stimulation of centers in brain from inside. Interestingly, electroconvulsive therapy (ECT) is found to decrease prefrontal rCBF according to the majority of studies.

Based on this thinking as shown in conjunction with Table 4, which highlights that ECT and vagus nerve stimulation are an ideal combination of nonpharmalogical interventions, with or without concomitant drug therapy.

TABLE 4
Nonpharmacological interventions for the treatment of Depression

| | Regionally | Clinically | |
|---|---|---|---|
| Intervention | specific | applicable | Invasive |
| Electroconvulsive | ++ (+++ if | ++++ | ++ (anesthesia, |
| therapy (ECT) | induced by | | generalized seizure) |
| | magnets) | | |
| Vagus nerve | ++ | +++ | +++ (surgery for |
| stimulation | | | generator implant) |

The initiation and delivery of these two interventions may be in any sequence or combination, and may be in addition to any drug therapy. For example, a patient implanted with vagal nerve stimulator may be given ECT therapy, or alternatively a patient receiving ECT therapy may be implanted with a vagus nerve stimulator. Of course, this may be in addition to any drug therapy that may be given to a patient. It is an object of this invention to provide an optimal device based therapy for depression by supplementing ECT with VNS. ECT provided alone usually has cognitive adverse effects. Advantageously, not only would the cognitive adverse effects be reduced, but the efficacy would also be significantly improved by the combination of ECT and VNS as disclosed in this application.

Applicant's co-pending application Ser. No. 11/086,526, entitled “Method and system to provide therapy for depression using electroconvulsive therapy (ECT) and pulsed electrical stimulation to vagus nerve(s)” is incorporated herein by reference.

### Pulse Generator Means

Many of the patients may end up with more than one type of pulse generator in their lifetime. In the methodology of this invention, an implanted lead has a terminal end which is compatible with different embodiments of pulse generators disclosed in this application. Once the lead is implanted in a patient, any embodiment of the pulse generator disclosed in this application, may be implanted in the patient. Furthermore, at replacement the same embodiment or a different embodiment may be implanted in the patient using the same lead. This may be repeated as long as the implanted lead is functional and maintains its integrity.

As one example, without limitation, an implanted stimulus-receiver in conjunction with an external stimulator may be used initially to test patient's response. At a later time, the pulse generator may be exchanged for a more elaborate implanted pulse generator (IPG) model, keeping the same lead. Some examples of stimulation and power sources that may be used for the practice of this invention, and disclosed in this application, include:

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a microstimulator;

e) a programmable implantable pulse generator;

f) a combination implantable device comprising both a stimulus-receiver and a programmable IPG; and

g) an IPG comprising a rechargeable battery.

All of these pulse generator means can generate and emit rectangular and complex electrical pulses. Complex electrical pulses comprise at least one of multi-level pulses, biphasic pulses, non-rectangular pulses, or pulses with varying amplitude during the pulse.

### Implanted Stimulus-Receiver with an External Stimulator

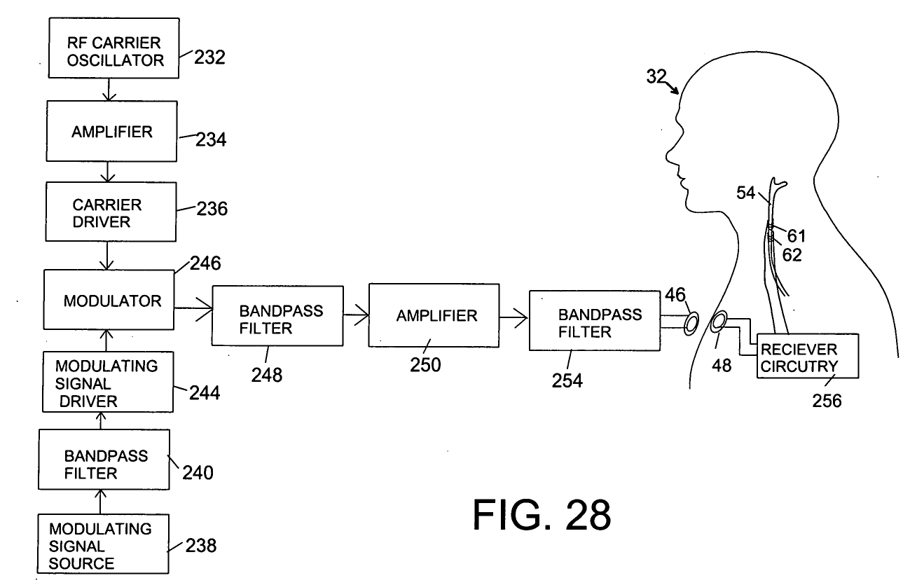

The selective stimulation of various nerve fibers of a cranial nerve such as the vagus nerve (or neuromodulation of the vagus nerve), as performed by one embodiment of the method and system of this invention is shown schematically in FIG. 28, as a block diagram. A modulator 246 receives analog (sine wave) high frequency “carrier” signal and modulating signal. The modulating signal can be multilevel digital, binary, or even an analog signal. In this embodiment, mostly multilevel digital type modulating signals are used. The modulated signal is amplified 250, conditioned 254, and transmitted via a primary coil 46 which is external to the body. A secondary coil 48 of an implanted stimulus receiver, receives, demodulates, and delivers these pulses to the vagus nerve 54 via electrodes 61 and 62. The receiver circuitry 256 is described later.

The carrier frequency is optimized. One preferred embodiment utilizes electrical signals of around 1 Mega-Hertz, even though other frequencies can be used. Low frequencies are generally not suitable because of energy requirements for longer wavelengths, whereas higher frequencies are absorbed by the tissues and are converted to heat, which again results in power losses.

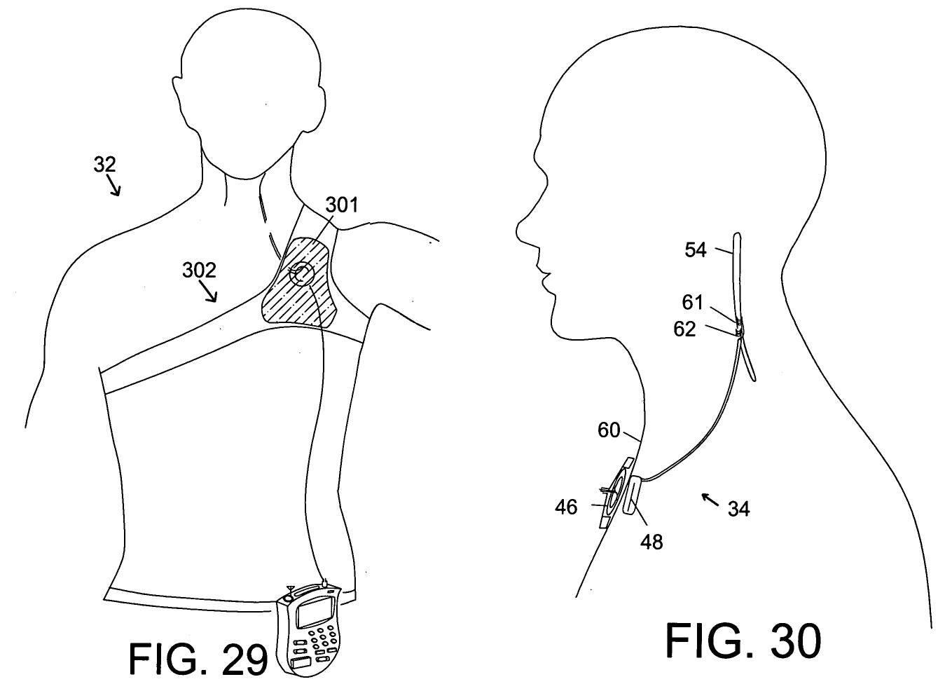

Shown in conjunction with FIG. 29, the coil for the external transmitter (primary coil 46) may be placed in the pocket 301 of a customized garment 302, for patient convenience.

Shown in conjunction with FIG. 30, the primary (external) coil 46 of the external stimulator 42 is inductively coupled to the secondary (implanted) coil 48 of the implanted stimulus-receiver 34. The implantable stimulus-receiver 34 has circuitry at the proximal end, and has two stimulating electrodes at the distal end 61,62. The negative electrode (cathode) 61 is positioned towards the brain and the positive electrode (anode) 62 is positioned away from the brain.

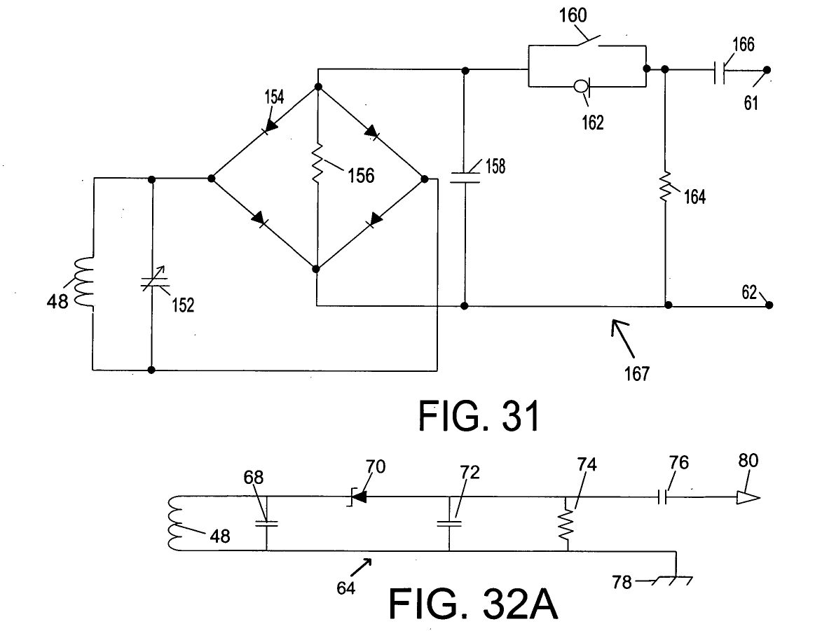

The circuitry contained in the proximal end of the implantable stimulus-receiver 34 is shown schematically in FIG. 31, for one embodiment. In this embodiment, the circuit uses all passive components. Approximately 25 turn copper wire of 30 gauge, or comparable thickness, is used for the primary coil 46 and secondary coil 48. This wire is concentrically wound with the windings all in one plane. The frequency of the pulse-waveform delivered to the implanted coil 48 can vary, and so a variable capacitor 152 provides ability to tune secondary implanted circuit 167 to the signal from the primary coil 46. The pulse signal from secondary (implanted) coil 48 is rectified by the diode bridge 154 and frequency reduction obtained by capacitor 158 and resistor 164. The last component in line is capacitor 166, used for isolating the output signal from the electrode wire. The return path of signal from cathode 61 will be through anode 62 placed in proximity to the cathode 61 for “Bipolar” stimulation. In this embodiment bipolar mode of stimulation is used, however, the return path can be connected to the remote ground connection (case) of implantable circuit 167, providing for much larger intermediate tissue for “Unipolar” stimulation. The “Bipolar” stimulation offers localized stimulation of tissue compared to “Unipolar” stimulation and is therefore, preferred in this embodiment. Unipolar stimulation is more likely to stimulate skeletal muscle in addition to nerve stimulation. The implanted circuit 167 in this embodiment is passive, so a battery does not have to be implanted.

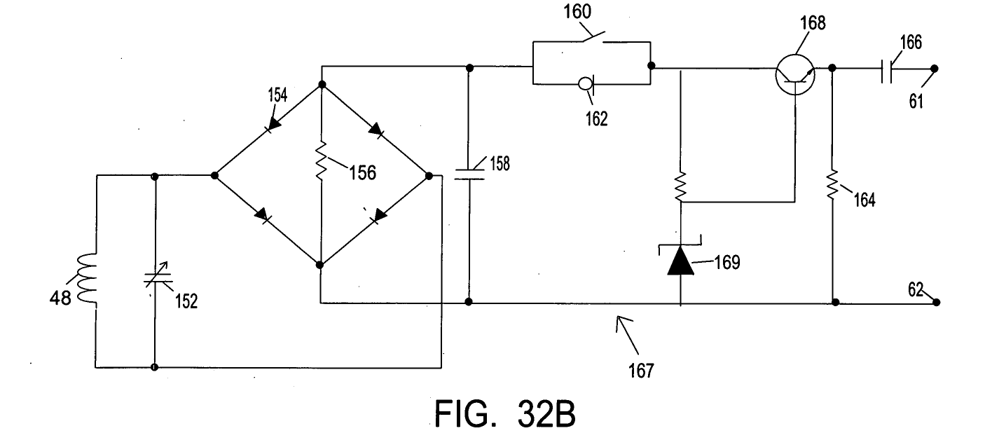

The circuitry shown in FIGS. 32A and 32B can be used as an alternative, for the implanted stimulus-receiver. The circuitry of FIG. 32A is a slightly simpler version, and circuitry of FIG. 32B contains a conventional NPN transistor 168 connected in an emitter-follower configuration.

For therapy to commence, the primary (external) coil 46 is placed on the skin 60 on top of the surgically implanted (secondary) coil 48. An adhesive tape is then placed on the skin 60 and external coil 46 such that the external coil 46, is taped to the skin 60. For efficient energy transfer to occur, it is important that the primary (external) and secondary (internal) coils 46,48 be positioned along the same axis and be optimally positioned relative to each other. In this embodiment, the external coil 46 may be connected to proximity sensing circuitry 50. The correct positioning of the external coil 46 with respect to the internal coil 48 is indicated by turning “on” of a light emitting diode (LED) on the external stimulator 42.

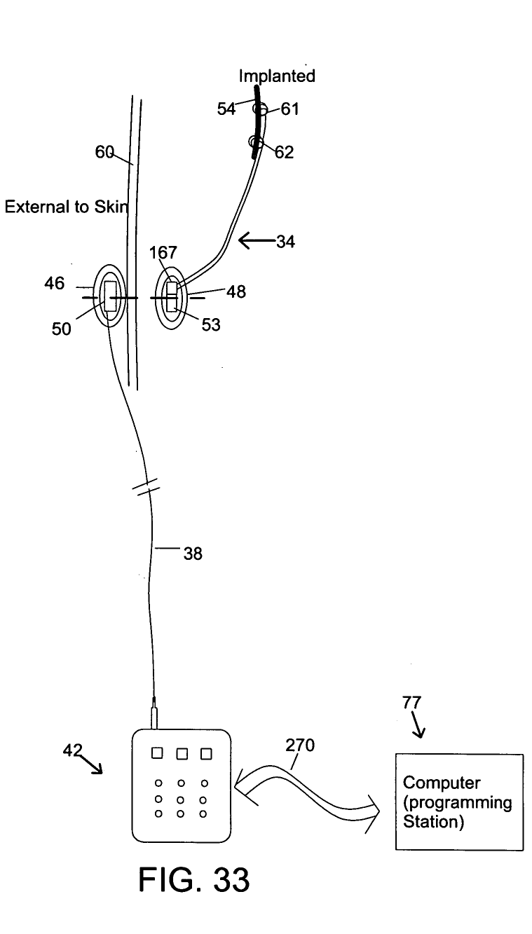

Optimal placement of the external (primary) coil 46 is done with the aid of proximity sensing circuitry incorporated in the system, in this embodiment. Proximity sensing occurs utilizing a combination of external and implantable components. The implanted components contains a relatively small magnet composed of materials that exhibit Giant Magneto-Resistor (GMR) characteristics such as Samarium-cobalt, a coil, and passive circuitry. Shown in conjunction with FIG. 33, the external coil 46 and proximity sensor circuitry 50 are rigidly connected in a convenient enclosure which is attached externally on the skin. The sensors measure the direction of the field applied from the magnet to sensors within a specific range of field strength magnitude. The dual sensors exhibit accurate sensing under relatively large separation between the sensor and the target magnet. As the external coil 46 placement is “fine tuned”, the condition where the external (primary) coil 46 comes in optimal position, i.e. is located adjacent and parallel to the subcutaneous (secondary) coil 48, along its axis, is recorded and indicated by a light emitting diode (LED) on the external stimulator 42.

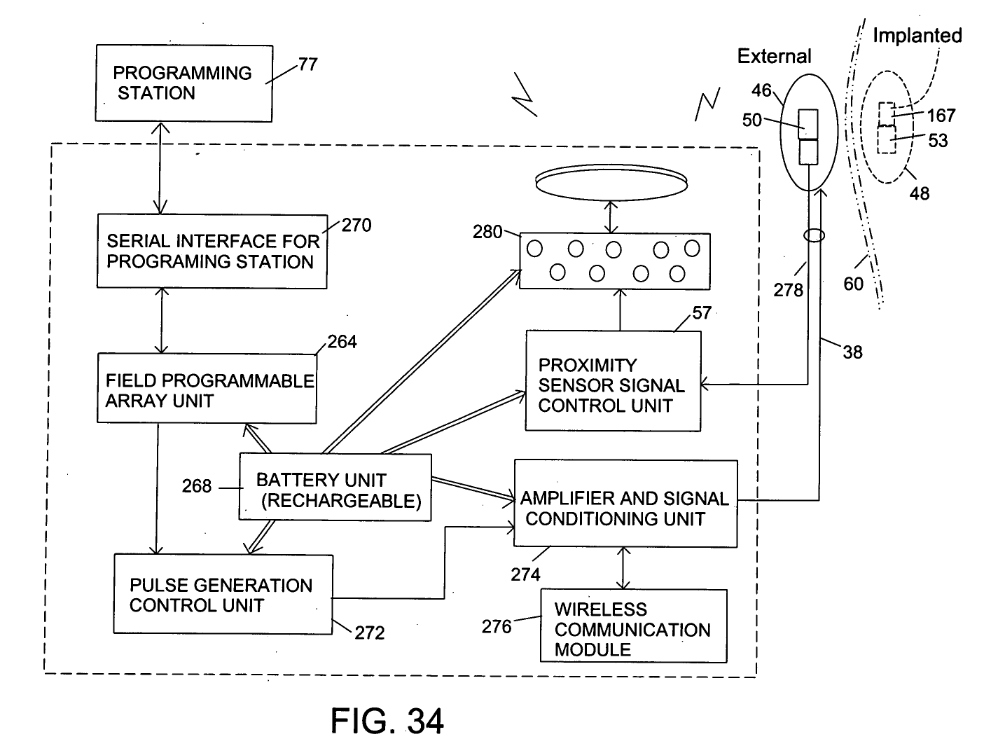

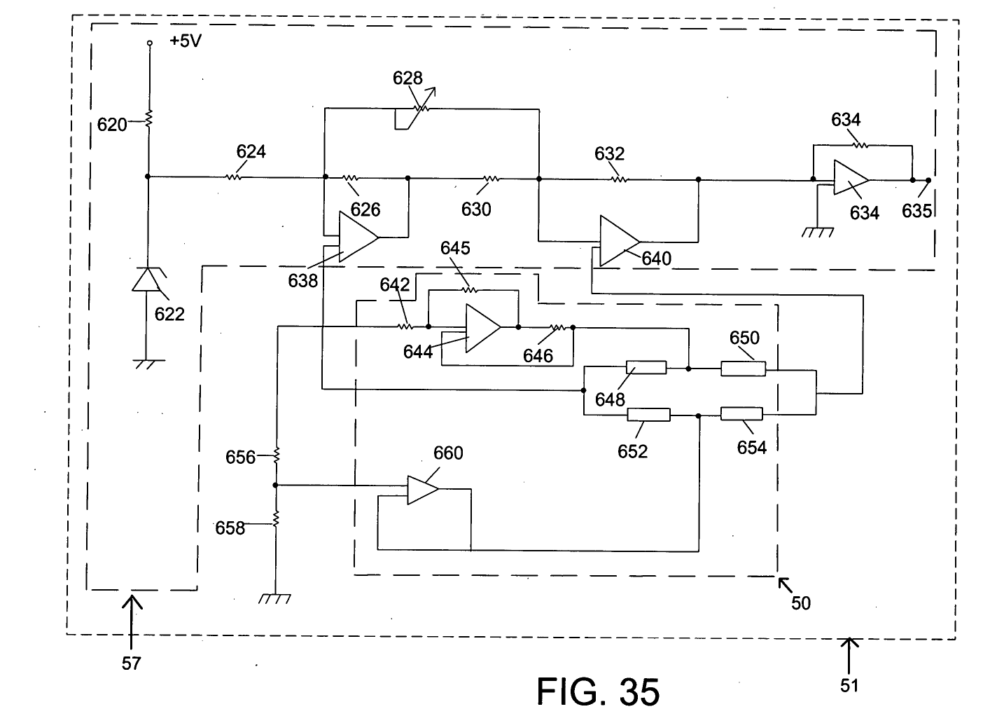

FIG. 34 shows an overall block diagram of the components of the external stimulator and the proximity sensing mechanism. The proximity sensing components are the primary (external) coil 46, supercutaneous (external) proximity sensors 648, 652 (FIG. 35) in the proximity sensor circuit unit 50, and a subcutaneous secondary coil 48 with a Giant Magneto Resister (GMR) magnet 53 associated with the proximity sensor unit. The proximity sensor circuit 50 provides a measure of the position of the secondary implanted coil 48. The signal output from proximity sensor circuit 50 is derived from the relative location of the primary and secondary coils 46, 48. The sub-assemblies consist of the coil and the associated electronic components, that are rigidly connected to the coil.

The proximity sensors (external) contained in the proximity sensor circuit 50 detect the presence of a GMR magnet 53, composed of Samarium Cobalt, that is rigidly attached to the implanted secondary coil 48. The proximity sensors, are mounted externally as a rigid assembly and sense the actual separation between the coils, also known as the proximity distance. In the event that the distance exceeds the system limit, the signal drops off and an alarm sounds to indicate failure of the production of adequate signal in the secondary implanted circuit 167, as applied in this embodiment of the device. This signal is provided to the location indicator LED 280.

FIG. 35 shows the circuit used to drive the proximity sensors 648, 652 of the proximity sensor circuit 50. The two proximity sensors 648, 652 obtain a proximity signal based on their position with respect to the implanted GMR magnet 53. This circuit also provides temperature compensation. The sensors 648, 652 are ‘Giant Magneto Resistor’ (GMR) type sensors packaged as proximity sensor unit 50. There are two components of the complete proximity sensor circuit. One component is mounted supercutaneously 50, and the other component, the proximity sensor signal control unit 57 is within the external stimulator 42. The resistance effect depends on the combination of the soft magnetic layer of magnet 53, where the change of direction of magnetization from external source can be large, and the hard magnetic layer, where the direction of magnetization remains unchanged. The resistance of this sensor 50 varies along a straight motion through the curvature of the magnetic field. A bridge differential voltage is suitably amplified and used as the proximity signal.

The Siemens GMR B6 (Siemens Corp., Special Components Inc., New Jersey) is used for this function in one embodiment. The maximum value of the peak-to-peak signal is observed as the external magnetic field becomes strong enough, at which point the resistance increases, resulting in the increase of the field-angle between the soft magnetic and hard magnetic material. The bridge voltage also increases. In this application, the two sensors 648, 652 are oriented orthogonal to each other.

The distance between the magnet 53 and sensor 50 is not relevant as long as the magnetic field is between 5 and 15 KA/m, and provides a range of distances between the sensors 648, 652 and the magnetic material 53. The GMR sensor registers the direction of the external magnetic field. A typical magnet to induce permanent magnetic field is approximately 15 by 8 by mm³, for this application and these components. The sensors 648, 652 are sensitive to temperature, such that the corresponding resistance drops as temperature increases. This effect is quite minimal until about 100° C. A full bridge circuit is used for temperature compensation, as shown in temperature compensation circuit 50 of FIG. 35. The sensors 648, 652 and a pair of resistors 650, 654 are shown as part of the bridge network for temperature compensation. It is also possible to use a full bridge network of two additional sensors in place of the resistors 650, 654.

The signal from either proximity sensor 648, 652 is rectangular if the surface of the magnetic material is normal to the sensor and is radial to the axis of a circular GMR device. This indicates a shearing motion between the sensor and the magnetic device. When the sensor is parallel to the vertical axis of this device, there is a fall off of the relatively constant signal at about 25 mm separation. The GMR sensor combination varies its resistance according to the direction of the external magnetic field, thereby providing an absolute angle sensor. The position of the GMR magnet can be registered at any angle from 0 to 360 degrees.

In the external stimulator 42 shown in FIG. 34, an indicator unit 280 which is provided to indicate proximity distance or coil proximity failure (for situations where the patch containing the external coil 46, has been removed, or is twisted abnormally etc.). Indication is also provided to assist in the placement of the patch. In case of general failure, a red light with audible signal is provided when the signal is not reaching the subcutaneous circuit. The indicator unit 280 also displays low battery status. The information on the low battery, normal and out of power conditions forewarns the user of the requirements of any corrective actions.

Also shown in FIG. 34, the programmable parameters are stored in a programmable logic 264. The predetermined programs stored in the external stimulator are capable of being modified through the use of a separate programming station 77. The Programmable Array Logic Unit 264 and interface unit 270 are interfaced to the programming station 77. The programming station 77 can be used to load new programs, change the existing predetermined programs or the program parameters for various stimulation programs. The programming station is connected to the programmable array unit 75 (comprising programmable array logic 304 and interface unit 270) with an RS232-C serial connection. The main purpose of the serial line interface is to provide an RS232-C standard interface. Other suitable connectors such as a USB connector or other connectors with standard protocols may also be used.

This method enables any portable computer with a serial interface to communicate and program the parameters for storing the various programs. The serial communication interface receives the serial data, buffers this data and converts it to a 16 bit parallel data. The programmable array logic 264 component of programmable array unit receives the parallel data bus and stores or modifies the data into a random access matrix. This array of data also contains special logic and instructions along with the actual data. These special instructions also provide an algorithm for storing, updating and retrieving the parameters from long-term memory. The programmable logic array unit 264, interfaces with long term memory to store the predetermined programs. All the previously modified programs can be stored here for access at any time, as well as, additional programs can be locked out for the patient. The programs consist of specific parameters and each unique program will be stored sequentially in long-term memory. A battery unit is present to provide power to all the components. The logic for the storage and decoding is stored in a random addressable storage matrix (RASM).

Conventional microprocessor and integrated circuits are used for the logic, control and timing circuits. Conventional bipolar transistors are used in radio-frequency oscillator, pulse amplitude ramp control and power amplifier. A standard voltage regulator is used in low-voltage detector. The hardware and software to deliver the pre-determined programs is well known to those skilled in the art.

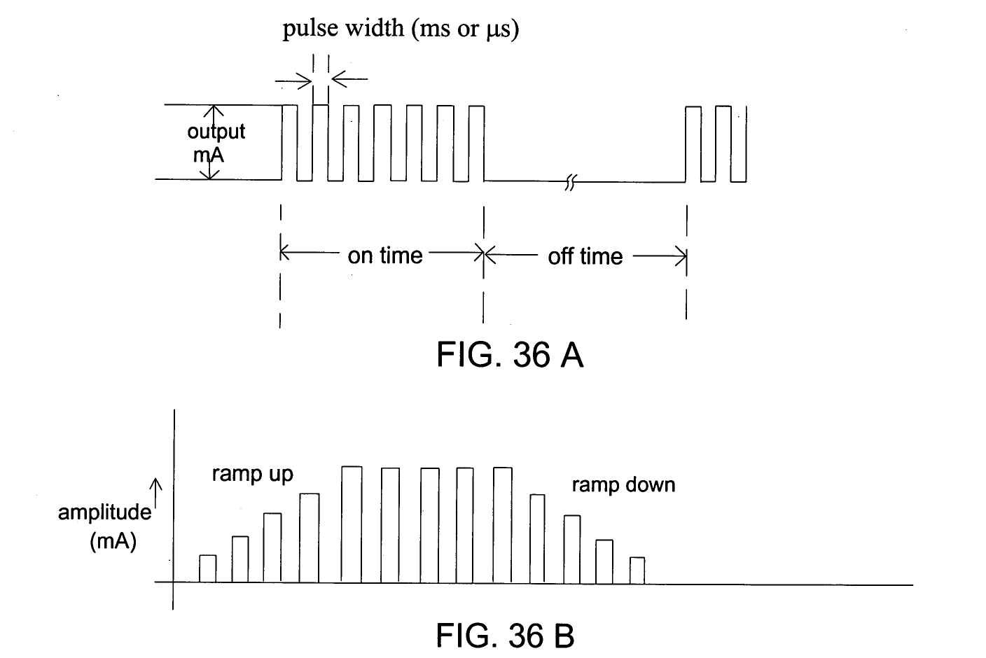

The pulses delivered to the nerve tissue for stimulation therapy are shown graphically in FIG. 36A. As shown in FIG. 36B, for patient comfort when the electrical stimulation is turned on, the electrical stimulation is ramped up and ramped down, instead of abrupt delivery of electrical pulses.

The selective stimulation to the vagus nerve can be performed in one of two ways. One method is to activate one of several “pre-determined” programs. A second method is to “custom” program the electrical parameters which can be selectively programmed, for specific therapy to the individual patient. The electrical parameters which can be individually programmed, include variables such as pulse amplitude, pulse width, frequency of stimulation, stimulation on-time, and stimulation off-time. Table three below defines the approximate range of parameters,

TABLE 3
Electrical parameter range delivered to the nerve

| PARAMER | RANGE |
|---|---|
| Pulse Amplitude | 0.1 Volt-15 Volts |
| Pulse width | 20 μS-5 mSec. |
| Stim. Frequency | 5 Hz-200 Hz |
| Freq. for blocking | DC to 750 Hz |
| On-time | 5 Secs-24 hours |
| Off-time | 5 Secs-24 hours |

The parameters in Table 3 are the electrical signals delivered to the nerve via the two electrodes 61,62 (distal and proximal) around the nerve, as shown in FIG. 30. It being understood that the signals generated by the external pulse generator 42 and transmitted via the primary coil 46 are larger, because the attenuation factor between the primary coil and secondary coil is approximately 10-20 times, depending upon the distance, and orientation between the two coils. Accordingly, the range of transmitted signals of the external pulse generator are approximately 10-20 times larger than shown in Table 2.

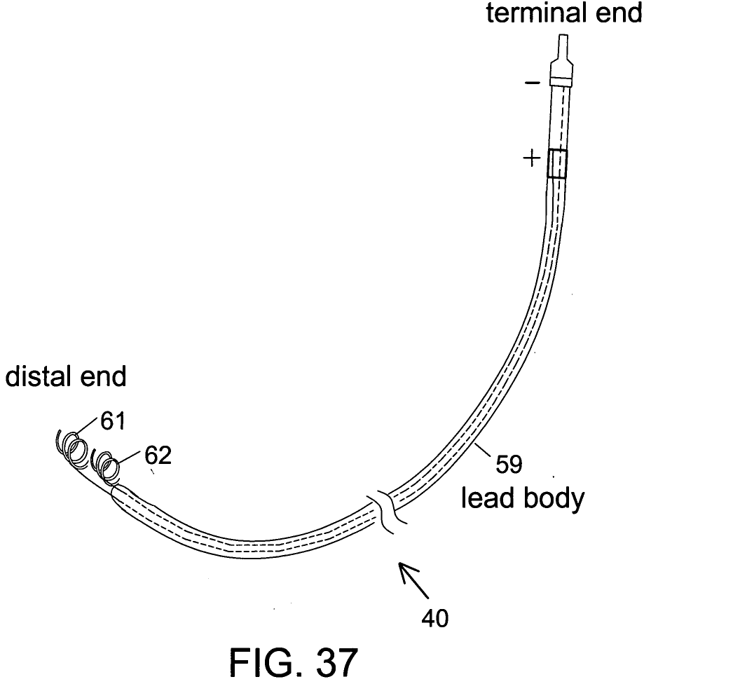

Referring now to FIG. 37, the implanted lead 40 component of the system is similar to cardiac pacemaker leads, except for distal portion (or electrode end) of the lead. The lead terminal preferably is linear bipolar, even though it can be bifurcated, and plug(s) into the cavity of the pulse generator means. The lead body 59 insulation may be constructed of medical grade silicone, silicone reinforced with polytetrafluoro-ethylene (PTFE), or polyurethane. The electrodes 61,62 for stimulating the vagus nerve 54 may either wrap around the nerve once or may be spiral shaped. These stimulating electrodes may be made of pure platinum, platinum/Iridium alloy or platinum/iridium coated with titanium nitride. The conductor connecting the terminal to the electrodes 61,62 is made of an alloy of nickel-cobalt. The implanted lead design variables are also summarized in table four below.

TABLE 4
Lead design variables

| | |
|---|---|
| Proximal | Distal |
| End | End |

| | | | Conductor | | |
|---|---|---|---|---|---|
| | Lead body- | | (connecting | | |
| Lead | Insulation | | proximal and | Electrode - | Electrode - |
| Terminal | Materials | Lead-Coating | distal ends) | Material | Type |
| Linear | Polyurethane | Antimicrobial | Alloy of | Pure Platinum | Spiral |
| bipolar | | coating | Nickel-Cobalt | | electrode |
| Bifurcated | Silicone | Anti-Inflammatory | | Platinum-Iridium | Wrap-around |
| | | coating | | (Pt/Ir) Alloy | electrode |
| | Silicone with | Lubricious | | Pt/Ir coated | Steroid |
| | Polytetra- | coating | | with Titanium | eluting |
| | fluoroethylene | | | Nitride | |
| | (PTFE) | | | | |
| | | | | Carbon | Hydrogel |
| | | | | | electrodes |
| | | | | | Cuff |
| | | | | | electrodes |

Examples of electrode designs are also shown in U.S. Pat. No. 5,215,089 (Baker), U.S. Pat. No. 5,351,394 (Weinburg), and U.S. Pat. No. 6,600,956 (Mashino), which are incorporated herein by reference.

Once the lead is fabricated, coating such as anti-microbial, anti-inflammatory, or lubricious coating may be applied to the body of the lead.

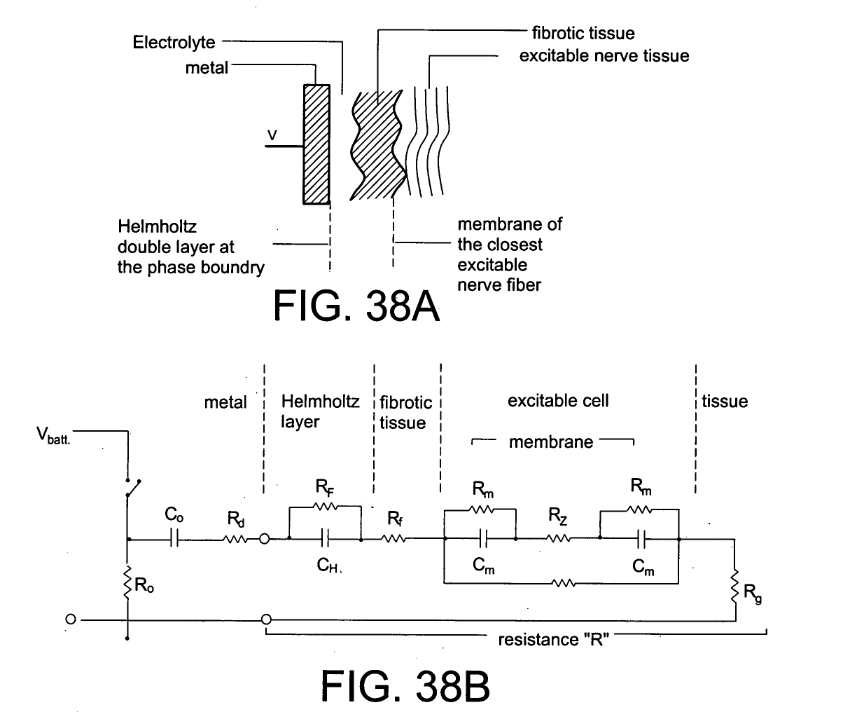

FIG. 38A summarizes electrode-tissue interface between the nerve tissue and electrodes 61, 62. There is a thin layer of fibrotic tissue between the stimulating electrode 61 and the excitable nerve fibers of the vagus nerve 54. FIG. 38B summarizes the most important properties of the metal/tissue phase boundary in an equivalent circuit diagram. Both the membrane of the nerve fibers and the electrode surface are represented by parallel capacitance and resistance. Application of a constant battery voltage Vbat from the pulse generator, produces voltage changes and current flow, the time course of which is crucially determined by the capacitive components in the equivalent circuit diagram. During the pulse, the capacitors Co, Ch and Cm are charged through the ohmic resistances, and when the voltage Vbat is turned off, the capacitors discharge with current flow on the opposite direction.

### Implanted Stimulus-Receiver Comprising a High Value Capacitor for Storing Charge, Used in Conjunction with an External Stimulator

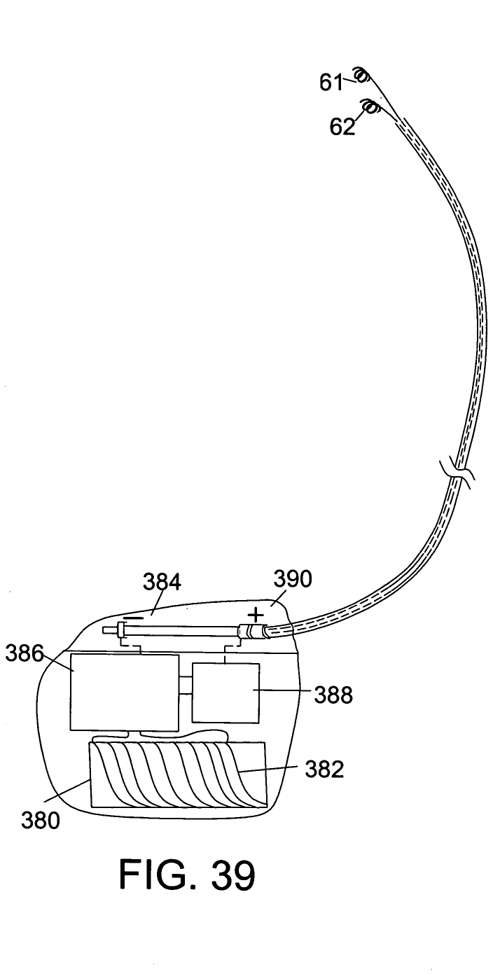

In one embodiment, the implanted stimulus-receiver may be a system which is RF coupled combined with a power source. In this embodiment, the implanted stimulus-receiver contains high value, small sized capacitor(s) for storing charge and delivering electric stimulation pulses for up to several hours by itself, once the capacitors are charged. The packaging is shown in FIG. 39. Using mostly hybrid components and appropriate packaging, the implanted portion of the system described below is conducive to miniaturization. As shown in FIG. 29, a solenoid coil 382 wrapped around a ferrite core 380 is used as the secondary of an air-gap transformer for receiving power and data to the implanted device. The primary coil is external to the body. Since the coupling between the external transmitter coil and receiver coil 382 may be weak, a high-efficiency transmitter/amplifier is used in order to supply enough power to the receiver coil 382. Class-D or Class-E power amplifiers may be used for this purpose. The coil for the external transmitter (primary coil) may be placed in the pocket of a customized garment.

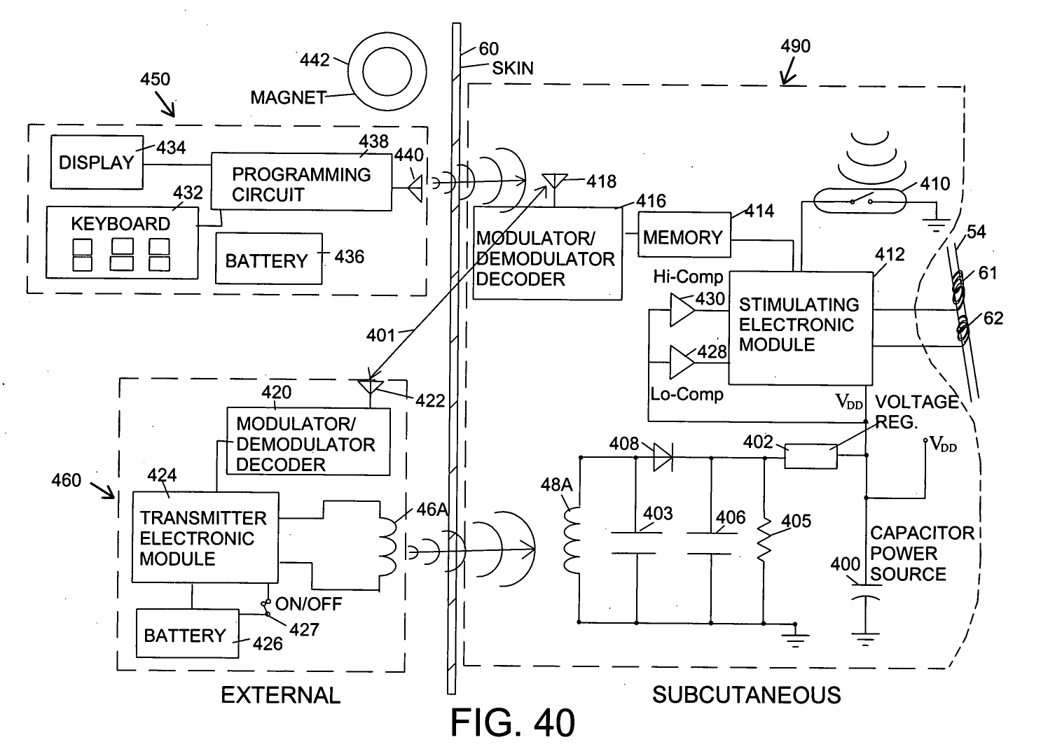

As shown in conjunction with FIG. 40 of the implanted stimulus-receiver 490 and the system, the receiving inductor 48A and tuning capacitor 403 are tuned to the frequency of the transmitter. The diode 408 rectifies the AC signals, and a small sized capacitor 406 is utilized for smoothing the input voltage V_{I} fed into the voltage regulator 402. The output voltage V_{D} of regulator 402 is applied to capacitive energy power supply and source 400 which establishes source power V_{DD}. Capacitor 400 is a big value, small sized capacative energy source which is classified as low internal impedance, low power loss and high charge rate capacitor, such as Panasonic Model No. 641.

The refresh-recharge transmitter unit 460 includes a primary battery 426, an ON/Off switch 427, a transmitter electronic module 442, an RF inductor power coil 46A, a modulator/demodulator 420 and an antenna 422.

When the ON/OFF switch is on, the primary coil 46A is placed in close proximity to skin 60 and secondary coil 48A of the implanted stimulator 490. The inductor coil 46A emits RF waves establishing EMF wave fronts which are received by secondary inductor 48A. Further, transmitter electronic module 442 sends out command signals which are converted by modulator/demodulator decoder 420 and sent via antenna 422 to antenna 418 in the implanted stimulator 490. These received command signals are demodulated by decoder 416 and replied and responded to, based on a program in memory 414 (matched against a “command table” in the memory). Memory 414 then activates the proper controls and the inductor receiver coil 48A accepts the RF coupled power from inductor 46A.

The RF coupled power, which is alternating or AC in nature, is converted by the rectifier 408 into a high DC voltage. Small value capacitor 406 operates to filter and level this high DC voltage at a certain level. Voltage regulator 402 converts the high DC voltage to a lower precise DC voltage while capacitive power source 400 refreshes and replenishes.

When the voltage in capacative source 400 reaches a predetermined level (that is V_{DD} reaches a certain predetermined high level), the high threshold comparator 430 fires and stimulating electronic module 412 sends an appropriate command signal to modulator/decoder 416. Modulator/decoder 416 then sends an appropriate “fully charged” signal indicating that capacitive power source 400 is fully charged, is received by antenna 422 in the refresh-recharge transmitter unit 460.

In one mode of operation, the patient may start or stop stimulation by waving the magnet 442 once near the implant. The magnet emits a magnetic force Lₘ which pulls reed switch 410 closed. Upon closure of reed switch 410, stimulating electronic module 412 in conjunction with memory 414 begins the delivery (or cessation as the case may be) of controlled electronic stimulation pulses to the vagus nerve 54 via electrodes 61, 62. In another mode (AUTO), the stimulation is automatically delivered to the implanted lead based upon programmed ON/OFF times.

The programmer unit 450 includes keyboard 432, programming circuit 438, rechargeable battery 436, and display 434. The physician or medical technician programs programming unit 450 via keyboard 432. This program regarding the frequency, pulse width, modulation program, ON time etc. is stored in programming circuit 438. The programming unit 450 must be placed relatively close to the implanted stimulator 490 in order to transfer the commands and programming information from antenna 440 to antenna 418. Upon receipt of this programming data, modulator/demodulator and decoder 416 decodes and conditions these signals, and the digital programming information is captured by memory 414. This digital programming information is further processed by stimulating electronic module 412. In the DEMAND operating mode, after programming the implanted stimulator, the patient turns ON and OFF the implanted stimulator via hand held magnet 442 and the reed switch 410. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

### Programmer-Less Implantable Pulse Generator (IPG)

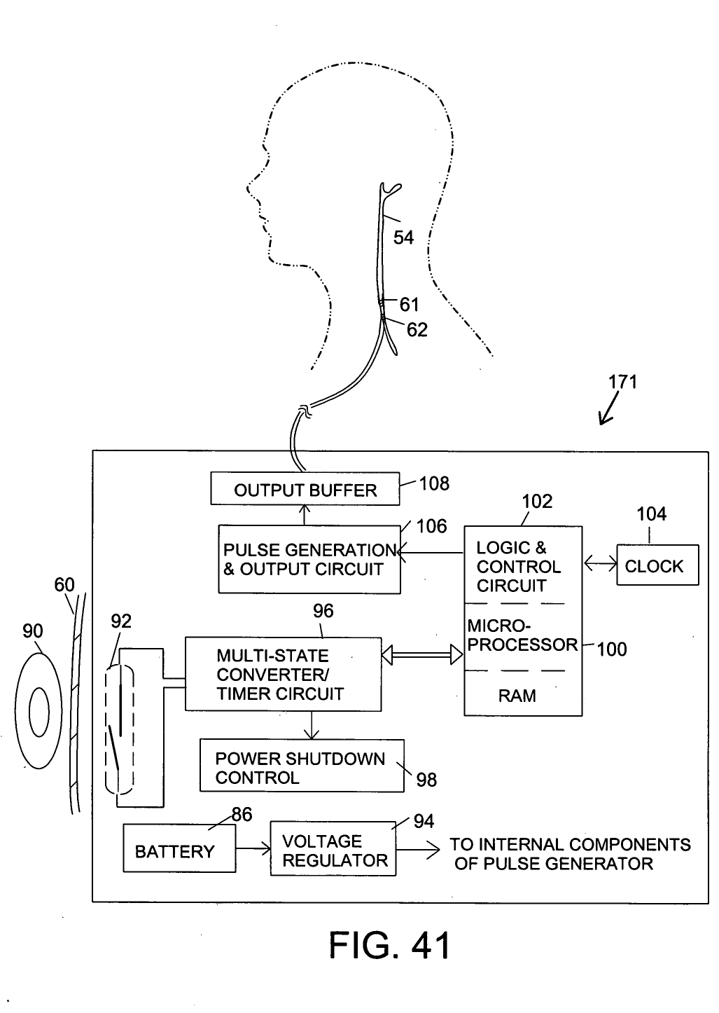

In one embodiment, a programmer-less implantable pulse generator (IPG) may be used, as disclosed in applicant's commonly assigned U.S. Pat. No. 6,760,626 B1, which is incorporated herein by reference. In this embodiment, shown in conjunction with FIG. 41, the implantable pulse generator 171 is provided with a reed switch 92 and memory circuitry 102. The reed switch 92 being remotely actuable by means of a magnet 90 brought into proximity of the pulse generator 171, in accordance with common practice in the art. In this embodiment, the reed switch 92 is coupled to a multi-state converter/timer circuit 96, such that a single short closure of the reed switch can be used as a means for non-invasive encoding and programming of the pulse generator 171 parameters.

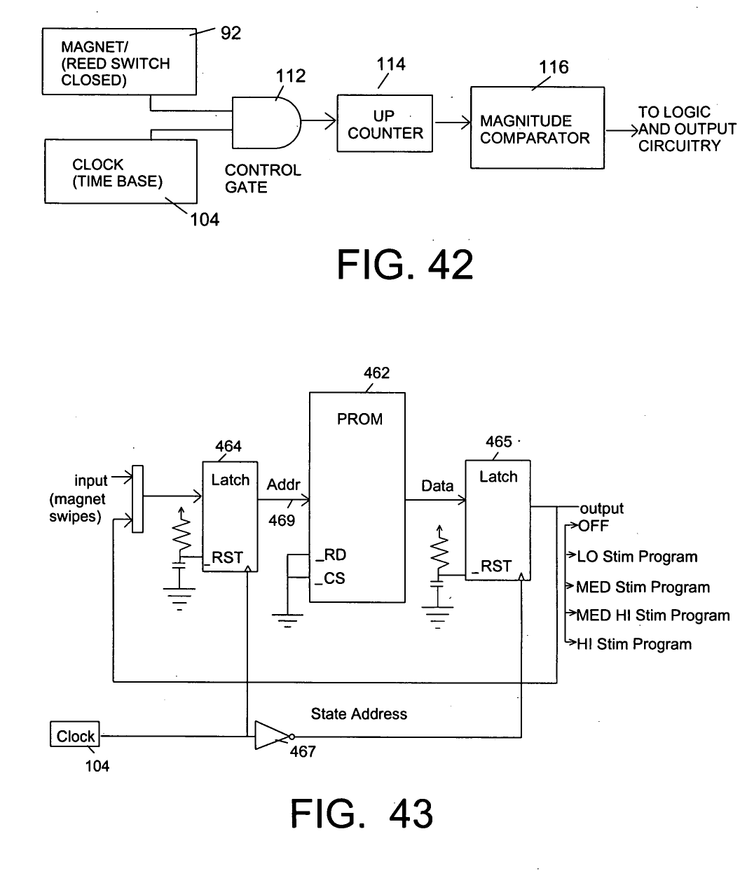

In one embodiment, shown in conjunction with FIG. 42, the closing of the reed switch 92 triggers a counter. The magnet 90 and timer are ANDed together. The system is configured such that during the time that the magnet 82 is held over the pulse generator 171, the output level goes from LOW stimulation state to the next higher stimulation state every 5 seconds. Once the magnet 82 is removed, regardless of the state of stimulation, an application of the magnet, without holding it over the pulse generator 171, triggers the OFF state, which also resets the counter.

Once the prepackaged/predetermined logic state is activated by the logic and control circuit 102, as shown in FIG. 41, the pulse generation and amplification circuit 106 deliver the appropriate electrical pulses to the vagus nerve 54 of the patient via an output buffer 108. The delivery of output pulses is configured such that the distal electrode 61 (electrode closer to the brain) is the cathode and the proximal electrode 62 is the anode. Timing signals for the logic and control circuit 102 of the pulse generator 171 are provided by a crystal oscillator 104. The battery 86 of the pulse generator 171 has terminals connected to the input of a voltage regulator 94. The regulator 94 smoothes the battery output and supplies power to the internal components of the pulse generator 171. A microprocessor 100 controls the program parameters of the device, such as the voltage, pulse width, frequency of pulses, on-time and off-time. The microprocessor may be a commercially available, general purpose microprocessor or microcontroller, or may be a custom integrated circuit device augmented by standard RAM/ROM components.

In one embodiment, there are four stimulation states. A larger (or lower) number of states can be achieved using the same methodology, and such is considered within the scope of the invention. These four states are, LOW stimulation state, LOW-MED stimulation state, MED stimulation state, and HIGH stimulation state. Examples of stimulation parameters (delivered to the vagus nerve) for each state are as follows,

LOW stimulation state example is,
Current output: 0.75 milliAmps.
Pulse width: 0.20 msec.
Pulse frequency: 20 Hz
Cycles: 20 sec. on-time and 2.0 min. off-time in repeating cycles.

LOW-MED stimulation state example is,
Current output: 1.5 milliAmps,
Pulse width: 0.30 msec.
Pulse frequency: 25 Hz
Cycles: 1.5 min. on-time and 20.0 min. off-time in repeating cycles.

MED stimulation state example is,
Current output: 2.0 milliAmps.
Pulse width: 0.30 msec.
Pulse frequency: 30 Hz
Cycles: 1.5 min. on-time and 20.0 min. off-time in repeating cycles.

HIGH stimulation state example is,
Current output: 3.0 milliAmps,
Pulse width: 0.40 msec.
Pulse frequency: 30 Hz
Cycles: 2.0 min. on-time and 20.0 min. off-time in repeating cycles.

These prepackaged/predetermined programs are mearly examples, and the actual stimulation parameters will deviate from these depending on the treatment application.

It will be readily apparent to one skilled in the art, that other schemes can be used for the same purpose. For example, instead of placing the magnet 90 on the pulse generator 171 for a prolonged period of time, different stimulation states can be encoded by the sequence of magnet applications. Accordingly, in an alternative embodiment there can be three logic states, OFF, LOW stimulation (LS) state, and HIGH stimulation (HS) state. Each logic state again corresponds to a prepackaged/predetermined program such as presented above. In such an embodiment, the system could be configured such that one application of the magnet triggers the generator into LS State. If the generator is already in the LS state then one application triggers the device into OFF State. Two successive magnet applications triggers the generator into MED stimulation state, and three successive magnet applications triggers the pulse generator in the HIGH Stimulation State. Subsequently, one application of the magnet while the device is in any stimulation state, triggers the device OFF.

FIG. 43 shows a representative digital circuitry used for the basic state machine circuit. The circuit consists of a PROM 462 that has part of its data fed back as a state address. Other address lines 469 are used as circuit inputs, and the state machine changes its state address on the basis of these inputs. The clock 104 is used to pass the new address to the PROM 462 and then pass the output from the PROM 462 to the outputs and input state circuits. The two latches 464, 465 are operated 180° out of phase to prevent glitches from unexpectedly affecting any output circuits when the ROM changes state. Each state responds differently according to the inputs it receives.

The advantage of this embodiment is that it is cheaper to manufacture than a fully programmable implantable pulse generator (IPG).

### Microstimulator

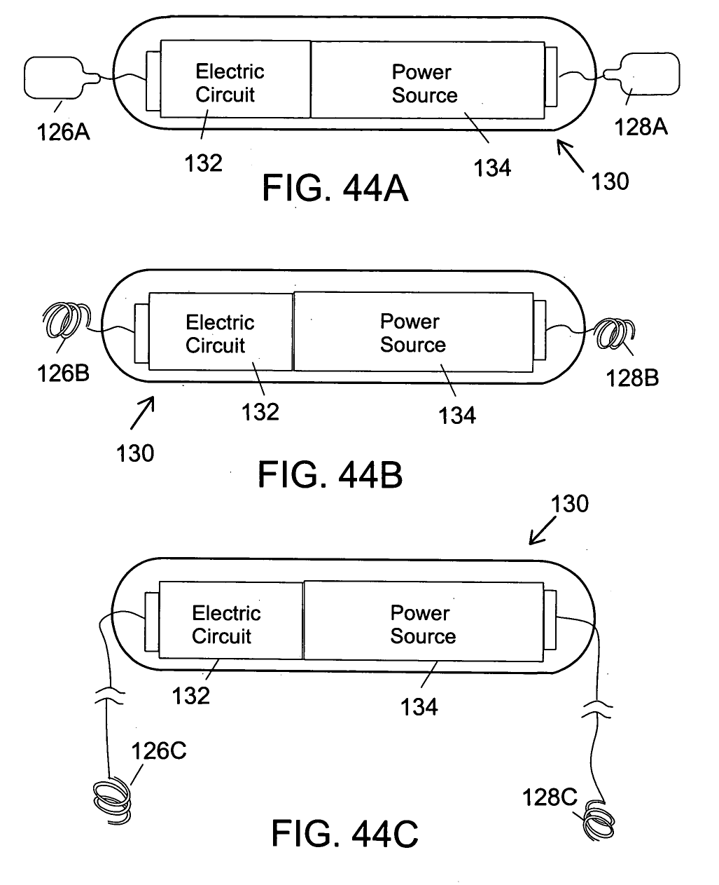

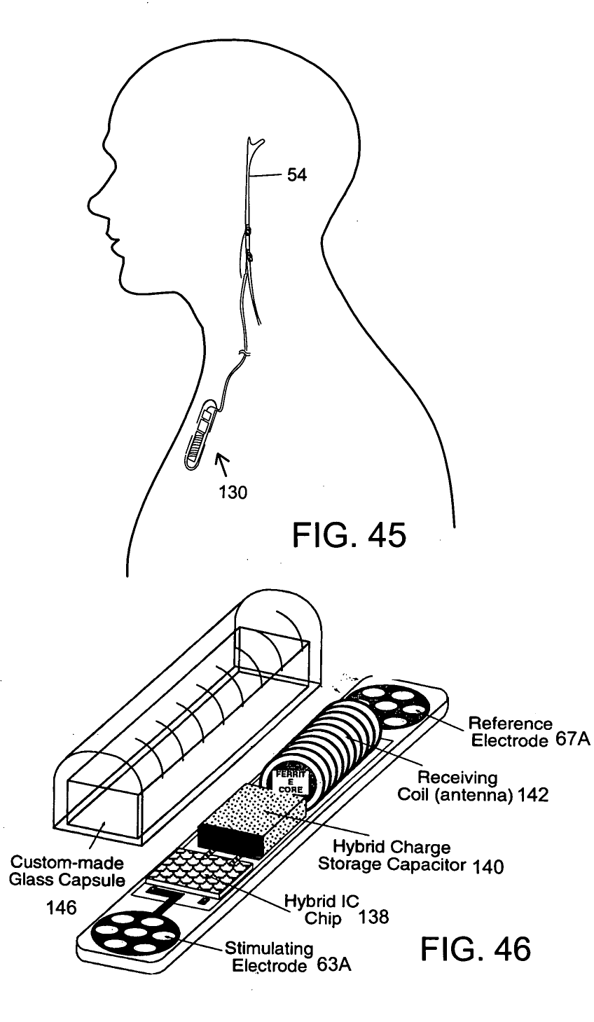

In one embodiment, a microstimulator 130 may be used for providing pulses to the vagus nerve(s) 54. Shown in conjunction with FIG. 44A, is a microstimulator where the electrical circuitry 132 and power source 134 are encased in a miniature hermetically sealed enclosure, and only the electrodes 126A, 128A are exposed. FIG. 44B depicts the same microstimulator, except the electrodes are modified and adapted to wrap around the nerve tissue 54. Because of its small size, the whole microstimulator may be in the proximity of the nerve tissue to be stimulated, or alternatively as shown in-conjunction with FIG. 45, the microstimulator may be implanted at a different site, and connected to the electrodes via conductors insulated with silicone and polyurethane (FIG. 44C).

Shown in reference with FIG. 46 is the overall structure of an implantable microstimulator 130. It consists of a micromachined silicon substrate that incorporates two stimulating electrodes which are the cathode and anode of a bipolar stimulating electrode pair 126A, 128A; a hybrid-connected tantalum chip capacitor 140 for power storage; a receiving coil 142; a bipolar-CMOS integrated circuit chip 138 for power regulation and control of the microstimulator; and a custom made glass capsule 146 that is electrostatically bonded to the silicon carrier to provide a hermetic package for the receiver-stimulator circuitry and hybrid elements. The stimulating electrode pair 63,64 resides outside of the package and feedthroughs are used to connect the internal electronics to the electrodes.

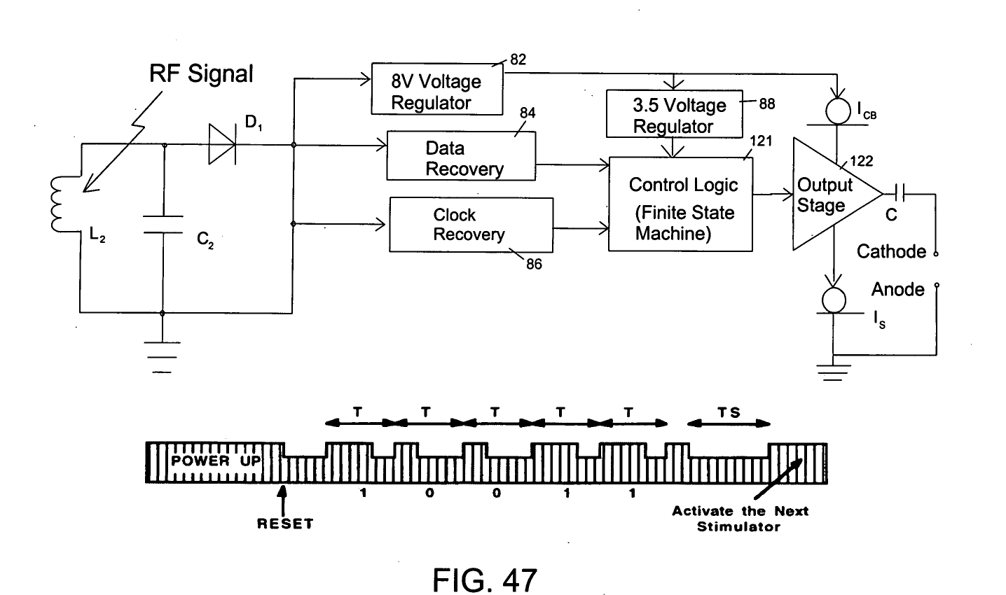

FIG. 47 shows the overall system electronics required for the microstimulator, and the power and data transmission protocol used for radiofrequency telemetry. The circuit receives an amplitude modulated RF carrier from an external transmitter and generates 8-V and 4-V dc supplies, generates a clock from the carrier signal, decodes the modulated control data, interprets the control data, and generates a constant current output pulse when appropriate. The RF carrier used for the telemetry link has a nominal frequency of around 1.8 MHz, and is amplitude modulated to encode control data. Logical “1” and “0” are encoded by varying the width of the amplitude modulated carrier, as shown in the bottom portion of FIG. 47. The carrier signal is initially high when the transmitter is turned on and sets up an electromagnetic field inside the transmitter coil. The energy in the field is picked up by receiver coils 142, and is used to charge the hybrid capacitor 140. The carrier signal is turned high and then back down again, and is maintained at the low level for a period between 1-200 μsec. The microstimulator 130 will then deliver a constant current pulse into the nerve tissue through the stimulating electrode pair 126A, 128A for the period that the carrier is low. Finally, the carrier is turned back high again, which will indicate the end of the stimulation period to the microstimulator 130, thus allowing it to charge its capacitor 140 back up to the on-chip voltage supply.

On-chip circuitry has been designed to generate two regulated power supply voltages (4V and 8V) from the RF carrier, to demodulate the RF carrier in order to recover the control data that is used to program the microstimulator, to generate the clock used by the on-chip control circuitry, to deliver a constant current through a controlled current driver into the nerve tissue, and to control the operation of the overall circuitry using a low-power CMOS logic controller.

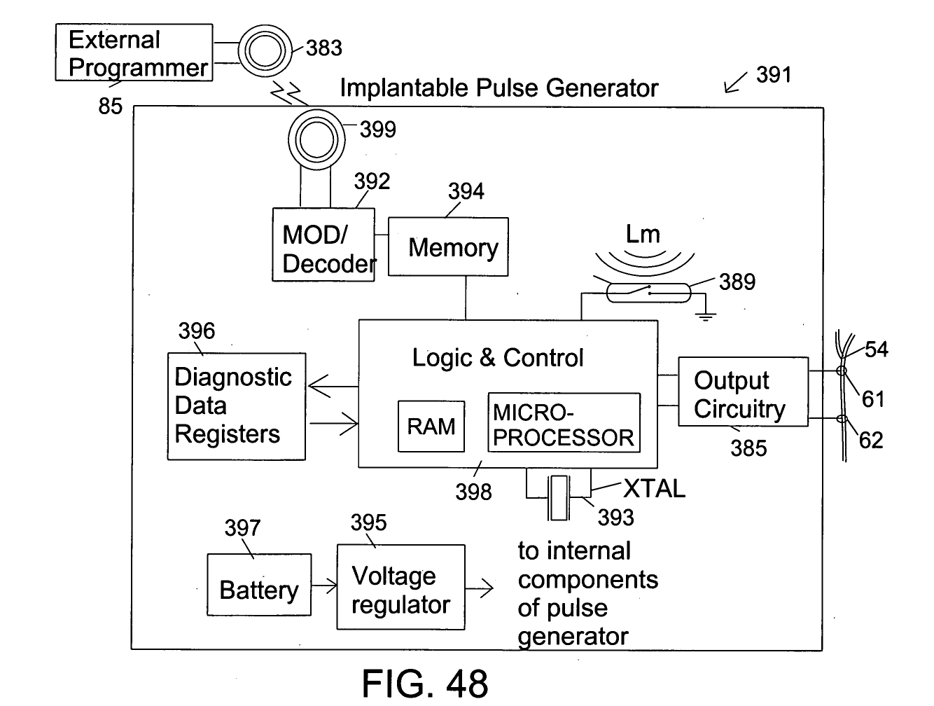

Programmable implantable pulse generator (IPG) In one embodiment, a fully programmable implantable pulse generator (IPG), capable of generating stimulation and blocking pulses may be used. Shown in conjunction with FIG. 48, the implantable pulse generator unit 391 is preferably a microprocessor based device, where the entire circuitry is encased in a hermetically sealed titanium can. As shown in the overall block diagram, the logic & control unit 398 provides the proper timing for the output circuitry 385 to generate electrical pulses that are delivered to electrodes 61, 62 via a lead 40. Programming of the implantable pulse generator (IPG) is done via an external programmer 85, as described later. Once activated or programmed via an external programmer 85, the implanted pulse generator 391 provides appropriate electrical stimulation pulses to the vagus nerve(s) 54 via electrodes 61,62.

This embodiment also comprises predetermined/pre-packaged programs. Examples of four stimulation states were given in the previous section, under “Programmer-less Implantable Pulse Generator (IPG)”. These predetermined/pre-packaged programs comprise unique combinations of pulse amplitude, pulse width, pulse morphology, pulse frequency, ON-time and OFF-time. Any number of predetermined/pre-packaged programs, even 100, can be stored in the implantable pulse generator of this invention, and are considered within the scope of the invention.

Examples of additional predetermined/pre-packaged programs are:

Program One:
Current output: 1.0 milliAmps.
Pulse width: 0.25 msec.
Pulse frequency: 20 Hz
Cycles: 20 sec. on-time and 3.0 min. off-time in repeating cycles.

Program Two:
Current output: 1.5 milliAmps,
Pulse width: 0.40 msec.
Pulse frequency: 25 Hz
Cycles: 3.0 min. on-time and 20.0 min. off-time in repeating cycles.

Program Three:
Current output: 2.0 milliAmps.
Pulse width: 0.50 msec.
Pulse frequency: 30 Hz
Cycles: 4 min. on-time and 20.0 min. off-time in repeating cycles.

Program Four:
Current output: 2.5 milliAmps,
Pulse width: 0.3 msec.
Pulse frequency: 25 Hz
Cycles: 4.0 min. on-time and 20.0 min. off-time in repeating cycles.

Program Five:
Current output: 3.0 milliAmps,
Pulse width: 0.50 msec.
Pulse frequency: 30 Hz
Cycles: 5.0 min. on-time and 30.0 min. off-time in repeating cycles.

Program Six (Fast Cycle):
Current output: 1.0 milliAmps.
Pulse width: 0.25 msec.
Pulse frequency: 20 Hz
Cycles: 8 sec. on-time and 12 sec. off-time in repeating cycles.

Program Seven (Fast Cycle):
Current output: 1.75 milliAmps.
Pulse width: 0.4 msec.
Pulse frequency: 30 Hz
Cycles: 8 sec. on-time and 12 sec. off-time in repeating cycles.

Program Eight (Complex Pulses):
Current output: 1.5 milliAmps.
Pulse width: 0.25 msec.
Pulse frequency: 25 Hz
Pulse type: step pulses
Cycles: 20 sec. on-time and 3.0 min. off-time in repeating cycles.

Program Nine (Complex Pulses):
Current output: 2.0 milliAmps.
Pulse width: 0.40 msec.
Pulse frequency: 30 Hz
Pulse type: step pulses
Cycles: 20 sec. on-time and 3.0 min. off-time in repeating cycles.

Program Ten (Complex Pulse Train):
Current output: 1.5 milliAmps.
Pulse width: 0.25 msec.
Pulse frequency: 25 Hz
Pulse type: step pulses with alternating pulse train (as shown in FIG. 46H)
Cycles: 20 sec. on-time and 3.0 min. off-time in repeating cycles.

These prepackaged/predetermined programs are mearly examples, and the actual stimulation parameters will deviate from these depending on the treatment application and physician preference. One advantage of predetermined/pre-packaged program is that it can be readily activated by a program number. A simple version of a programmer, adapted to activate only a limited number of predetermined/pre-packaged programs may also be supplied to the patient.

In addition, each parameter may be individually adjusted and stored in the memory 394. The range of programmable electrical stimulation parameters include both stimulating and blocking frequencies, and are shown in table five below.

TABLE 5
Programmable electrical parameter range

| PARAMER | RANGE |
|---|---|
| Pulse Amplitude | 0.1 Volt-15 Volts |
| Pulse width | 20 μS-5 mSec. |
| Stim. Frequency | 5 Hz-200 Hz |
| Freq. for blocking | DC to 750 Hz |
| On-time | 5 Secs-24 hours |
| Off-time | 5 Secs-24 hours |
| Ramp | ON/OFF |

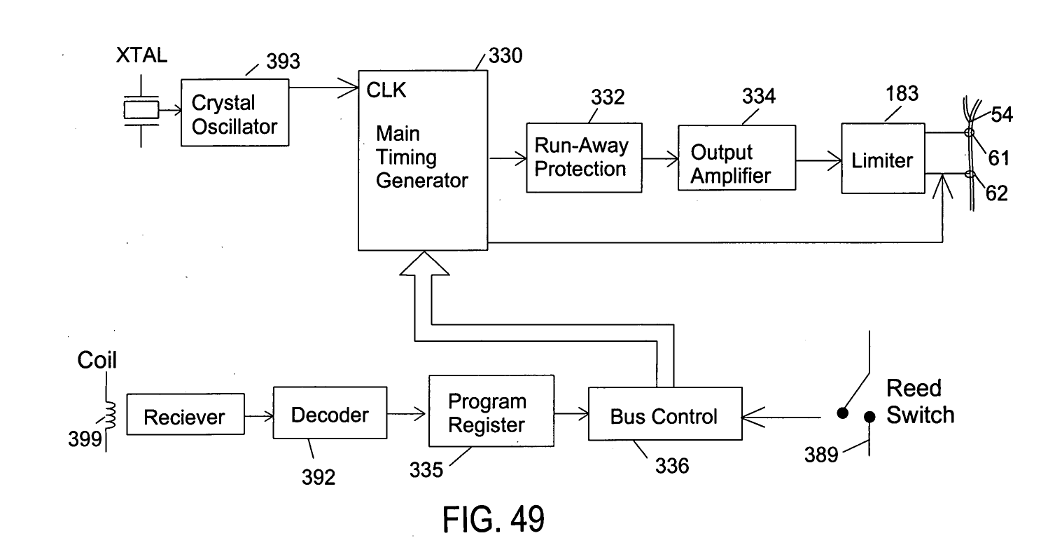

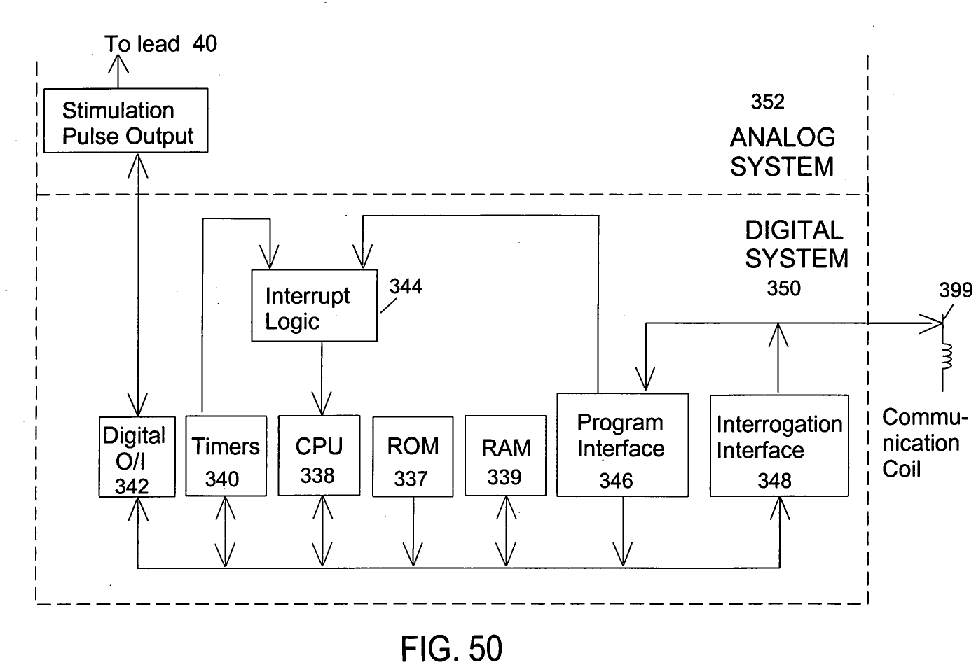

Shown in conjunction with FIGS. 49 and 50, the electronic stimulation module comprises both digital 350 and analog 352 circuits. A main timing generator 330 (shown in FIG. 39), controls the timing of the analog output circuitry for delivering neuromodulating pulses to the vagus nerve 54, via output amplifier 334. Limiter 183 prevents excessive stimulation energy from getting into the vagus nerve 54. The main timing generator 330 receiving clock pulses from crystal oscillator 393. Main timing generator 330 also receiving input from programmer 85 via coil 399. FIG. 36 highlights other portions of the digital system such as CPU 338, ROM 337, RAM 339, program interface 346, interrogation interface 348, timers 340, and digital O/I 342.

Most of the digital functional circuitry 350 is on a single chip (IC). This monolithic chip along with other IC's and components such as capacitors and the input protection diodes are assembled together on a hybrid circuit. As well known in the art, hybrid technology is used to establish the connections between the circuit and the other passive components. The integrated circuit is hermetically encapsulated in a chip carrier. A coil 399 situated under the hybrid substrate is used for bidirectional telemetry. The hybrid and battery 397 are encased in a titanium can 65. This housing is a two-part titanium capsule that is hermetically sealed by laser welding. Alternatively, electron-beam welding can also be used. The header 79 is a cast epoxy-resin with hermetically sealed feed-through, and form the lead 40 connection block.

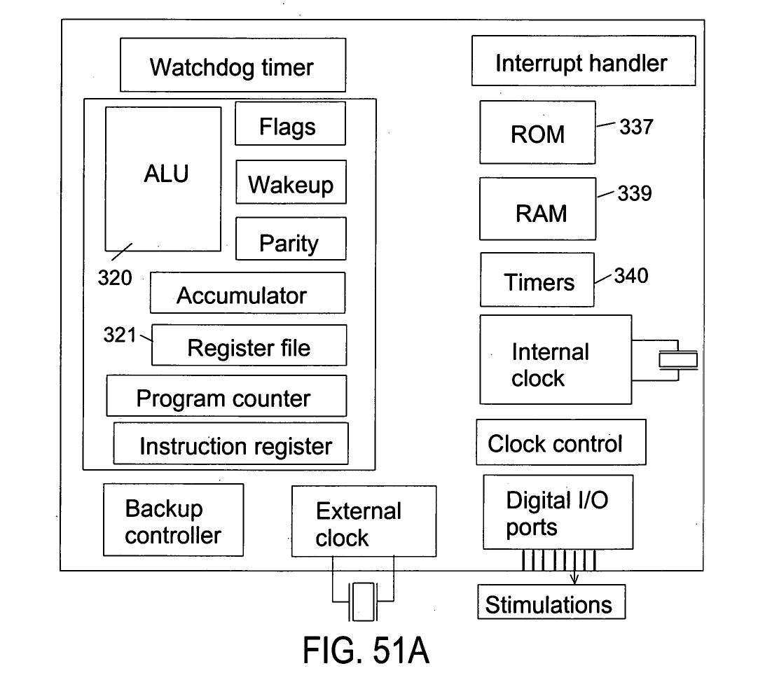

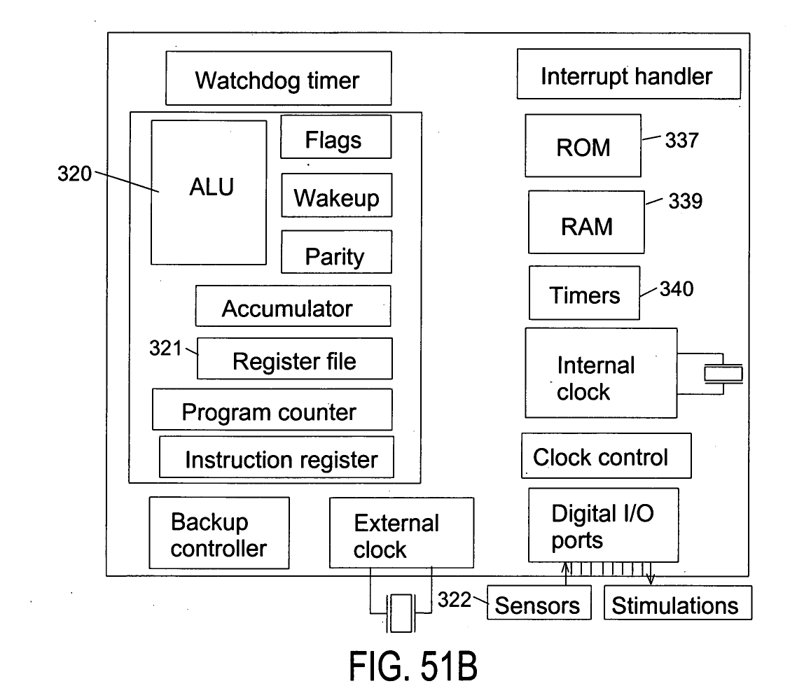

For further details, FIG. 51A highlights the general components of an 8-bit microprocessor as an example. It will be obvious to one skilled in the art that higher level microprocessor, such as a 16-bit or 32-bit may be utilized, and is considered within the scope of this invention. It comprises a ROM 337 to store the instructions of the program to be executed and various programmable parameters, a RAM 339 to store the various intermediate parameters, timers 340 to track the elapsed intervals, a register file 321 to hold intermediate values, an ALU 320 to perform the arithmetic calculation, and other auxiliary units that enhance the performance of a microprocessor-based IPG system.

The size of ROM 337 and RAM 339 units are selected based on the requirements of the algorithms and the parameters to be stored. The number of registers in the register file 321 are decided based upon the complexity of computation and the required number of intermediate values. Timers 340 of different precision are used to measure the elapsed intervals. Even though this embodiment does not have external sensors to control timing, future embodiments may have sensors 322 to effect the timing as shown in conjunction with FIG. 51B.

In this embodiment, the two main components of microprocessor are the datapath and control. The datapath performs the arithmetic operation and the control directs the datapath, memory, and I/O devices to execute the instruction of the program. The hardware components of the microprocessor are designed to execute a set of simple instructions. In general the complexity of the instruction set determines the complexity of datapth elements and controls of the microprocessor.

In this embodiment, the microprocessor is provided with a fixed operating routine. Future embodiments may be provided with the capability of actually introducing program changes in the implanted pulse generator. The instruction set of the microprocessor, the size of the register files, RAM and ROM are selected based on the performance needed and the type of the algorithms used. In this application of pulse generator, in which several algorithms can be loaded and modified, Reduced Instruction Set Computer (RISC) architecture is useful. RISC architecture offers advantages because it can be optimized to reduce the instruction cycle which in turn reduces the run time of the program and hence the current drain. The simple instruction set architecture of RISC and its simple hardware can be used to implement any algorithm without much difficulty. Since size is also a major consideration, an 8-bit microprocessor is used for the purpose. As most of the arithmetic calculation are based on a few parameters and are rather simple, an accumulator architecture is used to save bits from specifying registers. Each instruction is executed in multiple clock cycles, and the clock cycles are broadly classified into five stages: an instruction fetch, instruction decode, execution, memory reference, and write back stages. Depending on the type of the instruction, all or some of these stages are executed for proper completion.

Initially, an optimal instruction set architecture is selected based on the algorithm to be implemented and also taking into consideration the special needs of a microprocessor based implanted pulse generator (IPG). The instructions are broadly classified into Load/store instructions, Arithmetic and logic instructions (ALU), control instructions and special purpose instructions.

The instruction format is decided based upon the total number of instructions in the instruction set. The instructions fetched from memory are 8 bits long in this example. Each instruction has an opcode field (2 bits), a register specifier field (3-bits), and a 3-bit immediate field. The opcode field indicates the type of the instruction that was fetched. The register specifier indicates the address of the register in the register file on which the operations are performed. The immediate field is shifted and sign extended to obtain the address of the memory location in load/store instruction. Similarly, in branch and jump instruction, the offset field is used to calculate the address of the memory location the control needs to be transferred to.

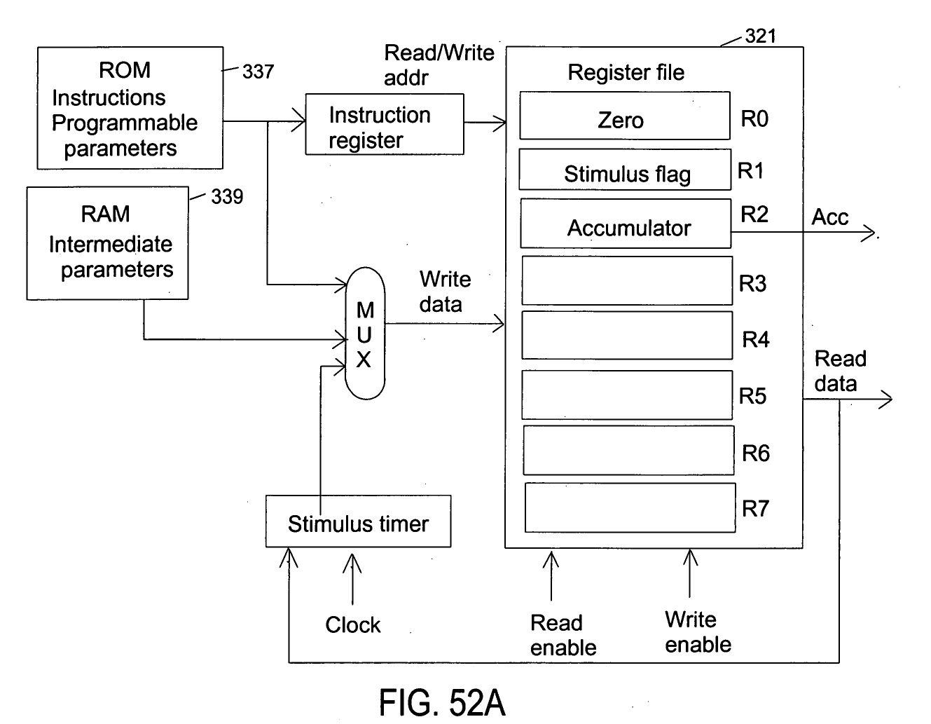

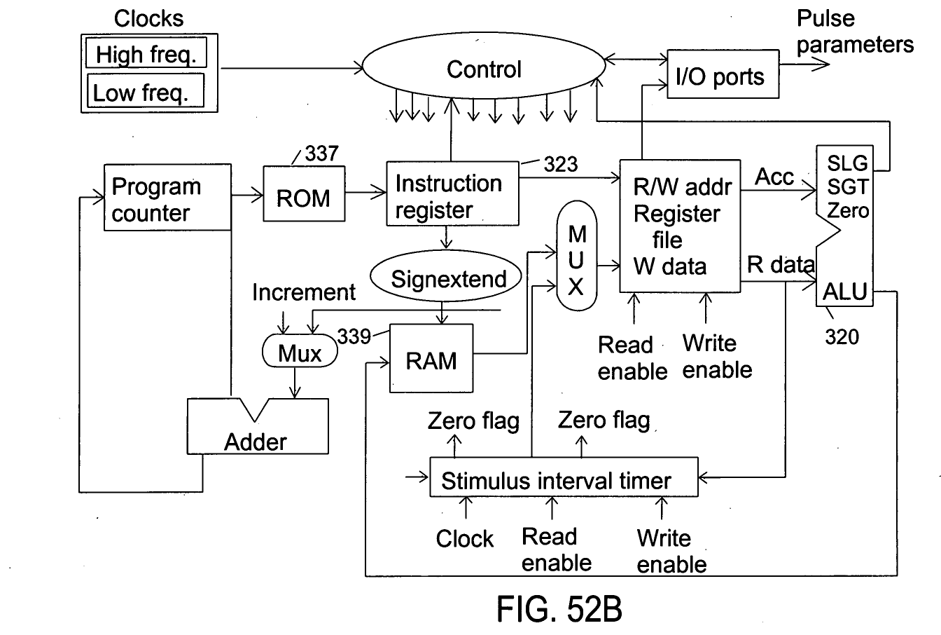

Shown in conjunction with FIG. 52A, the register file 321, which is a collection of registers in which any register can be read from or written to specifying the number of the register in the file. Based on the requirements of the design, the size of the register file is decided. For the purposes of implementation of stimulation pulses algorithms, a register file of eight registers is sufficient, with three special purpose register (0-2) and five general purpose registers (3-7), as shown in FIG. 52A: Register “0” always holds the value “zero”. Register “1” is dedicated to the pulse flags. Register “2” is an accumulator in which all the arithmetic calculations are performed. The read/write address port provides a 3-bit address to identify the register being read or written into. The write data port provides 8-bit data to be written into the registers either from ROM/RAM or timers. Read enable control, when asserted enables the register file to provide data at the read data port. Write enable control enables writing of data being provided at the write data port into a register specified by the read/write address.

Generally, two or more timers are required to implement the algorithm for the IPG. The timers are read and written into just as any other memory location. The timers are provided with read and write enable controls.

The arithmetic logic unit i s an important component of the microprocessor. It performs the arithmetic operation such as addition, subtraction and logical operations such as AND and OR. The instruction format of ALU instructions consists of an opcode field (2 bits), a function field (2 bits) to indicate the function that needs to be performed, and a register specifier (3 bits) or an immediate field (4 bits) to provide an operand.

The hardware components discussed above constitute the important components of a datapath. Shown in conjunction with FIG. 52B, there are some special purpose registers such a program counter (PC) to hold the address of the instruction being fetched from ROM 337 and instruction register (IR) 323, to hold the instruction that is fetched for further decoding and execution. The program counter is incremented in each instruction fetch stage to fetch sequential instruction from memory. In the case of a branch or jump instruction, the PC multiplexer allows to choose from the incremented PC value or the branch or jump address calculated. The opcode of the instruction fetched (IR) is provided to the control unit to generate the appropriate sequence of control signals, enabling data flow through the datapath. The register specification field of the instruction is given as read/write address to the register file, which provides data from the specified field on the read data port. One port of the ALU is always provided with the contents of the accumulator and the other with the read data port. This design is therefore referred to as accumulator-based architecture. The sign-extended offset is used for address calculation in branch and jump instructions. The timers are used to measure the elapsed interval and are enabled to count down on a low-frequency clock. The timers are read and written into, just as any other memory location (FIG. 52B).

In a multicycle implementation, each stage of instruction execution takes one clock cycle. Since the datapath takes multiple clock cycles per instruction, the control must specify the signals to be asserted in each stage and also the next step in the sequence. This can be easily implemented as a finite state machine.

A finite state machine consists of a set of states and directions on how to change states. The directions are defined by a next-state function, which maps the current state and the inputs to a new state. Each stage also indicates the control signals that need to be asserted. Every state in the finite state machine takes one clock cycle. Since the instruction fetch and decode stages are common to all the instruction, the initial two states are common to all the instruction. After the execution of the last step, the finite state machine returns to the fetch state.

A finite state machine can be implemented with a register that holds the current stage and a block of combinational logic such as a PLA. It determines the datapath signals that need to be asserted as well as the next state. A PLA is described as an array of AND gates followed by an array of OR gates. Since any function can be computed in two levels of logic, the two-level logic of PLA is used for generating control signals.

The occurrence of a wakeup event initiates a stored operating routine corresponding to the event. In the time interval between a completed operating routine and a next wake up event, the internal logic components of the processor are deactivated and no energy is being expended in performing an operating routine.

A further reduction in the average operating current is obtained by providing a plurality of counting rates to minimize the number of state changes during counting cycles. Thus intervals which do not require great precision, may be timed using relatively low counting rates, and intervals requiring relatively high precision, such as stimulating pulse width, may be timed using relatively high counting rates.

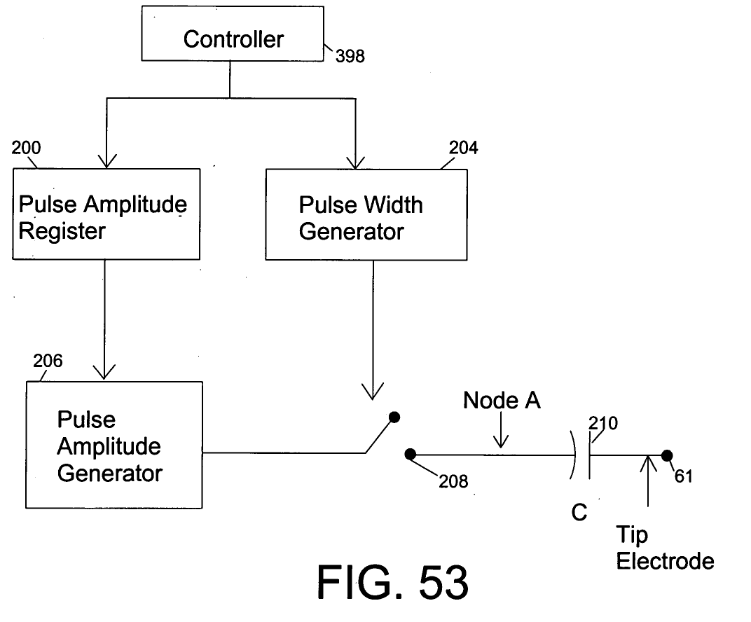

The logic and control unit 398 of the IPG controls the output amplifiers. The pulses have predetermined energy (pulse amplitude and pulse width) and are delivered at a time determined by the therapy stimulus controller. The circuitry in the output amplifier, shown in conjunction with (FIG. 53) generates an analog voltage or current that represents the pulse amplitude. The stimulation controller module initiates a stimulus pulse by closing a switch 208 that transmits the analog voltage or current pulse to the nerve tissue through the tip electrode 61 of the lead 40. The output circuit receiving instructions from the stimulus therapy controller 398 that regulates the timing of stimulus pulses and the amplitude and duration (pulse width) of the stimulus. The pulse amplitude generator 206 determines the configuration of charging and output capacitors necessary to generate the programmed stimulus amplitude. The output switch 208 is closed for a period of time that is controlled by the pulse width generator 204. When the output switch 208 is closed, a stimulus is delivered to the tip electrode 61 of the lead 40.

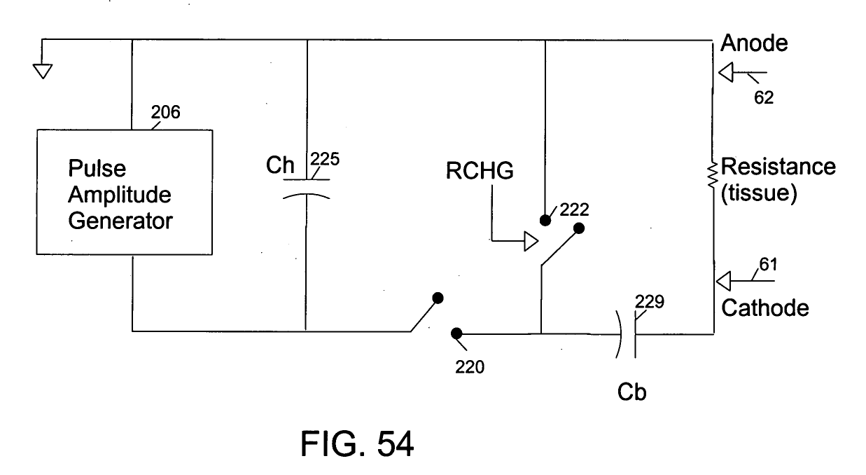

The constant-voltage output amplifier applies a voltage pulse to the distal electrode (cathode) 61 of the lead 40. A typical circuit diagram of a voltage output circuit is shown in FIG. 54. This configuration contains a stimulus amplitude generator 206 for generating an analog voltage. The analog voltage represents the stimulus amplitude and is stored on a holding capacitor Cₕ 225. Two switches are used to deliver the stimulus pulses to the lead 40, a stimulating delivery switch 220, and a recharge switch 222, that reestablishes the charge equilibrium after the stimulating pulse has been delivered to the nerve tissue. Since these switches have leakage currents that can cause direct current (DC) to flow into the lead system 40, a DC blocking capacitor C_{b} 229, is included. This is to prevent any possible corrosion that may result from the leakage of current in the lead 40. When the stimulus delivery switch 220 is closed, the pulse amplitude analog voltage stored in the (Cₕ 225) holding capacitor is transferred to the cathode electrode 61 of the lead 40 through the coupling capacitor, C_{b} 229. At the end of the stimulus pulse, the stimulus delivery switch 220 opens. The pulse duration being the interval from the closing of the switch 220 to its reopening. During the stimulus delivery, some of the charge stored on Cₕ 225 has been transferred to C_{b} 229, and some has been delivered to the lead system 40 to stimulate the nerve tissue.

To re-establish equilibrium, the recharge switch 222 is closed, and a rapid recharge pulse is delivered. This is intended to remove any residual charge remaining on the coupling capacitor C_{b} 229, and the stimulus electrodes on the lead (polarization). Thus, the stimulus is delivered as the result of closing and opening of the stimulus delivery 220 switch and the closing and opening of the RCHG switch 222. At this point, the charge on the holding Cₕ 225 must be replenished by the stimulus amplitude generator 206 before another stimulus pulse can be delivered.

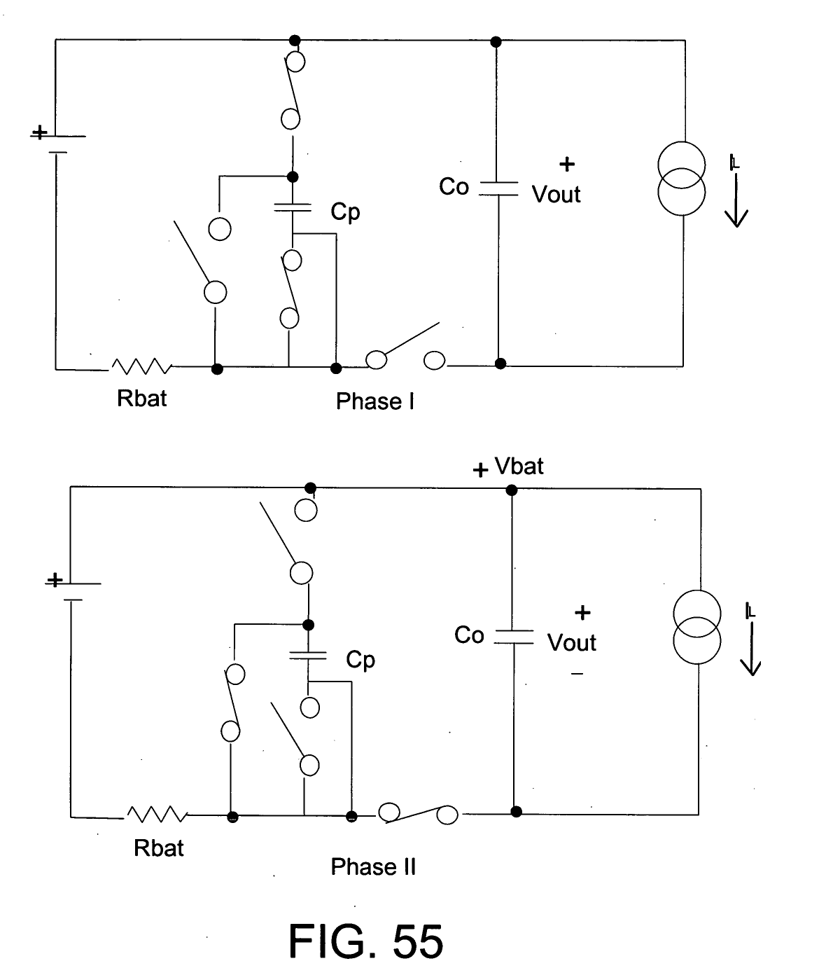

The pulse generating unit charges up a capacitor and the capacitor is discharged when the control (timing) circuitry requires the delivery of a pulse. This embodiment utilizes a constant voltage pulse generator, even though a constant current pulse generator can also be utilized. Pump-up capacitors are used to deliver pulses of larger magnitude than the potential of the batteries. The pump up capacitors are charged in parallel and discharged into the output capacitor in series. Shown in conjunction with FIG. 55 is a circuit diagram of a voltage doubler which is shown here as an example. For higher multiples of battery voltage, this doubling circuit can be cascaded with other doubling circuits. As shown in FIG. 55, during phase I (top of FIG. 55), the pump capacitor Cₚ is charged to V_{bat} and the output capacitor Cₒ supplies charge to the load. During phase II, the pump capacitor charges the output capacitor, which is still supplying the load current. In this case, the voltage drop across the output capacitor is twice the battery voltage.

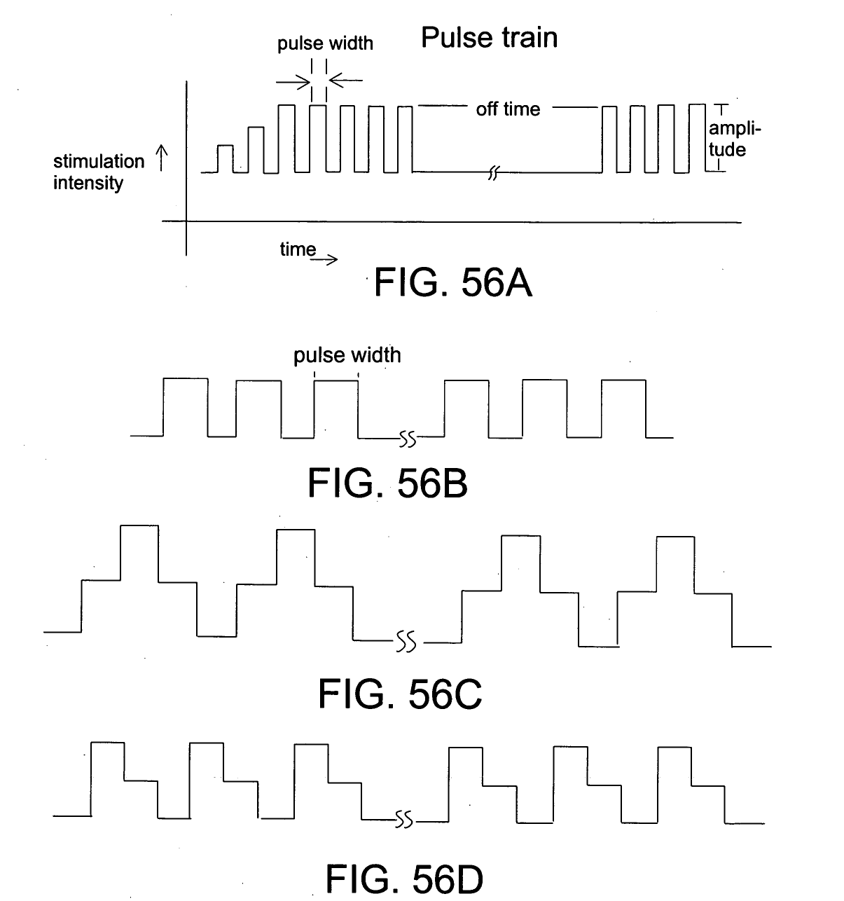

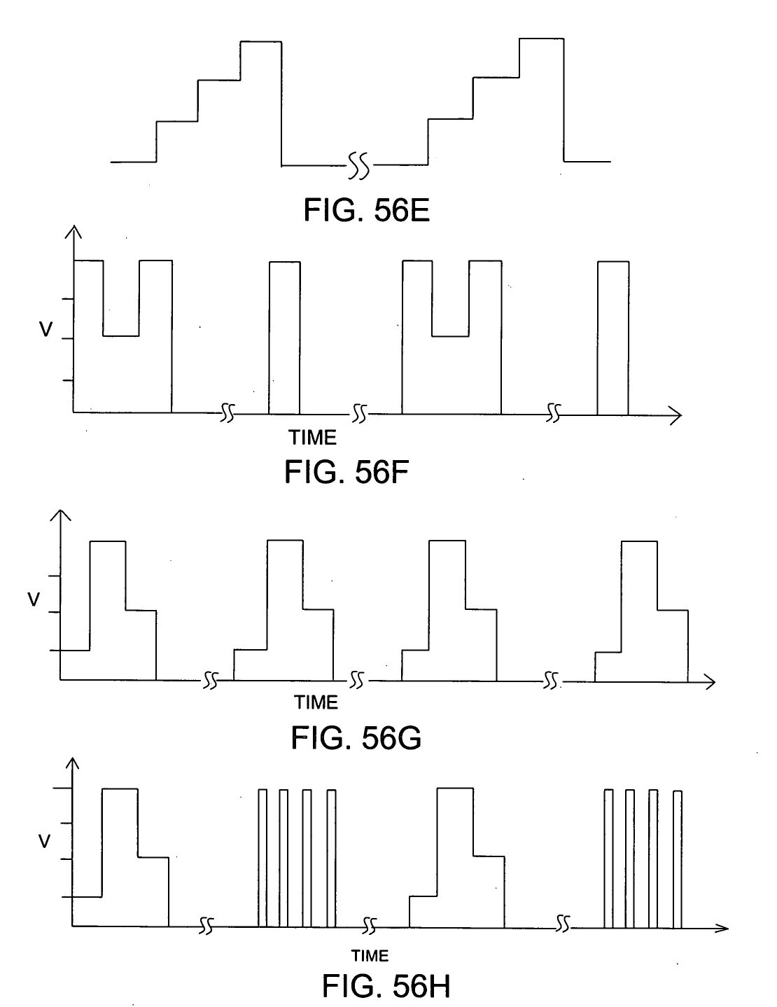

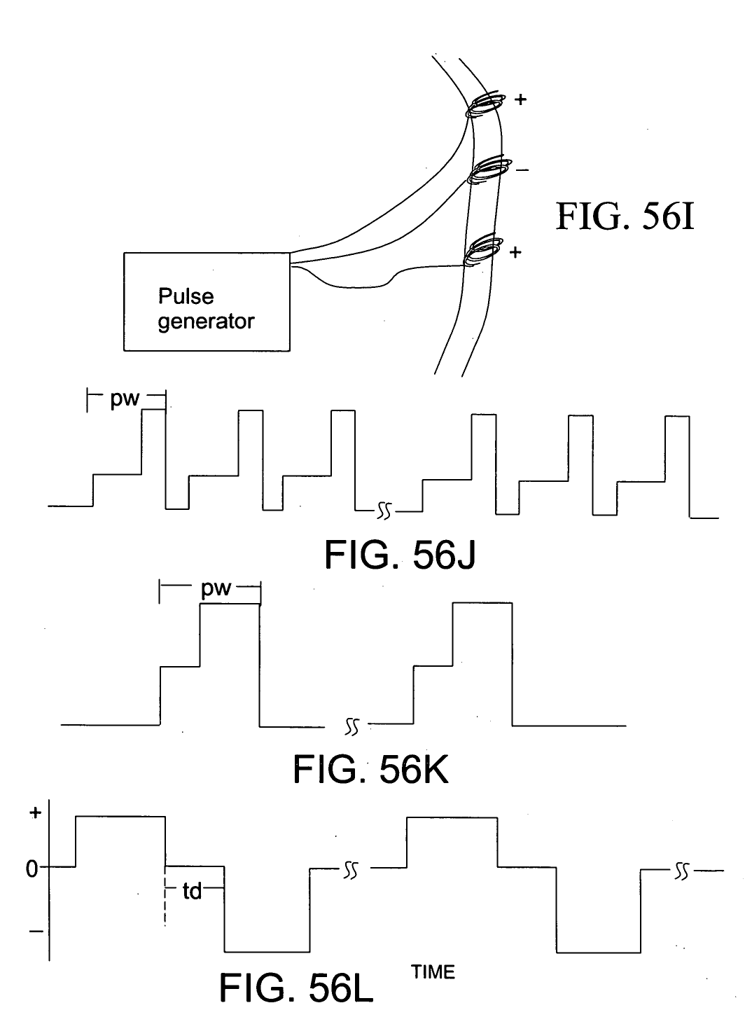

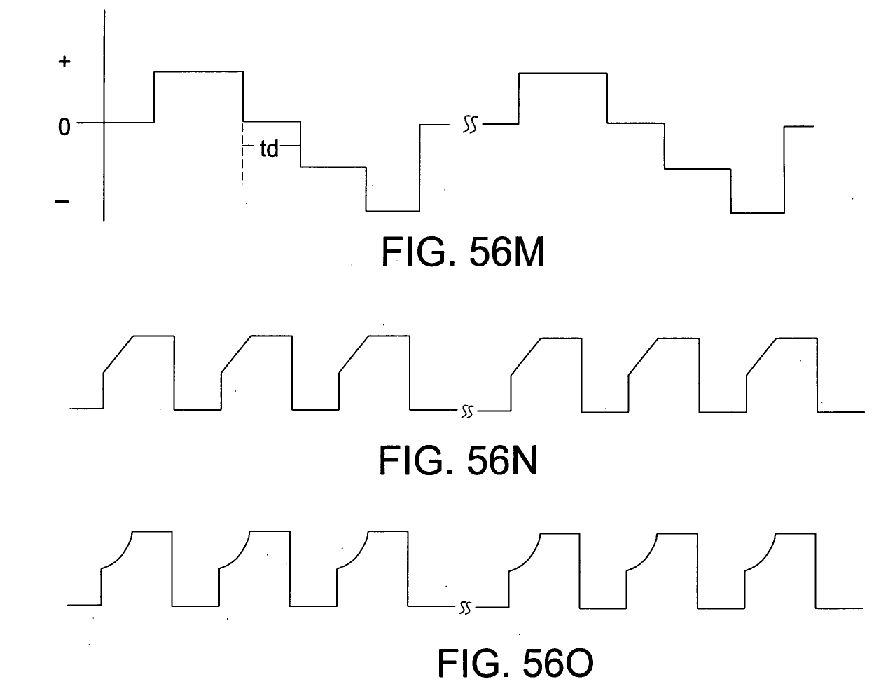

FIG. 56A shows one example of the pulse trains that may be delivered with this embodiment or in prior art vagus nerve stimulators. The microcontroller is configured to deliver the pulse train as shown in the figure, i.e. there is “ramping up” of the pulse train. The purpose of the ramping-up is to avoid sudden changes in stimulation, when the pulse train begins. The ramping-up or ramping-down is optional, and may be programmed into the microcontroller.

The prior art systems delivering fixed rectangular pulses provide limited capability for selective stimulation or neuromodulation of vagus nerve(s). A fixed rectangular pulse, whether constant voltage or constant current, will recruit either i) A-fibers, or ii) A and B fibers, or iii) A and B and C fibers. Only one of these three discrete states can be achieved. This form of modulation is severely limited for providing therapy for neurological disorders.

In the method and system of the current invention, the microcontroller is configured to deliver rectangular and complex pulses. Complex pulses comprise non-rectangular, biphasic, multi-step, and other complex pulses where the amplitude is varying during the pulse. Advantageously, these complex pulses provide a new dimension to selective stimulation or neuromodulation of vagus nerve(s) to provide therapy for neurological disorders, such as involuntary movement disorders.

Examples of these pulses and pulse trains are shown in FIGS. 56B to 56H. Selective stimulation with these complex pulses takes into account the threshold properties of different types of nerve fibers, as well as, the different refractory properties of different types of nerve fibers that are contained in the vagus nerve(s).

For example in the multi-step pulse shown in FIG. 56C, the first part of the pulse will tend to recruit large diameter (and myelinated) fibers, such as A and B fibers. The middle portion of the pulse where the amplitude is highest, will tend to recruit c-fibers which are the smallest fibers, and the last portion of the pulse will again tend to recruit the large diameter fibers provided they are not refractory. The multi-step (and multi-amplitude) pulses shown in FIG. 56E will tend to recruit large diameter fibers initially, and the later part of the pulse will tend to recruit the smaller diameter C-fibers.

Further, as shown in the examples of FIGS. 56F and 56H, complex and simple pulses, or pulse trains may be alternated. It will be clear to one skilled in the art, that the pulse trains in these two examples take into account both the threshold properties and the refractory properties of different types of nerve fibers which were shown in FIG. 9.

The pulses and pulse trains of this disclosure gives physicians a lot of flexibility for trying various different neuromodulation algorithms, for providing and optimizing therapy for involuntary movement disorders.

Furthermore, as shown in conjunction with FIG. 56-I, a combination of tripolar electrodes with different pulse shapes may be used for selective stimulation of vagus nerve(s).

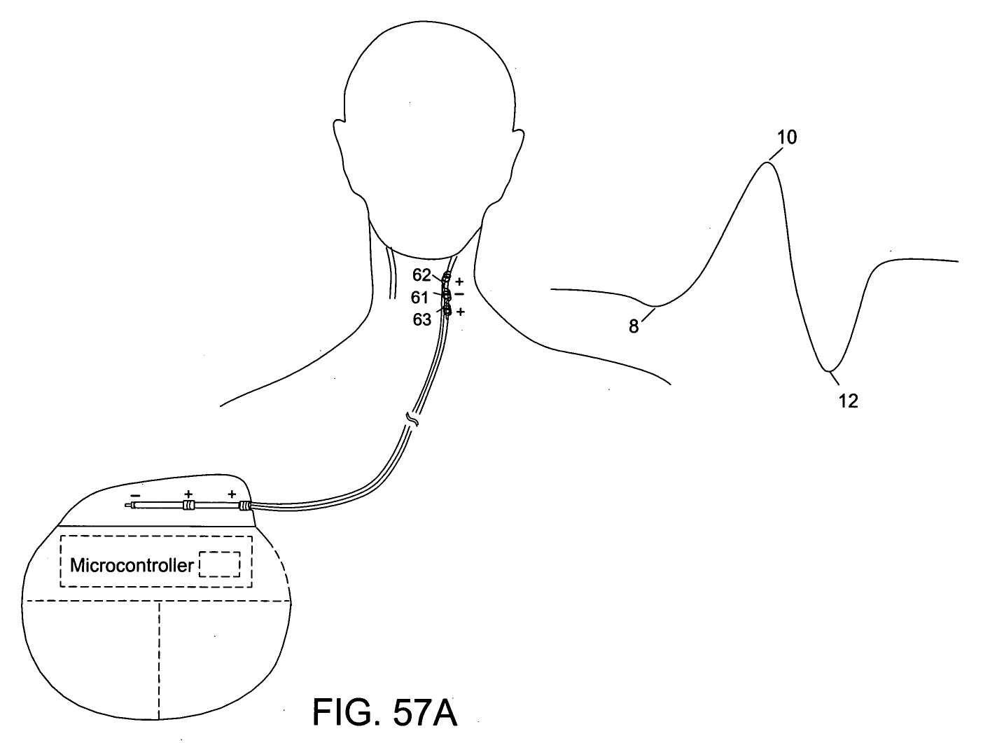

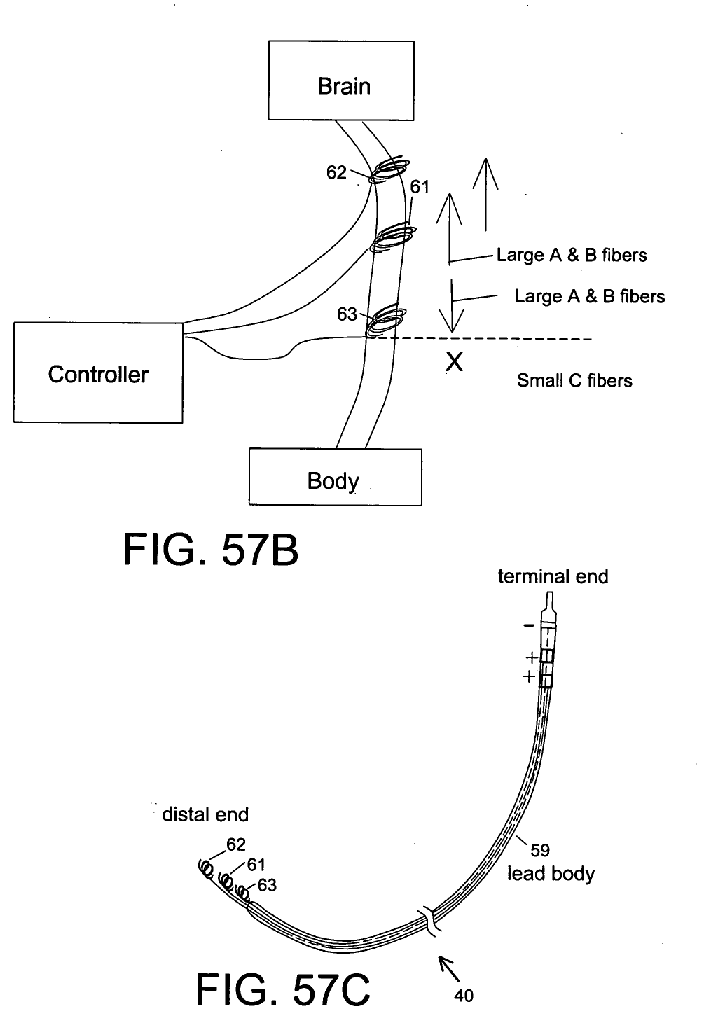

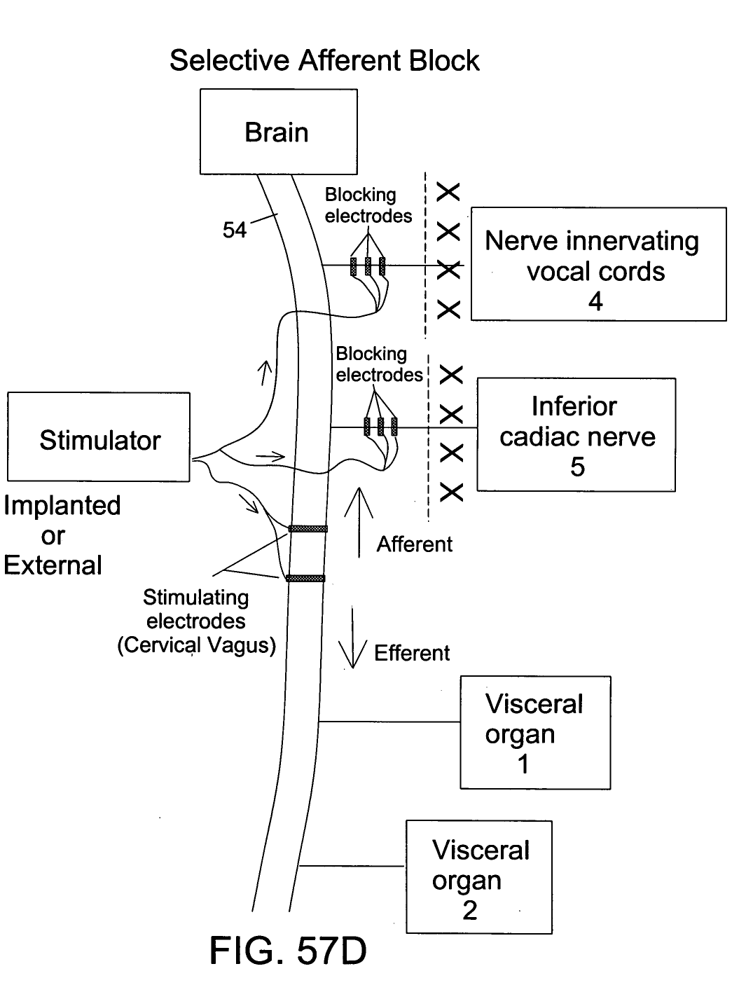

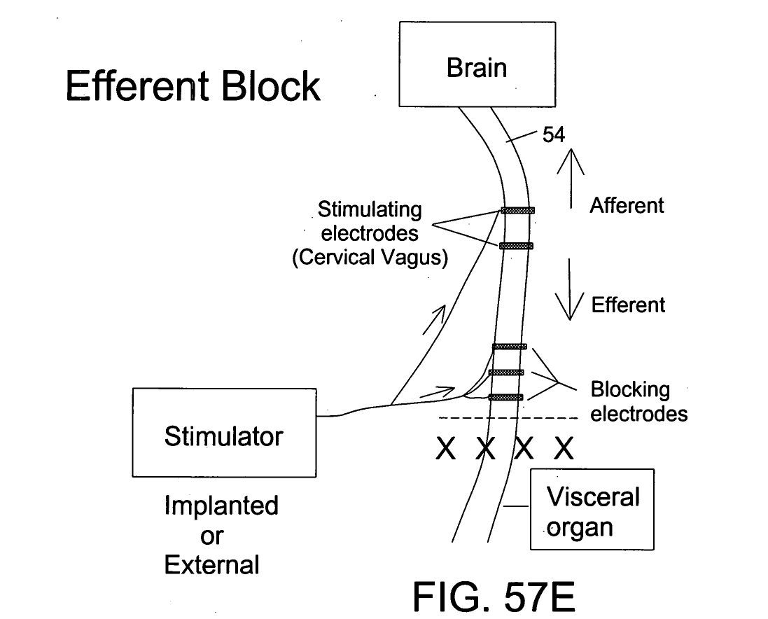

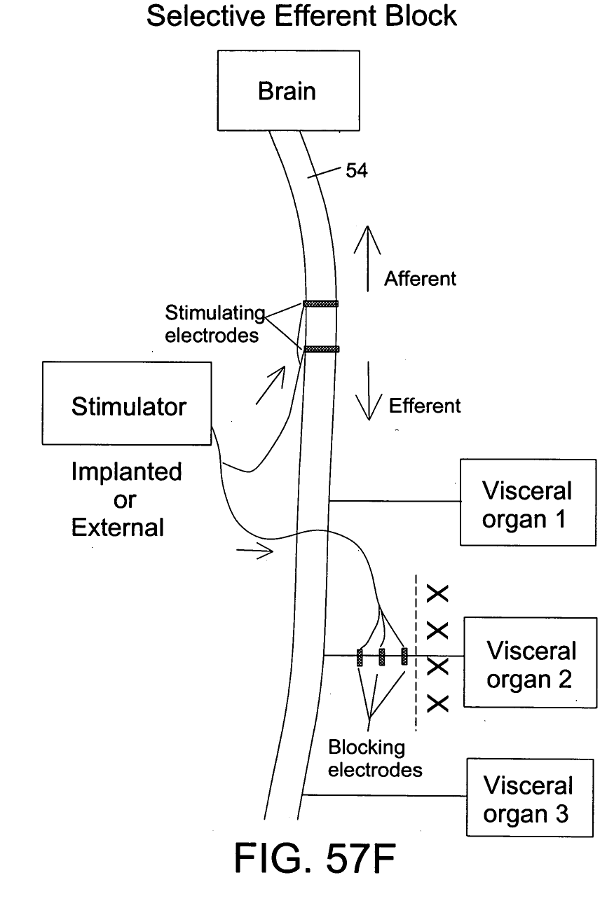

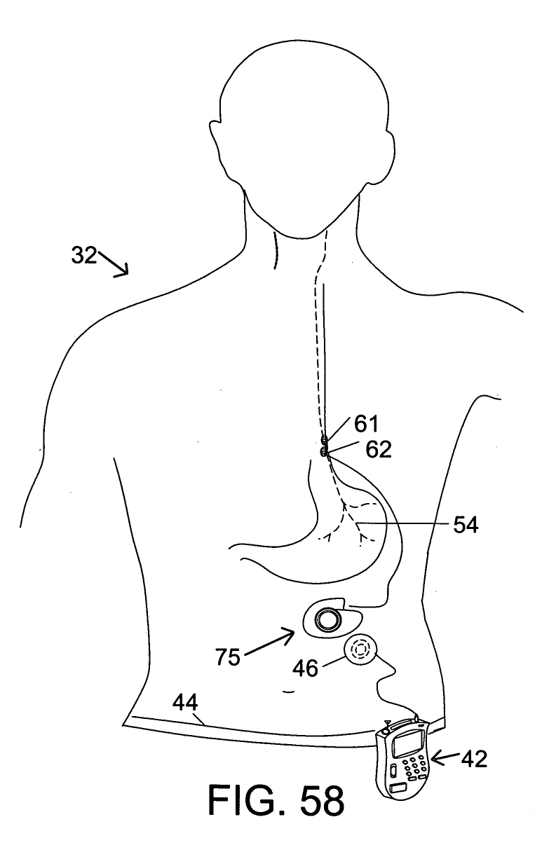

The different pulses used in conjunction with tripolar electrodes are shown in conjunction with FIGS. 56J, 56K, 57L, 56M, 56N, and 56-O. This combination is advantageous, because it can be used to provide selective large fiber block as well. As was previously pointed out in Table 2, the vagus nerve also comprises motor components which innervate the soft palate, pharynx, larynx, and upper esophagus. One of the clinical side effects of vagus nerve stimulation is hoarsness of the throat and voice change.

The combination of tripolar electrodes and the pulse shapes of FIGS. 56-J to 56-O would not only decrease or prevent the unwanted side effects, but the electrical charge of the pulse is also reduced, which will make this technique safer for long-term clinical applications.

In the tripolar cuff electrodes (FIG. 56-I), the electrode consists of a cathode, flanked by two anodes. When stimulation is applied, the nerve membrane is depolarized near the cathode and hyperpolarized near the anodes. If the membrane is sufficiently hyperpolarized, an action potential (AP) that travels into the depolarized zone cannot pass the hyperpolarized zone and is arrested. As with excitation, a lower external stimulus is needed for blocking large diameter fibers than for blocking smaller ones (C-fibers). Therefore, by applying a current above the blocking threshold for the large fibers but below the blocking threshold for the smaller ones, selective activation of the small fibers can be obtained. This is one of the aims of this invention, where selective stimulation of C-fibers can be achieved, without the unwanted side effects of motor stimulation to the throat region.

As shown in FIGS. 56J and 56K, the microcontroller 398 in the pulse generator 391 is configured to provide stepped pulses. The current of the first step is too low to induce an action potential (AP), but only depolarizes the membrane. The AP is generated during the second step. The pulses in FIG. 56J and 56K are similar, except that the pulses in FIG. 56J have a longer first step. In addition to anodel blocking, another advantage of these stepped pulses is that the total charge per pulse can be reduced by almost a third.

Other examples of complex pulses, that may be used with tripolar electrodes are shown in FIGS. 56-L to 56-O. FIG. 56L shows biphasic pulses with a time delay t_{d} between the positive and negative pulse. FIG. 56M shows biphasic pulses with a time delay t_{d}, where the second part of the pulse is a step pulse. FIG. 56N shows ramp pulses, and FIG. 56-O show pulses with exponential components. Theoretical work, computer modeling, and animal studies have all shown that lower charge is obtained with these modified pulses when compared to square pulses. The charge reduction of these pulses can be approximately 30% less when compared to square pulses, which is fairly significant. The microcontroller 398 of the pulse generator 391 can be configured to deliver these pulses, as is well known to one skilled in the art.

Since the number of types of pulses and pulse trains to provide therapy can be complex for many physician's, in one aspect pre-determined/pre-packaged program comprise a complete program for the pulse trains that deliver therapy. The advantage of the pre-packaged programs is that the physician may program a complicated program simply by selecting a program number.

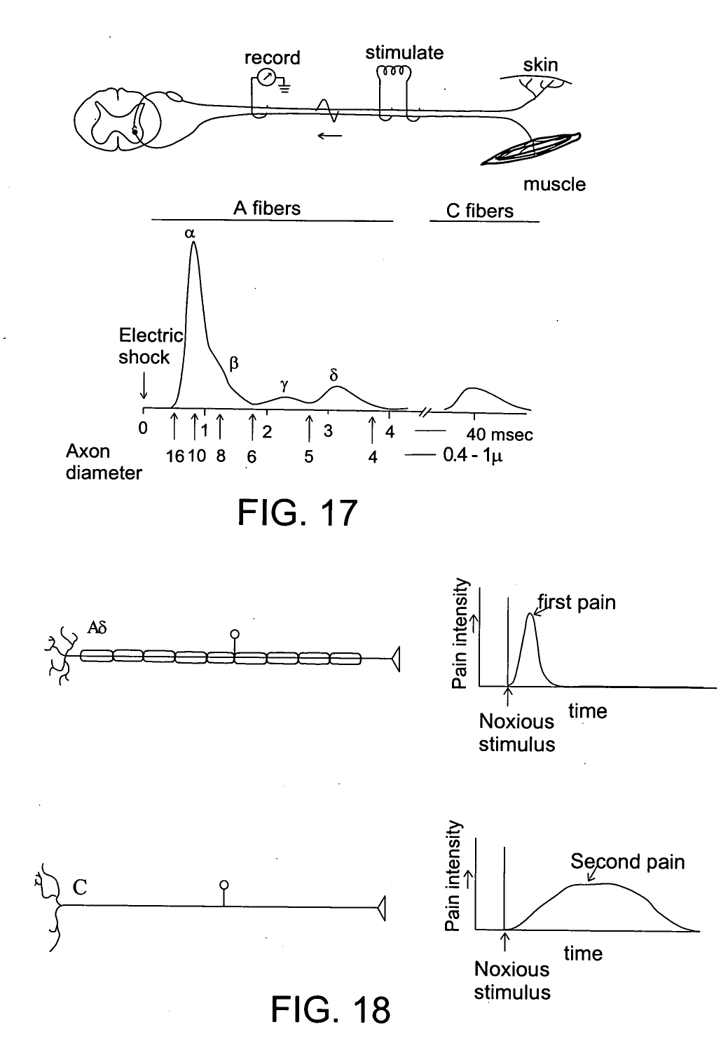

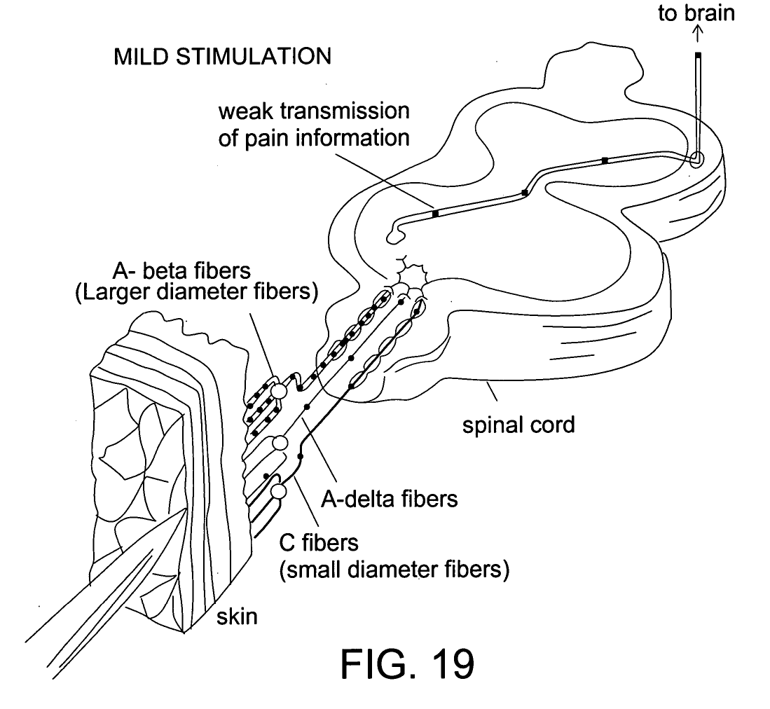

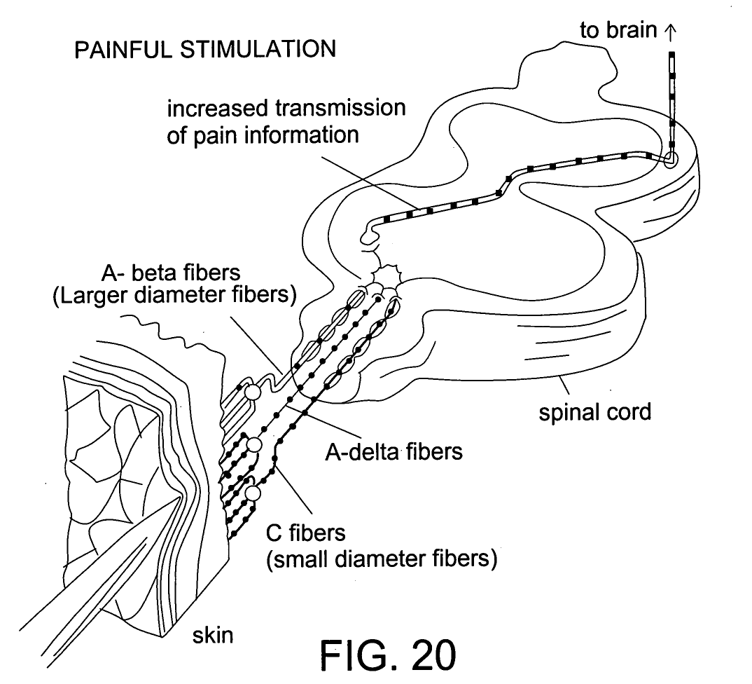

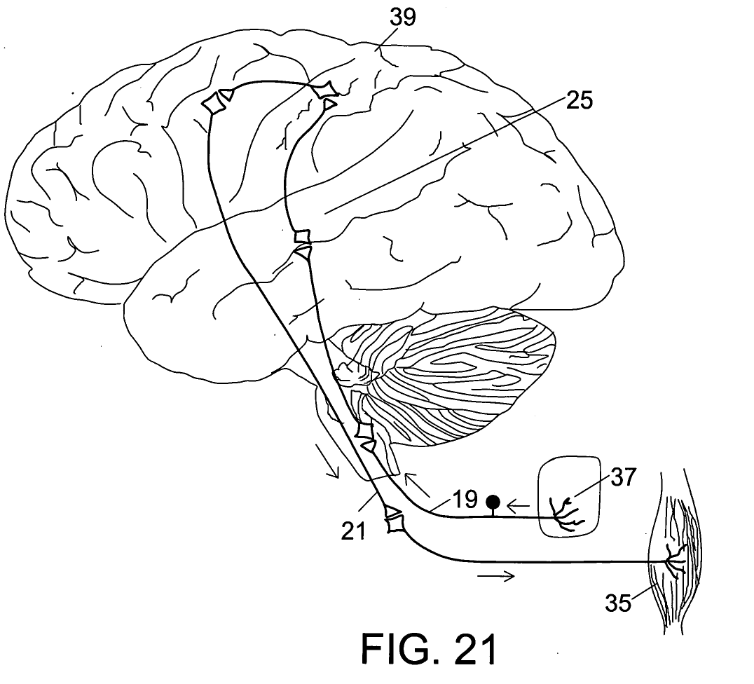

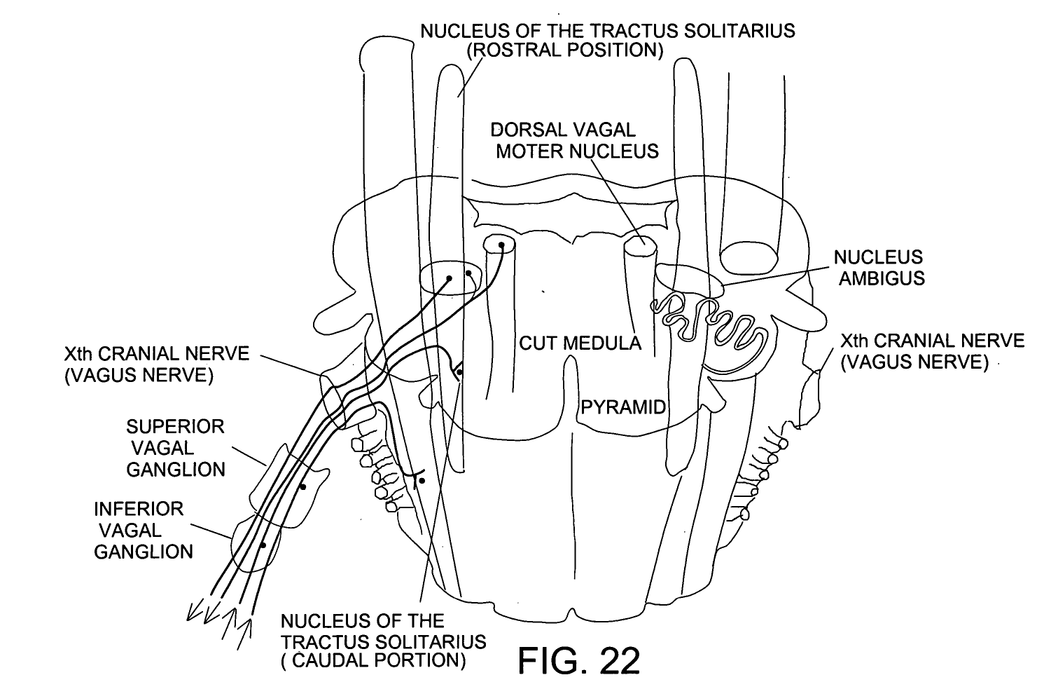

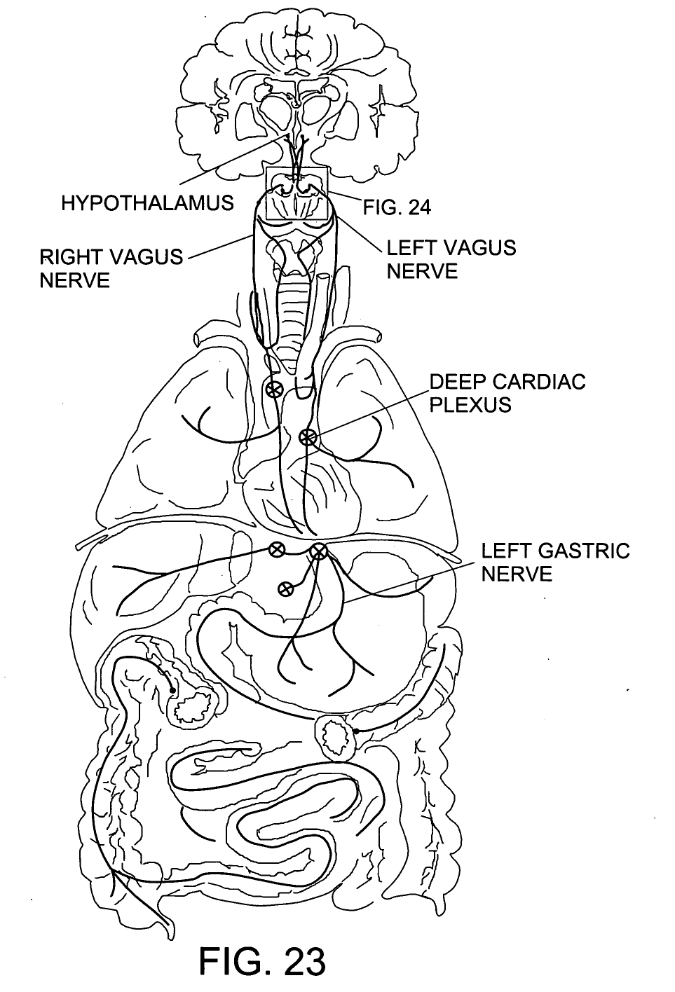

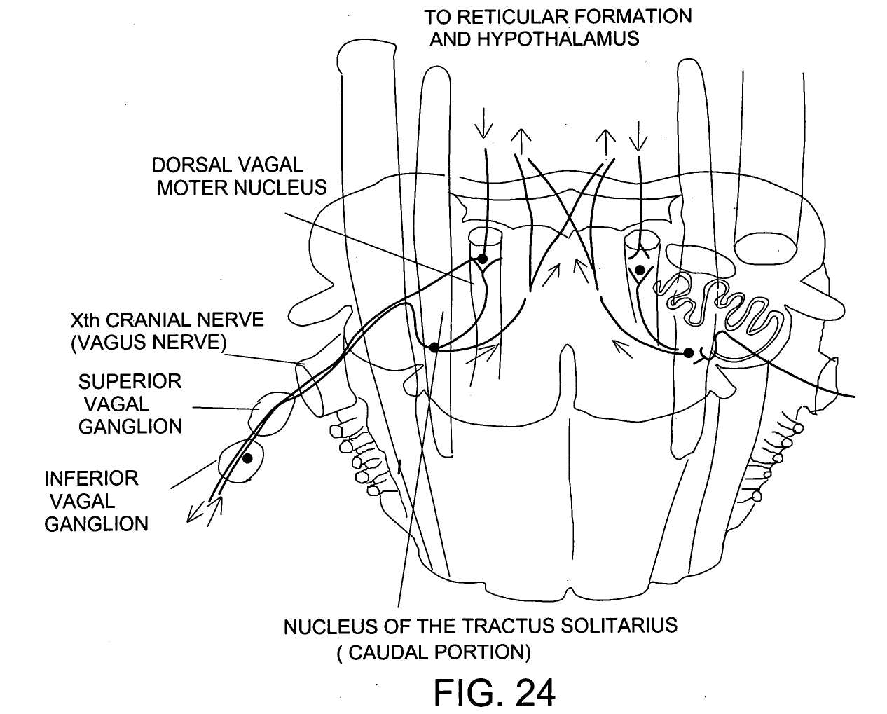

Since a key concept of this invention is to deliver afferent stimulation, in one aspect efferent stimulation of selected types of fibers may be substantially blocked, utilizing the “greenwave” effect. In such a case, as shown in conjunction with FIGS. 57A and 57B, a tripolar lead is utilized. As depicted on the top right portion of FIG. 57A, a depolarization peak 10 on the vagus nerve bundle corresponding to electrode 61 (cathode) and the two hyper-polarization peaks 8, 12 corresponding to electrodes 62, 63 (anodes). With the microcontroller controlling the tripolar device, the size and timing of the hyper-polarizations 8,12 can be controlled. As was shown previously in FIGS. 9 and 17, since the speed of conduction is different between the larger diameter A and B fibers and the smaller diameter c-fibers, by appropriately timing the pulses, collision blocks can be created for conduction via the large diameter A and B fibers in the efferent direction. This is depicted schematically in FIG. 57B. A lead with tripolar electrodes for stimulation/blocking is shown in conjunction with FIG. 57C. Alternatively, separate leads may be utilized for stimulation and blocking, and the pulse generator may be adapted for two or three leads, as is well known in the art for dual chamber cardiac pacemakers or implantable defibrillators.

Therefore in the method and system of this invention, stimulation without block may be provided. Additionally, stimulation with selective block may be provided. Blocking of nerve impulses, unidirectional blocking, and selective blocking of nerve impulses is well known in the scientific literature. Some of the general literature is listed below and is incorporated herein by reference. (a) “Generation of unidirectionally propagating action potentials using a monopolar electrode cuff”, Annals of Biomedical Engineering, volume 14, pp. 437-450, By Ira J. Ungar et al. (b) “An asymmetric two electrode cuff for generation of unidirectionally propagated action potentials”, IEEE Transactions on Biomedical Engineering, volume BME-33, No. 6, June 1986, By James D. Sweeney, et al. (c) A spiral nerve cuff electrode for peripheral nerve stimulation, IEEE Transactions on Biomedical Engineering, volume 35, No. 11, November 1988, By Gregory G. Naples. et al. (d) “A nerve cuff technique for selective excitation of peripheral nerve trunk regions, IEEE Transactions on Biomedical Engineering, volume 37, No. 7, July 1990, By James D. Sweeney, et al. (e) “Generation of unidirectionally propagated action potentials in a peripheral nerve by brief stimuli”, Science, volume 206 pp. 1311-1312, Dec. 14, 1979, By Van Den Honert et al. (f) A technique for collision block of perpheral nerve: Frequency dependence” IEEE Transactions on Biomedical Engineering, MP-12, volume 28, pp. 379-382, 1981, By Van Den Honert et al. (g) “A nerve cuff design for the selective activation and blocking of myelinated nerve fibers” Ann. Conf. of the IEEE Engineering in Medicine and Biology Soc., volume 13, No. 2, p 906, 1991, By D. M Fitzpatrick et al. (h) “Orderly recruitment of motoneurons in an acute rabbit model”, “Ann. Conf of the IEEE Engineering in Medicine and Biology Soc., volume 20, No. 5, page 2564, 1998, By N. J. M. Rijkhof, et al. (i) “Orderly stimulation of skeletal muscle motor units with tripolar nerve cuff electrode”, IEEE Transactions on Biomedical Engineering, volume 36, No. 8, pp. 836, 1989, By R. Bratta. (j) M. Devor, “Pain Networks”, Handbook of Brand Theory and Neural Networks, Ed. M. A. Arbib, MIT Press, page 698, 1998.

Blocking can be generally divided into 3 categories: (a) DC or anodal block, (b) Wedenski Block, and (c) Collision block. In anodal block there is a steady potential which is applied to the nerve causing a reversible and selective block. In Wedenski Block the nerve is stimulated at a high rate causing the rapid depletion of the neurotransmitter. In collision blocking, unidirectional action potentials are generated anti-dromically. The maximal frequency for complete block is the reciprocal of the refractory period plus the transit time, i.e. typically less than a few hundred hertz. The use of any of these blocking techniques can be applied for the practice of this invention, and all are considered within the scope of this invention.

Since one of the objects of this invention is to decease side effects such as hoarsness in the throat, or any cardiac side effects, blocking electrodes may be strategically placed at the relevant branches of vagus nerve.

As shown in conjunction with FIG. 57D, the stimulating electrodes are placed on cervical vagus, and the blocking electrodes are placed on a branch to vocal cords 4. With the blocking electrodes positioned between the vocal cords and the stimulating electrodes, and the controller supplying blocking pulses to the blocking electrode, the side effects pertaining to vocal response can be eliminated or significantly diminished. Advantageously, more aggressive therapy can be provided, leading to even better efficacy. Similarly, as also depicted in FIG. 57D, the blocking electrode may be placed on the inferior cardiac nerve 5, whereby the blocking electrode would be positioned between the heart and stimulating electrode. Again, with the controller delivering blocking pulses to the blocking electrode, the cardiac side effects would be significantly diminished or virtually eliminated.

Shown in conjunction with FIG. 57E is simplified depiction of efferent block. This time with the blocking electrode placed distal to the stimulating electrode, and the controller supplying blocking pulses to the blocking electrodes, the efferent pulses can be blocked. Advantageously, the side effects related to cardiopulmonary system, gastrointestinal system and pancreobiliary system can be greatly diminished. It will be apparent to one skilled in the art that, as shown in conjunction with 57F, selective efferent block can also be performed.

In one aspect of the invention, the pulsed electrical stimulation to the vagus nerve(s) may be provided anywhere along the length of the vagus nerve(s). As was shown earlier in conjunction with FIG. 30, the pulsed electrical stimulation may be at the cervical level. Alternatively, shown in conjunction with FIG. 48, the stimulation to the vagus nerve(s) may be around the diaphramatic level. Either above the diaphragm or below the diaphragm.

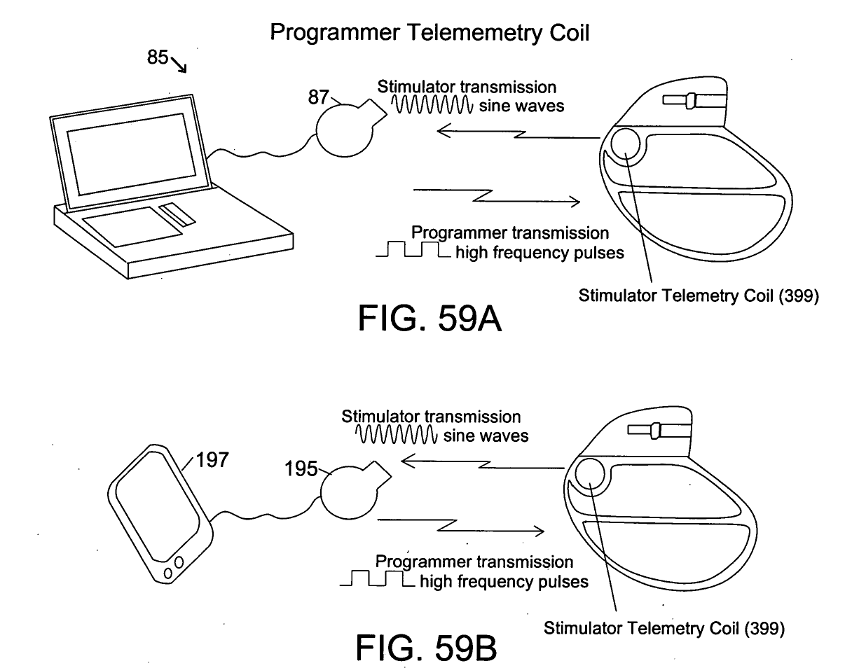

The programming of the implanted pulse generator (IPG) 391 is shown in conjunction with FIGS. 59A and 59B. With the magnetic Reed Switch 389 (FIG. 48) in the closed position, a coil in the head of the programmer 85, communicates with a telemetry coil. 399 of the implanted pulse generator 391. Bi-directional inductive telemetry is used to exchange data with the implanted unit 391 by means of the external programming unit 85.

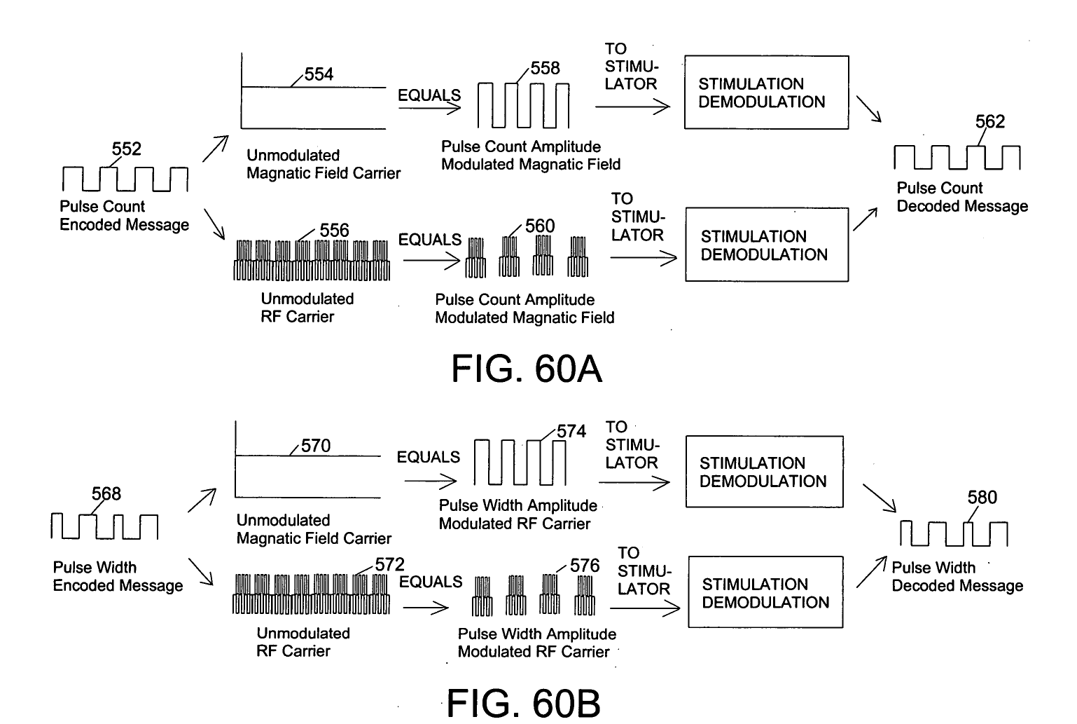

The transmission of programming information involves manipulation of the carrier signal in a manner that is recognizable by the pulse generator 391 as a valid set of instructions. The process of modulation serves as a means of encoding the programming instruction in a language that is interpretable by the implanted pulse generator 391. Modulation of signal amplitude, pulse width, and time between pulses are all used in the programming system, as will be appreciated by those skilled in the art. FIG. 60A shows an example of pulse count modulation, and FIG. 60B shows an example of pulse width modulation, that can be used for encoding.

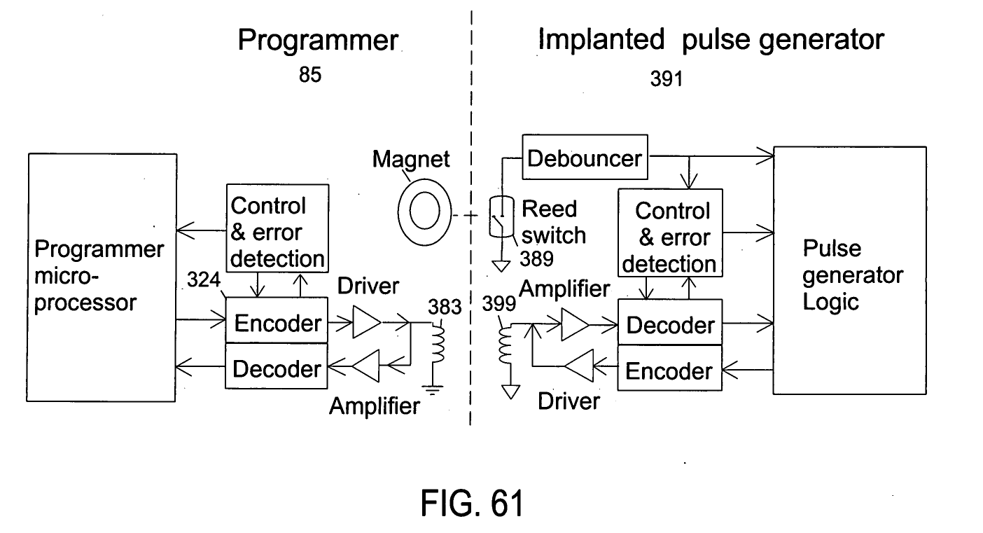

FIG. 61 shows a simplified overall block diagram of the implanted pulse generator (IPG) 391 programming and telemetry interface. The left half of FIG. 61 is programmer 85 which communicates programming and telemetry information with the IPG 391. The sections of the IPG 391 associated with programming and telemetry are shown on the right half of FIG. 61. In this case, the programming sequence is initiated by bringing a permanent magnet in the proximity of the IPG 391 which closes a reed switch 389 in the IPG 391. Information is then encoded into a special error-correcting pulse sequence and transmitted electromagnetically through a set of coils. The received message is decoded, checked for errors, and passed on to the unit's logic circuitry. The IPG 391 of this embodiment includes the capability of bidirectional communication.

The reed switch 389 is a magnetically-sensitive mechanical switch, which consists of two thin strips of metal (the “reed”) which are ferromagnetic. The reeds normally spring apart when no magnetic field is present. When a field is applied, the reeds come together to form a closed circuit because doing so creates a path of least reluctance. The programming head of the programmer contains a high-field-strength ceramic magnet.

When the switch closes, it activates the programming hardware, and initiates an interrupt of the IPG central processor. Closing the reed switch 389 also presents the logic used to encode and decode programming and telemetry signals. A nonmaskable interrupt (NMI) is sent to the IPG processor, which then executes special programming software. Since the NMI is an edge-triggered signal and the reed switch is vulnerable to mechanical bounce, a debouncing circuit is used to avoid multiple interrupts. The overall current consumption of the IPG increases during programming because of the debouncing circuit and other communication circuits.

A coil 399 is used as an antenna for both reception and transmission. Another set of coils 383 is placed in the programming head, a relatively small sized unit connected to the programmer 85. All coils are tuned to the same resonant frequency. The interface is half-duplex with one unit transmitting at a time.

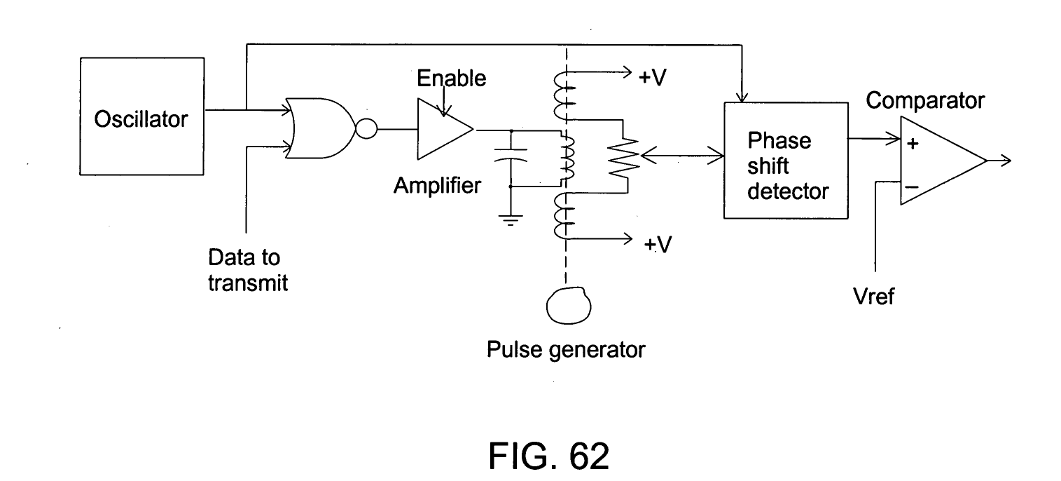

Since the relative positions of the programming head 87 and IPG 391 determine the coupling of the coils, this embodiment utilizes a special circuit which has been devised to aid the positioning of the programming head, and is shown in FIG. 62. It operates on similar principles to the linear variable differential transformer. An oscillator tuned to the resonant frequency of the pacemaker coil 399 drives the center coil of a three-coil set in the programmer head. The phase difference between the original oscillator signal and the resulting signal from the two outer coils is measured using a phase shift detector. It is proportional to the distance between the implanted pulse generator and the programmer head. The phase shift, as a voltage, is compared to a reference voltage and is then used to control an indicator such as an LED. An enable signal allows switching the circuit on and off.

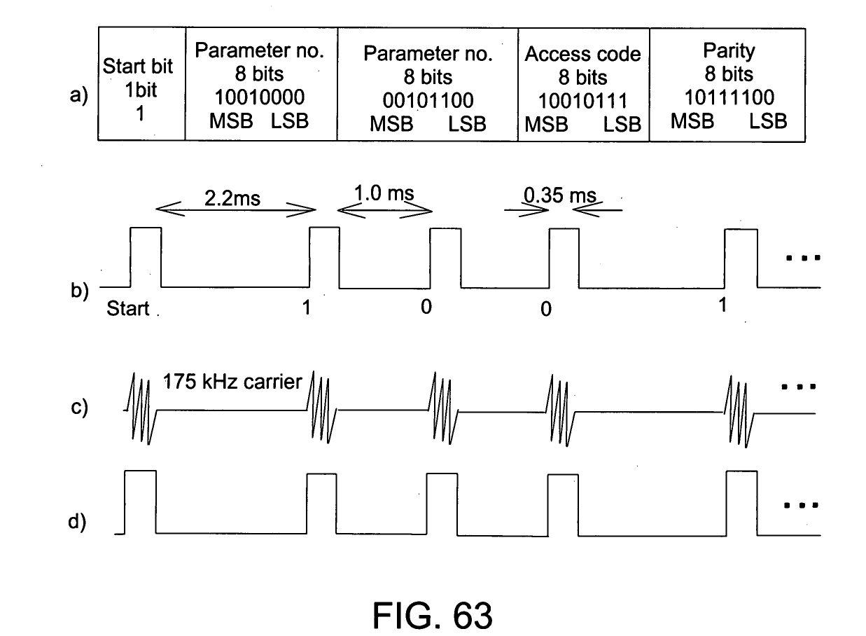

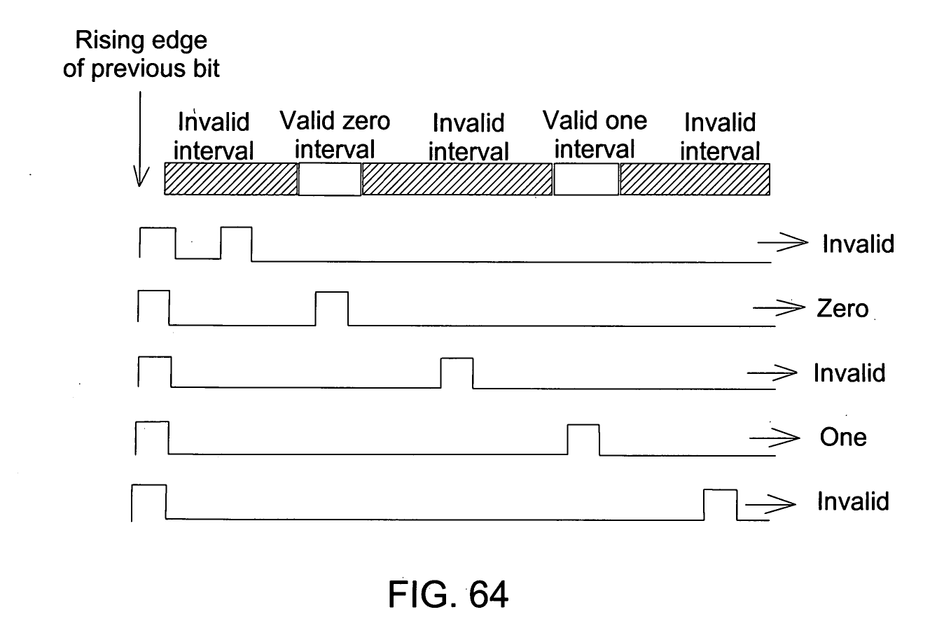

Actual programming is shown in conjunction with FIGS. 63 and 64. Programming and telemetry messages comprise many bits; however, the coil interface can only transmit one bit at a time. In addition, the signal is modulated to the resonant frequency of the coils, and must be transmitted in a relatively short period of time, and must provide detection of erroneous data.

A programming message is comprised of five parts FIG. 63(a). The start bit indicates the beginning of the message and is used to synchronize the timing of the rest of the message. The parameter number specifies which parameter (e.g., mode, pulse width, delay) is to be programmed. In the example, in FIG. 63(a) the number 10010000 specifies the pulse rate to be specified. The parameter value represents the value that the parameter should be set to. This value may be an index into a table of possible values; for example, the value 00101100 represents a pulse stimulus rate of 80 pulses/min. The access code is a fixed number based on the stimulus generator model which must be matched exactly for the message to succeed. It acts as a security mechanism against use of the wrong programmer, errors in the message, or spurious programming from environmental noise. It can also potentially allow more than one programmable implant in the patient. Finally, the parity field is the bitwise exclusive—OR of the parameter number and value fields. It is one of several error-detection mechanisms.

All of the bits are then encoded as a sequence of pulses of 0.35-ms duration FIG. 63(b). The start bit is a single pulse. The remaining bits are delayed from their previous bit according to their bit value. If the bit is a zero, the delay is short (1.0); if it is a one, the delay is long (2.2 ms). This technique of pulse position coding, makes detection of errors easier.

The serial pulse sequence is then amplitude modulated for transmission FIG. 63(c). The carrier frequency is the resonant frequency of the coils. This signal is transmitted from one set of coils to the other and then demodulated back into a pulse sequence FIG. 63(d).

FIG. 64 shows how each bit of the pulse sequence is decoded from the demodulated signal. As soon as each bit is received, a timer begins timing the delay to the next pulse. If the pulse occurs within a specific early interval, it is counted as a zero bit (FIG. 64(b)). If it otherwise occurs with a later interval, it is considered to be a one bit (FIG. 64(d)). Pulses that come too early, too late, or between the two intervals are considered to be errors and the entire message is discarded (FIG. 64(a, c, e)). Each bit begins the timing of the bit that follows it. The start bit is used only to time the first bit.

Telemetry data may be either analog or digital. Digital signals are first converted into a serial bit stream using an encoding such as shown in FIG. 64(b). The serial stream or the analog data is then frequency modulated for transmission.

An advantage of this and other encodings is that they provide multiple forms of error detection. The coils and receiver circuitry are tuned to the modulation frequency, eliminating noise at other frequencies. Pulse-position coding can detect errors by accepting pulses only within narrowly-intervals. The access code acts as a security key to prevent programming by spurious noise or other equipment. Finally, the parity field and other checksums provides a final verification that the message is valid. At any time, if an error is detected, the entire message is discarded.

Another more sophisticated type of pulse position modulation may be used to increase the bit transmission rate. In this, the position of a pulse within a frame is encoded into one of a finite number of values, e.g. 16. A special synchronizing bit is transmitted to signal the start of the frame. Typically, the frame contains a code which specifies the type or data contained in the remainder of the frame.

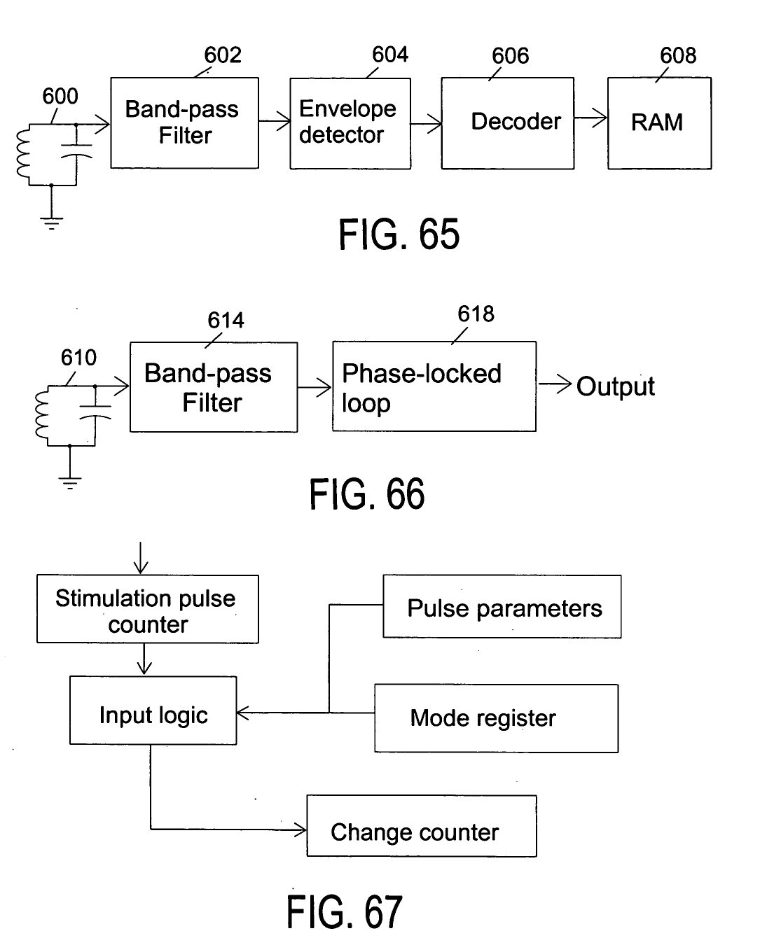

FIG. 65 shows a diagram of receiving and decoding circuitry for programming data. The IPG coil, in parallel with capacitor creates a tuned circuit for receiving data. The signal is band-pass filtered 602 and envelope detected 604 to create the pulsed signal in FIG. 63(d). After decoding, the parameter value is placed in a RAM at the location specified by the parameter number. The IPG can have two copies of the RAM-a permanent set and a temporary set-which makes it easy for the physician to set the IPG to a temporary configuration and later reprogram it back to the usual settings.

FIG. 66 shows the basic circuit used to receive telemetry data. Again, a coil and capacitor create a resonant circuit tuned to the carrier frequency. The signal is further band-pass filtered 614 and then frequency-demodulated using a phase-locked loop 618.

This embodiment also comprises an optional battery status test circuit. Shown in conjunction with FIG. 67, the charge delivered by the battery is estimated by keeping track of the number of pulses delivered by the IPG 391. An internal charge counter is updated during each test mode to read the total charge delivered. This information about battery status is read from the IPG 391 via telemetry.

### Combination Implantable Device Comprising Both a Stimulus-Receiver and a Programmable Implantable Pulse Generator (IPG)

In one embodiment, the implantable device may comprise both a stimulus-receiver and a programmable implantable pulse generator (IPG) in one device. Another embodiment of a similar device is disclosed in applicant's co-pending application Ser. No. 10/436,017. This embodiment also comprises predetermined/pre-packaged programs. Examples of several stimulation states were given in the previous sections, under “Programmer-less Implantable Pulse Generator (IPG)” and “Programmable Implantable Pulse Generator”. These predetermined/pre-packaged programs comprise unique combinations of pulse amplitude, pulse width, pulse frequency, ON-time and OFF-time.

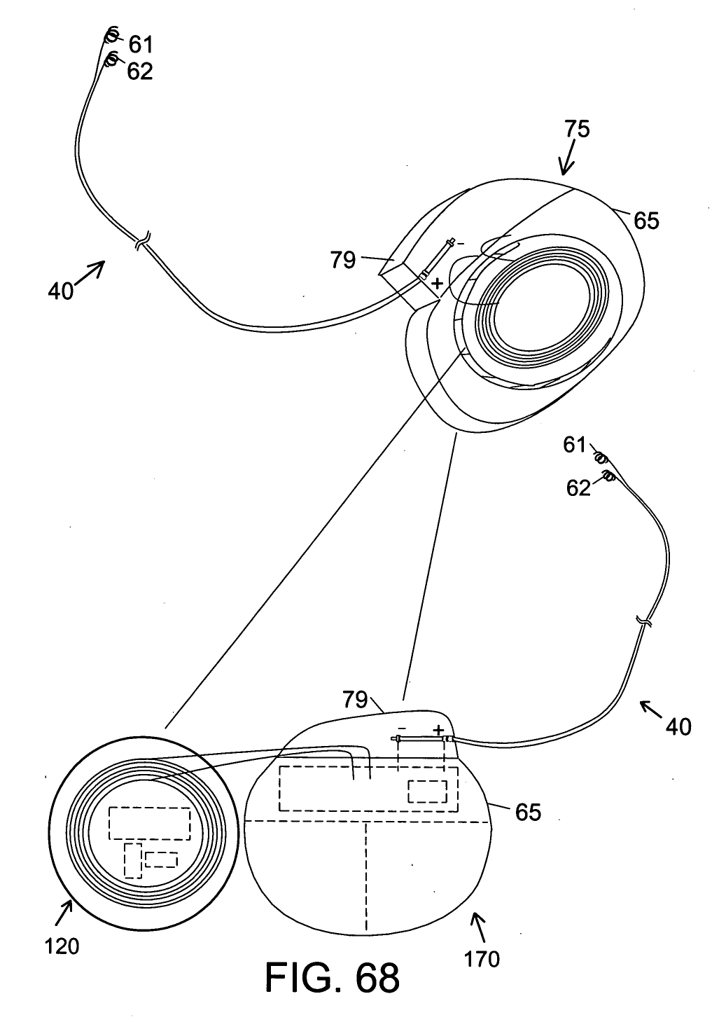

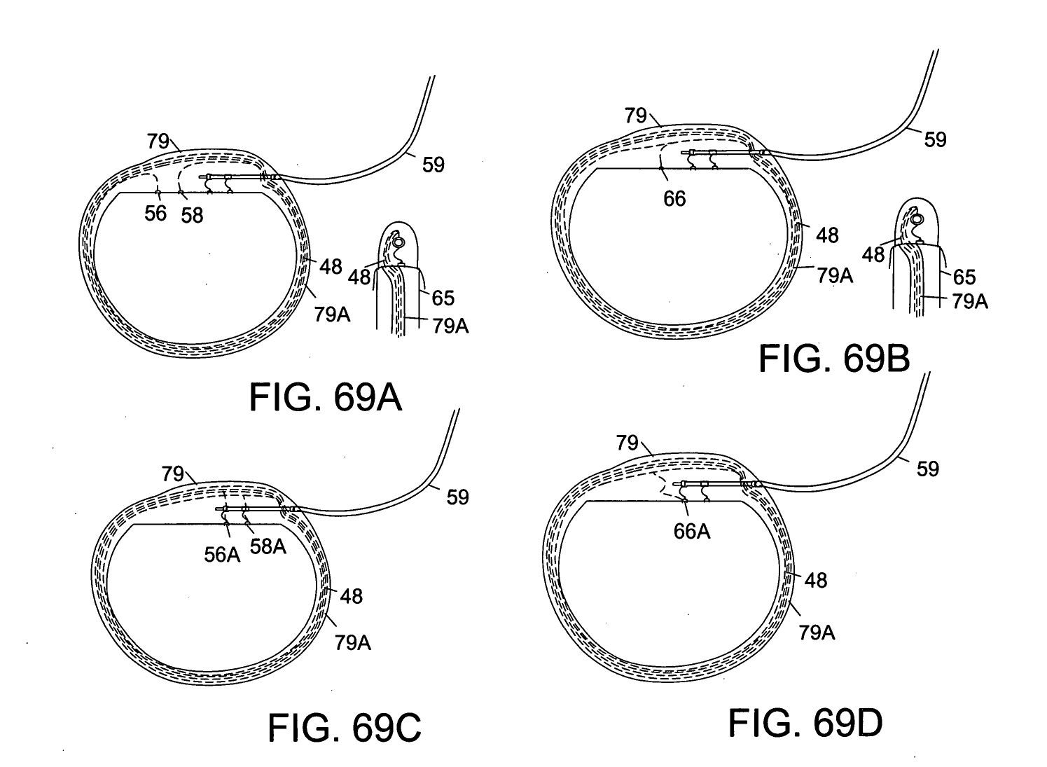

FIG. 68 shows a close up view of the packaging of the implanted stimulator 75 of this embodiment, showing the two subassemblies 120, 170. The two subassemblies are the stimulus-receiver module 120 and the battery operated pulse generator module 170. The electrical components of the stimulus-receiver module 120 may be substantially in the titanium case along with other circuitry, except for a coil. The coil may be outside the titanium case as shown in FIG. 68, or the coil 48C may be externalized at the header portion 79 of the implanted device, and may be wrapped around the titanium can. In this case, the coil is encased in the same material as the header 79, as shown in FIGS. 69A-69D. FIG. 69A depicts a bipolar configuration with two separate feed-throughs, 56, 58. FIG. 69B depicts a unipolar configuration with one separate feed-through 66. FIG. 69C, and 69D depict the same configuration except the feed-throughs are common with the feed-throughs 66A for the lead.

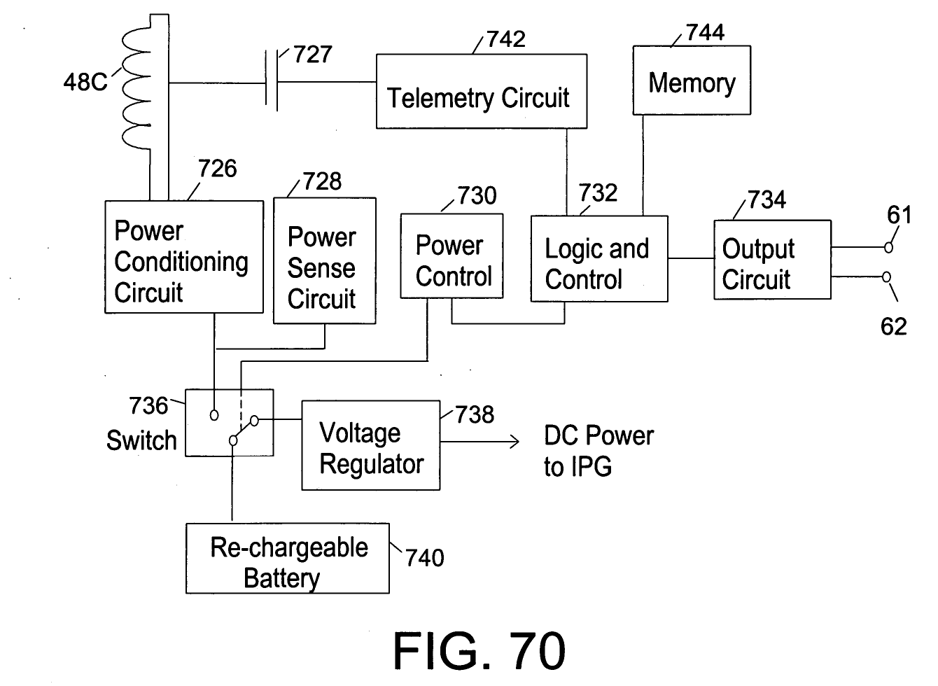

FIG. 70 is a simplified overall block diagram of the embodiment where the implanted stimulator 75 is a combination device, which may be used as a stimulus-receiver (SR) in conjunction with an external stimulator, or the same implanted device may be used as a traditional programmable implanted pulse generator (IPG). The coil 48C which is external to the titanium case may be used both as a secondary of a stimulus-receiver, or may also be used as the forward and back telemetry coil.

In this embodiment, as disclosed in FIG. 70, the IPG circuitry within the titanium case is used for all stimulation pulses whether the energy source is the internal battery 740 or an external power source. The external device serves as a source of energy, and as a programmer that sends telemetry to the IPG. For programming, the energy is sent as high frequency sine waves with superimposed telemetry wave driving the external coil 46C. Once received by the implanted coil 48C, the telemetry is passed through coupling capacitor 727 to the IPG's telemetry circuit 742. For pulse delivery using external power source, the stimulus-receiver portion will receive the energy coupled to the implanted coil 48C and, using the power conditioning circuit 726, rectify it to produce DC, filter and regulate the DC, and couple it to the IPG's voltage regulator 738 section so that the IPG can run from the externally supplied energy rather than the implanted battery 740.

The system provides a power sense circuit 728 that senses the presence of external power communicated with the power control 730 when adequate and stable power is available from an external source. The power control circuit controls a switch 736 that selects either battery power 740 or conditioned external power from 726. The logic and control section 732 and memory 744 includes the IPG's microcontroller, pre-programmed instructions, and stored chagneable parameters. Using input for the telemetry circuit 742 and power control 730, this section controls the output circuit 734 that generates the output pulses.

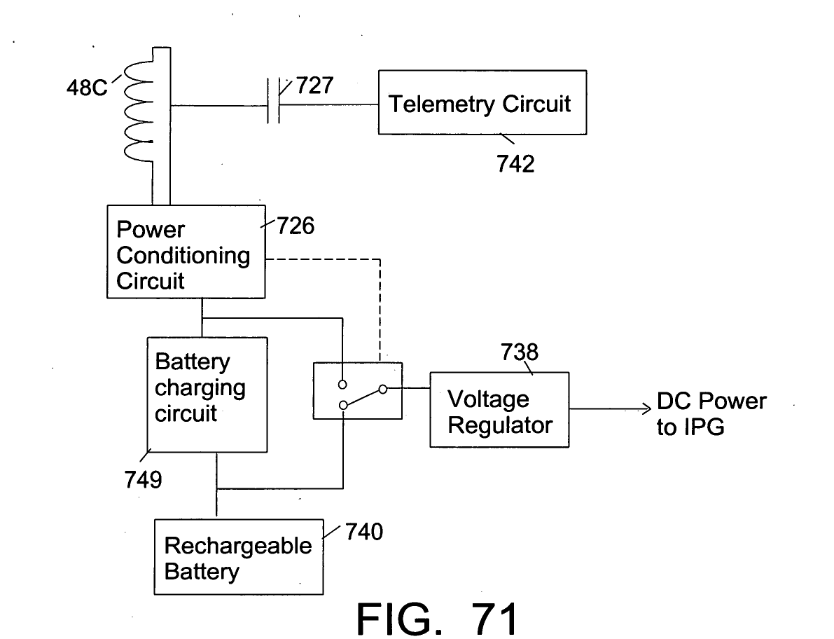

It will be clear to one skilled in the art that this embodiment of the invention can also be practiced with a rechargeable battery. This version is shown in conjunction with FIG. 71. The circuitry in the two versions are similar except for the battery charging circuitry 749. This circuit is energized when external power is available. It senses the charge state of the battery and provides appropriate charge current to safely recharge the battery without overcharging.

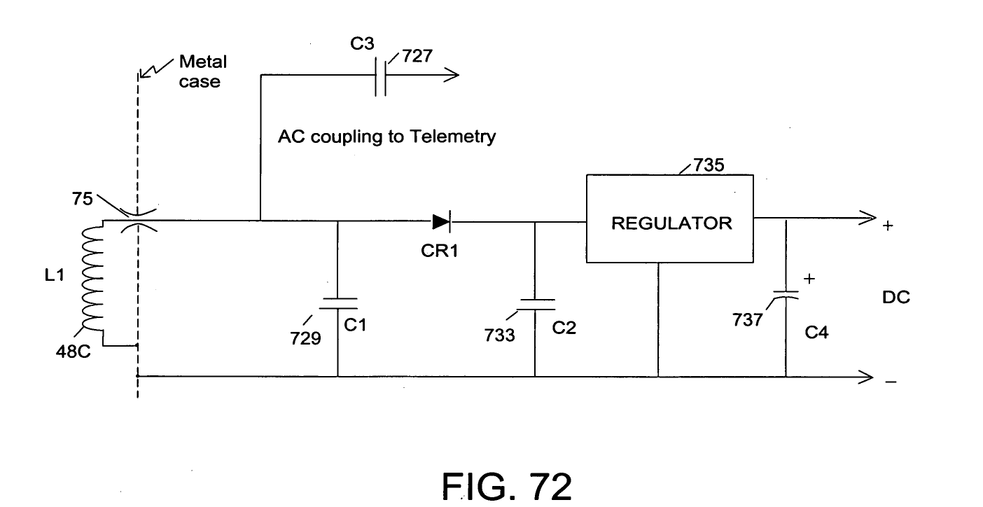

The stimulus-receiver portion of the circuitry is shown in conjunction with FIG. 72. Capacitor Cl (729) makes the combination of C1 and L1 sensitive to the resonant frequency and less sensitive to other frequencies, and energy from an external (primary) coil 46C is inductively transferred to the implanted unit via the secondary coil 48C. The AC signal is rectified to DC via diode 731, and filtered via capacitor 733. A regulator 735 sets the output voltage and limits it to a value just above the maximum IPG cell voltage. The output capacitor C4 (737), typically a tantalum capacitor with a value of 100 micro-Farads or greater, stores charge so that the circuit can supply the IPG with high values of current for a short time duration with minimal voltage change during a pulse while the current draw from the external source remains relatively constant. Also shown in conjunction with FIG. 72, a capacitor C3 (727) couples signals for forward and back telemetry.

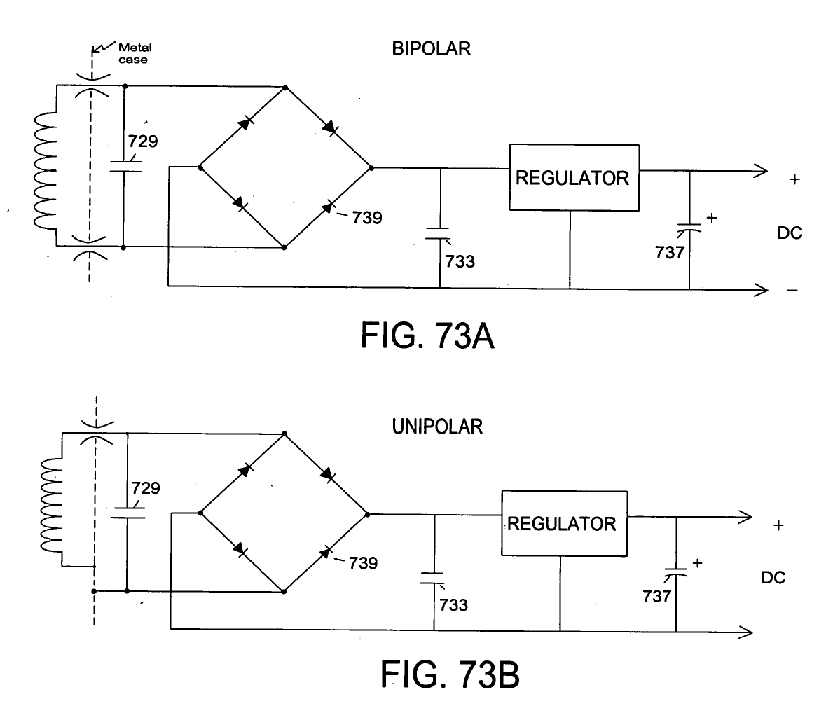

FIGS. 73A and 73B show alternate connection of the receiving coil. In FIG. 73A, each end of the coil is connected to the circuit through a hermetic feedthrough filter. In this instance, the DC output is floating with respect to the IPG's case. In FIG. 73B, one end of the coil is connected to the exterior of the IPG's case. The circuit is completed by connecting the capacitor 729 and bridge rectifier 739 to the interior of the IPG's case The advantage of this arrangement is that it requires one less hermetic feedthrough filter, thus reducing the cost and improving the reliability of the IPG. Hermetic feedthrough filters are expensive and a possible failure point. However, the case connection may complicit the output circuitry or limit its versatility. When using a bipolar electrode, care must be taken to prevent an unwanted return path for the pulse to the IPG's case. This is not a concern for unipolar pulses using a single conductor electrode because it relies on the IPG's case a return for the pulse current.

In the unipolar configuration, advantageously a bigger tissue area is stimulated since the difference between the tip (cathode) and case (anode) is larger. Stimulation using both configuration is considered within the scope of this invention.

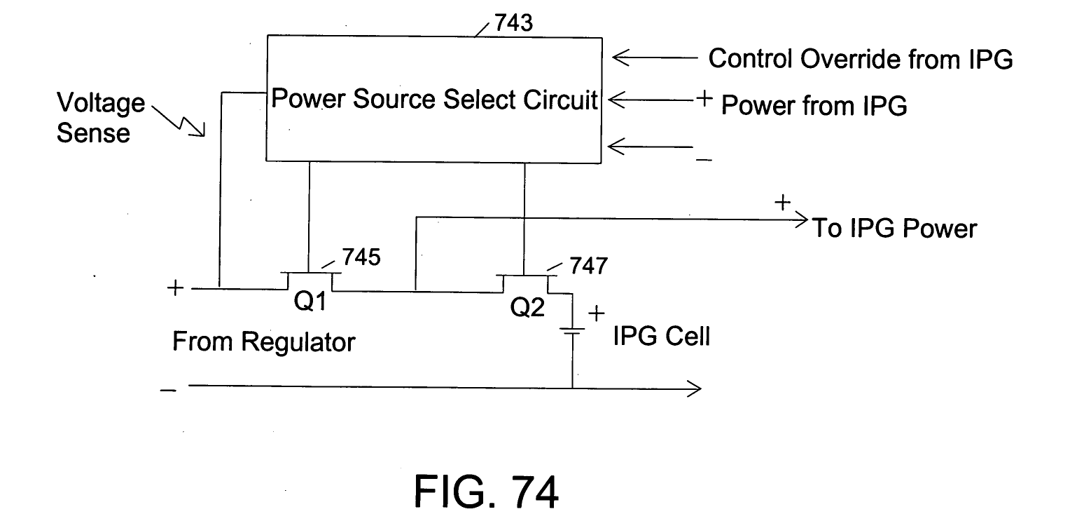

The power source select circuit is highlighted in conjunction with FIG. 74. In this embodiment, the IPG provides stimulation pulses according to the stimulation programs stored in the memory 744 of the implanted stimulator, with power being supplied by the implanted battery 740. When stimulation energy from an external stimulator is inductively received via secondary coil 48C, the power source select circuit (shown in block 743) switches power via transistor Q1 745 and transistor Q2 743. Transistor Q1 and Q2 are preferably low loss MOS transistor used as switches, even though other types of transistors may be used.

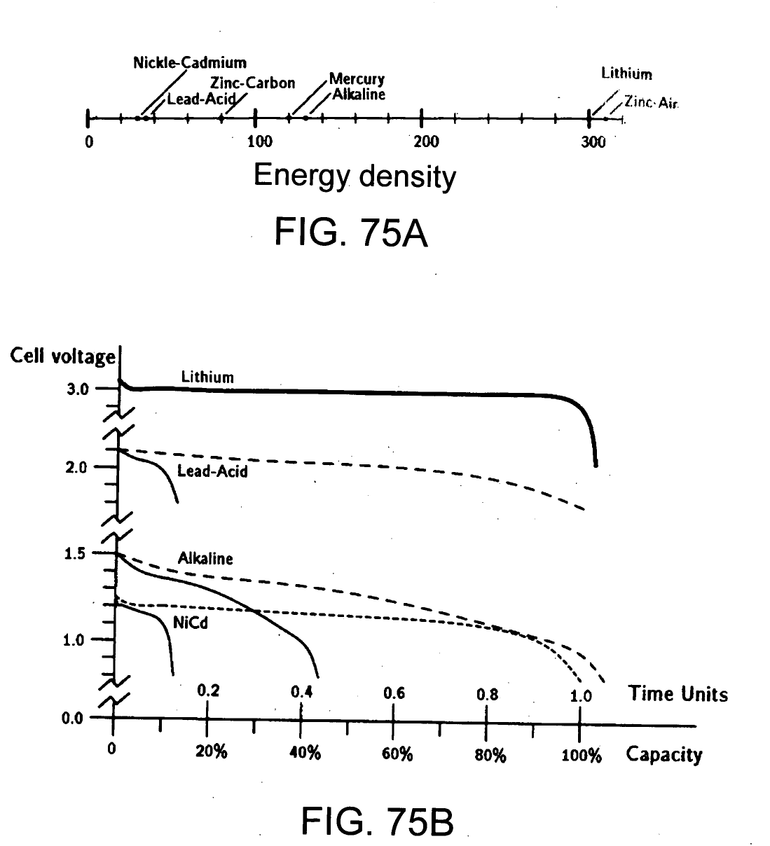

Implantable pulse generator (IPG) comprising a rechargable battery In one embodiment, an implantable pulse generator with rechargeable power source can be used. Because of the rapidity of the pulses required for modulating nerve tissue 54 with stimulating and/or blocking pulses, there is a real need for power sources that will provide an acceptable service life under conditions of continuous delivery of high frequency pulses. FIG. 75A shows a graph of the energy density of several commonly used battery technologies. Lithium batteries have by far the highest energy density of commonly available batteries. Also, a lithium battery maintains a nearly constant voltage during discharge. This is shown in conjunction with FIG. 75B, which is normalized to the performance of the lithium battery. Lithium-ion batteries also have a long cycle life, and no memory effect. However, Lithium-ion batteries are not as tolerant to overcharging and overdischarging. One of the most recent development in rechargable battery technology is the Lithium-ion polymer battery. Recently the major battery manufacturers (Sony, Panasonic, Sanyo) have announced plans for Lithium-ion polymer battery production.

In another embodiment, existing nerve stimulators and cardiac pacemakers can be modified to incorporate rechargeable batteries. Among the nerve stimulators that can be adopted with rechargeable batteries can for, example, be the vagus nerve stimulator manufactured by Cyberonics Inc. (Houston, Tex.). U.S. Pat. No. 4,702,254 (Zabara), U.S. Pat. No. 5,023,807 (Zabara), and U.S. Pat. No. 4,867,164 (Zabara) on Neurocybernetic Prostheses, which can be practiced with rechargeable power source as disclosed in the next section. These patents are incorporated herein by reference.

This embodiment also comprises predetermined/pre-packaged programs. Examples of several stimulation states were given in the previous sections, under “Programmer-less Implantable Pulse Generator (IPG)” and “Programmable Implantable Pulse Generator”. These pre-packaged/pre-determined programs comprise unique combinations of pulse amplitude, pulse width, pulse frequency, ON-time and OFF-time. Additionally, predetermined programs comprising blocking pulses may also be stored in the memory of the pulse generator.

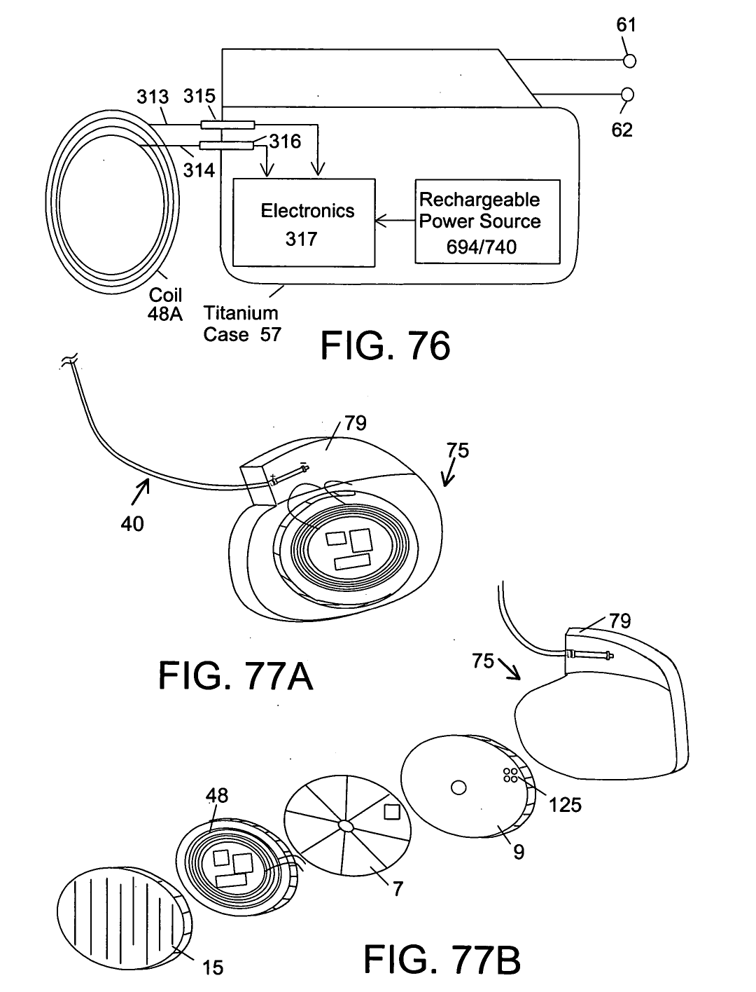

As shown in conjunction with FIG. 76, the coil is externalized from the titanium case 57. The RF pulses transmitted via coil 46 and received via subcutaneous coil 48A are rectified via a diode bridge. These DC pulses are processed and the resulting current applied to recharge the battery 694/740 in the implanted pulse generator. In one embodiment the coil 48C may be externalized at the header portion 79 of the implanted device, and may be wrapped around the titanium can, as was previously shown in FIGS. 59A-D.

In one embodiment, the coil may also be positioned on the titanium case as shown in conjunction with FIGS. 77A and 77B. FIG. 77A shows a diagram of the finished implantable stimulator 391 R of one embodiment. FIG. 77B shows the pulse generator with some of the components used in assembly in an exploded view. These components include a coil cover 15, the secondary coil 48 and associated components, a magnetic shield 7, and a coil assembly carrier 19. The coil assembly carrier 9 has at least one positioning detail 125 located between the coil assembly and the feed through for positioning the electrical connection. The positioning detail 125 secures the electrical connection.

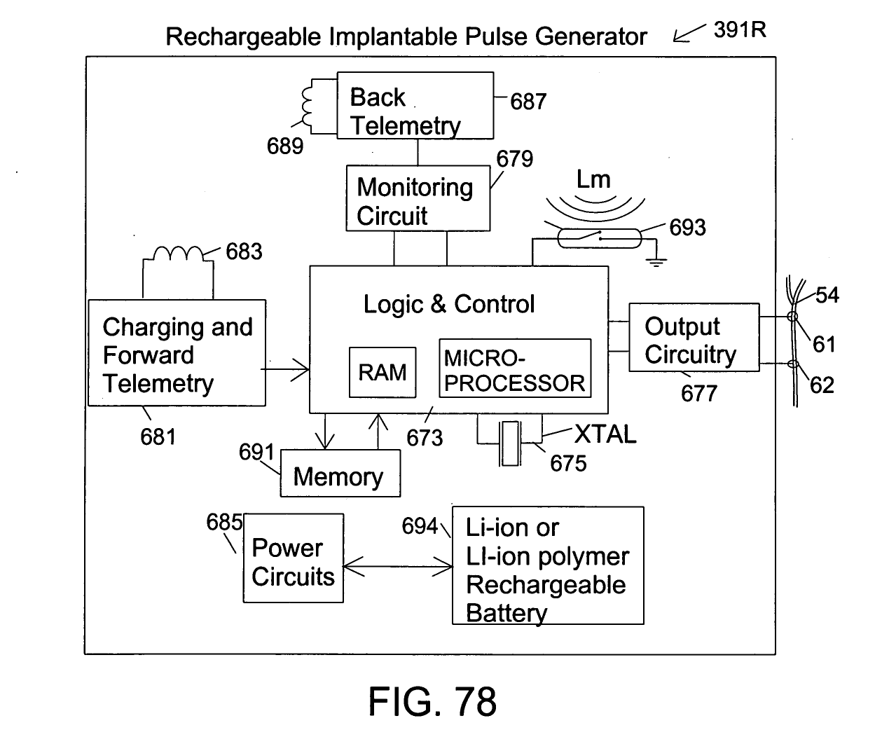

A schematic diagram of the implanted pulse generator (IPG 391R), with re-chargeable battery 694, is shown in conjunction with FIG. 78. The IPG 391R includes logic and control circuitry 673 connected to memory circuitry 691. The operating program and stimulation parameters are typically stored within the memory 691 via forward telemetry. Stimulation pulses are provided to the nerve tissue 54 via output circuitry 677 controlled by the microcontroller.

The operating power for the IPG 391 R is derived from a rechargeable power source 694. The rechargeable power source 694 comprises a rechargeable lithium-ion or lithium-ion polymer battery. Recharging occurs inductively from an external charger to an implanted coil 48B underneath the skin 60. The rechargeable battery 694 may be recharged repeatedly as needed. Additionally, the IPG 391R is able to monitor and telemeter the status of its rechargable battery 691 each time a communication link is established with the external programmer 85.

Much of the circuitry included within the IPG 391R may be realized on a single application specific integrated circuit (ASIC). This allows the overall size of the IPG 391R to be quite small, and readily housed within a suitable hermetically-sealed case. The IPG case is preferably made from a titanium and is shaped in a rounded case.

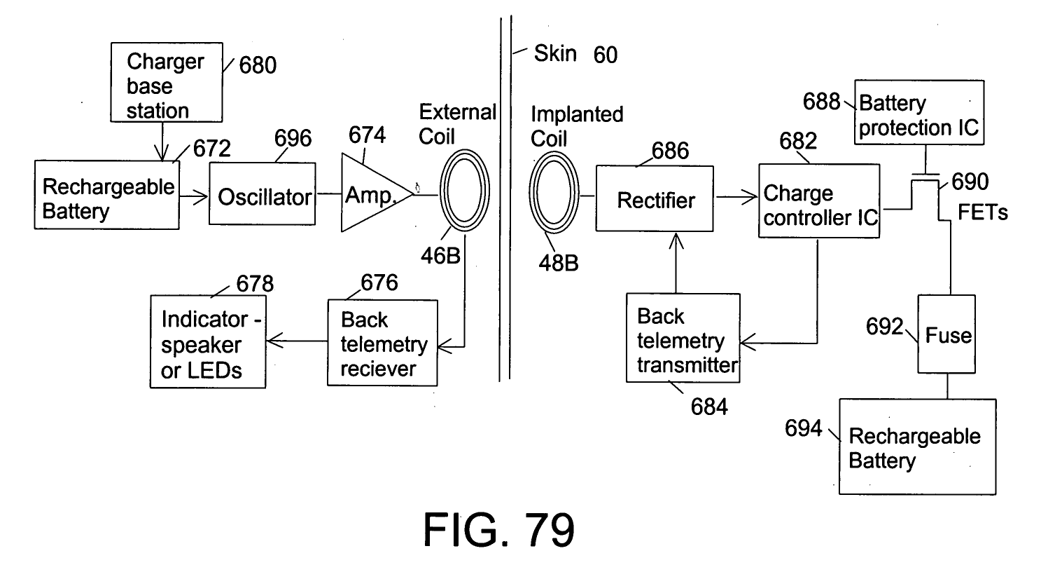

Shown in conjunction with FIG. 79 are the recharging elements of this embodiment. The re-charging system uses a portable external charger to couple energy into the power source of the IPG 391R. The DC-to-AC conversion circuitry 696 of the re-charger receives energy from a battery 672 in the re-charger. A charger base station 680 and conventional AC power line may also be used. The AC signals amplified via power amplifier 674 are inductively coupled between an external coil 46B and an implanted coil 48B located subcutaneously with the implanted pulse generator (IPG) 391R. The AC signal received via implanted coil 48B is rectified 686 to a DC signal which is used for recharging the rechargeable battery 694 of the IPG, through a charge controller IC 682. Additional circuitry within the IPG 391R includes, battery protection IC 688 which controls a FET switch 690 to make sure that the rechargeable battery 694 is charged at the proper rate, and is not overcharged. The battery protection IC 688 can be an off-the-shelf IC available from Motorola (part no. MC 33349N-3R1). This IC monitors the voltage and current of the implanted rechargeable battery 694 to ensure safe operation. If the battery voltage rises above a safe maximum voltage, the battery protection IC 688 opens charge enabling FET switches 690, and prevents further charging. A fuse 692 acts as an additional safeguard, and disconnects the battery 694 if the battery charging current exceeds a safe level. As also shown in FIG. 79, charge completion detection is achieved by a back-telemetry transmitter 684, which modulates the secondary load by changing the full-wave rectifier into a half-wave rectifier/voltage clamp. This modulation is in turn, sensed by the charger as a change in the coil voltage due to the change in the reflected impedance. When detected through a back telemetry receiver 676, either an audible alarm is generated or a LED is turned on.

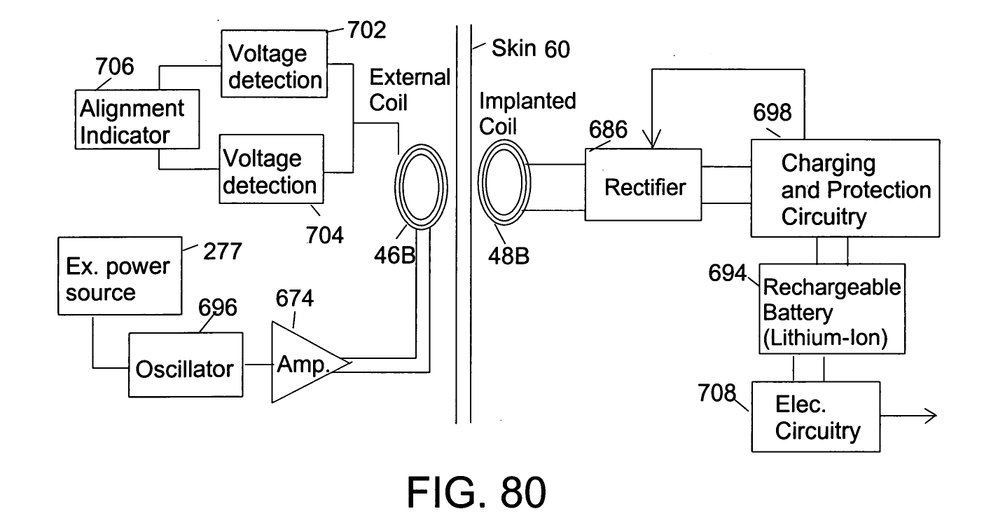

A simplified block diagram of charge completion and misalignment detection circuitry is shown in conjunction with FIG. 80. As shown, a switch regulator 686 operates as either a full-wave rectifier circuit or a half-wave rectifier circuit as controlled by a control signal (CS) generated by charging and protection circuitry 698. The energy induced in implanted coil 48B (from external coil 46B) passes through the switch rectifier 686 and charging and protection circuitry 698 to the implanted rechargeable battery 694. As the implanted battery 694 continues to be charged, the charging and protection circuitry 698 continuously monitors the charge current and battery voltage. When the charge current and battery voltage reach a predetermined level, the charging and protection circuitry 698 triggers a control signal. This control signal causes the switch rectifier 686 to switch to half-wave rectifier operation. When this change happens, the voltage sensed by voltage detector 702 causes the alignment indicator 706 to be activated. This indicator 706 may be an audible sound or a flashing LED type of indicator.

The indicator 706 may similarly be used as a misalignment indicator. In normal operation, when coils 46B (external) and 48B (implanted) are properly aligned, the voltage Vₛ sensed by voltage detector 704 is at a minimum level because maximum energy transfer is taking place. If and when the coils 46B and 48B become misaligned, then less than a maximum energy transfer occurs, and the voltage Vₛ sensed by detection circuit 704 increases significantly. If the voltage Vₛ reaches a predetermined level, alignment indicator 706 is activated via an audible speaker and/or LEDs for visual feedback. After adjustment, when an optimum energy transfer condition is established, causing Vₛ to decrease below the predetermined threshold level, the alignment indicator 706 is turned off.

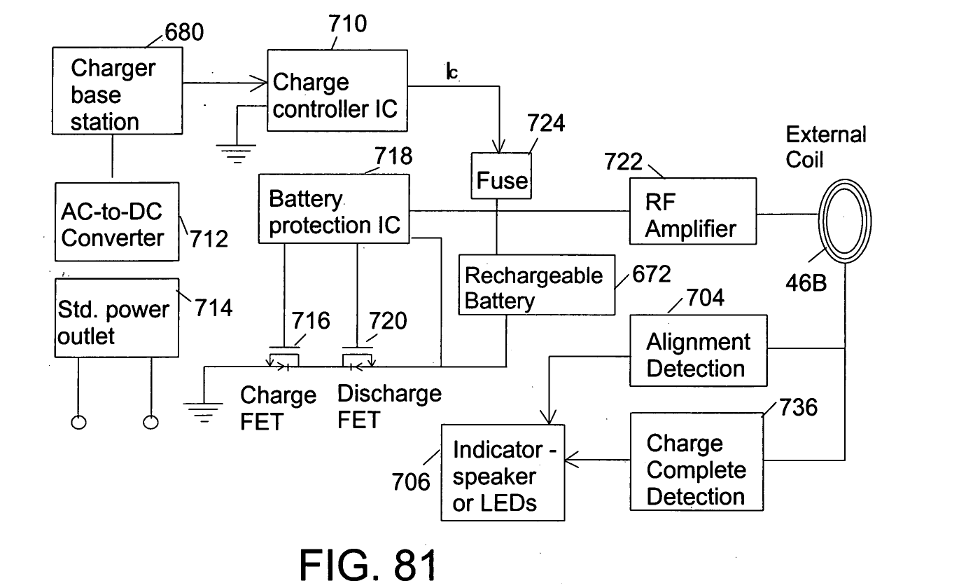

The elements of the external recharger are shown as a block diagram in conjunction with FIG. 81. In this disclosure, the words charger and recharger are used interchangeably. The charger base station 680 receives its energy from a standard power outlet 714, which is then converted to 5 volts DC by a AC-to-DC transformer 712. When the re-charger is placed in a charger base station 680, the re-chargeable battery 672 of the re-charger is fully recharged in a few hours and is able to recharge the battery 694 of the IPG 391R. If the battery 672 of the external re-charger falls below a prescribed limit of 2.5 volt DC, the battery 672 is trickle charged until the voltage is above the prescribed limit, and then at that point resumes a normal charging process.

As also shown in FIG. 81, a battery protection circuit 718 monitors the voltage condition, and disconnects the battery 672 through one of the FET switches 716, 720 if a fault occurs until a normal condition returns. A fuse 724 will disconnect the battery 672 should the charging or discharging current exceed a prescribed amount.

In summary, in the method of the current invention for neuromodulation of cranial nerve such as the vagus nerve(s), to provide adjunct therapy for involuntary movement disorders (including Parkinson's disease and epilepsy) be practiced with any of the several pulse generator systems disclosed including,

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a microstimulator;

e) a programmable implantable pulse generator;

f) a combination implantable device comprising both a stimulus-receiver and a programmable IPG; and

g) an IPG comprising a rechargeable battery.

Neuromodulation of vagus nerve(s) with any of these systems is considered within the scope of this invention.

### Remote Communications Module

In one embodiment, the external stimulator and/or the programmer has a telecommunications module, as described in a co-pending application, and summarized here for reader convenience. The telecommunications module has two-way communications capabilities.

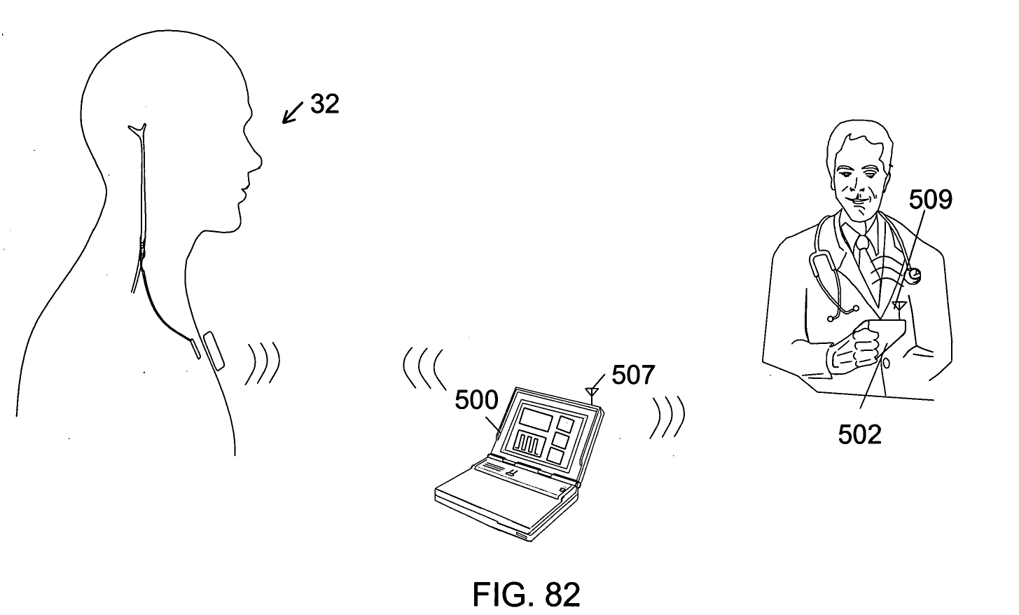

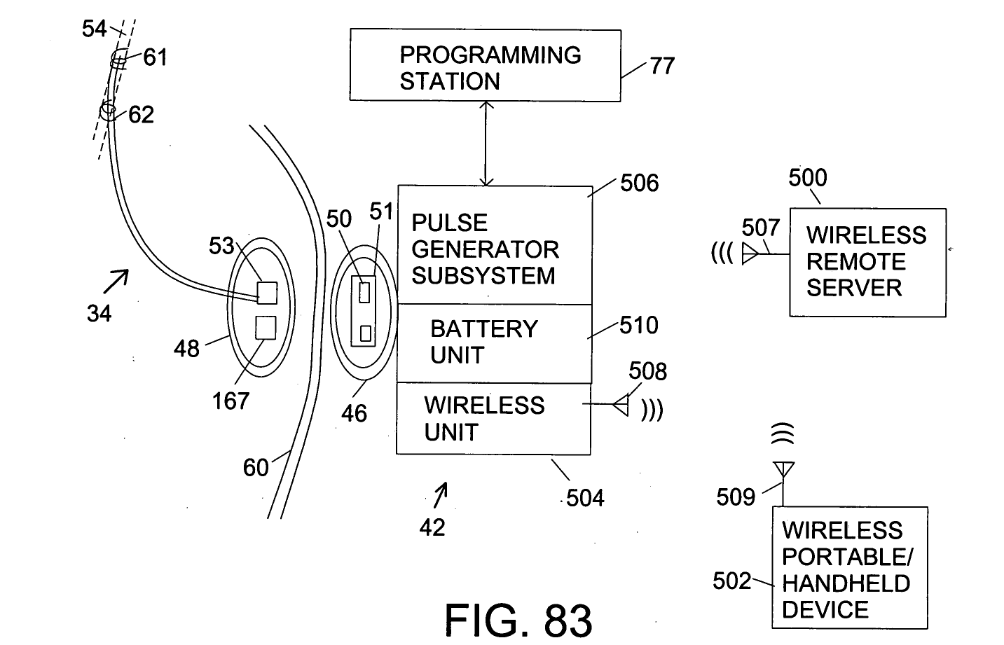

FIGS. 82 and 83 depict communication between an external stimulator 42 and a remote hand-held computer 502. A desktop or laptop computer can be a server 500 which is situated remotely, perhaps at a physician's office or a hospital. The stimulation parameter data can be viewed at this facility or reviewed remotely by medical personnel on a hand-held personal data assistant (PDA) 502, such as a “palm-pilot” from PALM corp. (Santa Clara, Calif.), a “Visor” from Handspring Corp. (Mountain view, Calif.) or on a personal computer (PC). The physician or appropriate medical personnel, is able to interrogate the external stimulator 42 device and know what the device is currently programmed to, as well as, get a graphical display of the pulse train. The wireless communication with the remote server 500 and hand-held PDA 502 would be supported in all geographical locations within and outside the United States (US) that provides cell phone voice and data communication service.

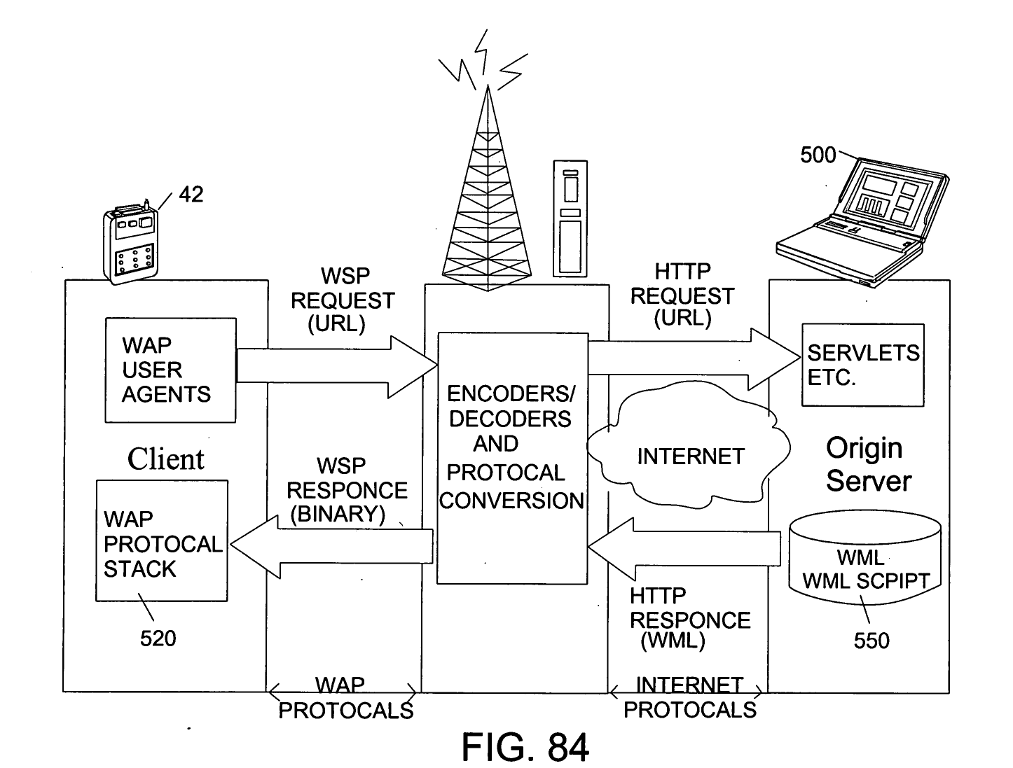

In one aspect of the invention, the telecommunications component can use Wireless Application Protocol (WAP). The Wireless Application Protocol (WAP), which is a set of communication protocols standardizing Internet access for wireless devices. While previously, manufacturers used different technologies to get Internet on hand-held devices, with WAP devices and services interoperate. WAP also promotes convergence of wireless data and the Internet. The WAP programming model is heavily based on the existing Internet programming model, and is shown schematically in FIG. 84. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops.

The key components of the WAP technology, as shown in FIG. 84, includes 1) Wireless Mark-up Language (WML) 550 which incorporates the concept of cards and decks, where a card is a single unit of interaction with the user. A service constitutes a number of cards collected in a deck. A card can be displayed on a small screen. WML supported Web pages reside on traditional Web servers. 2) WML Script which is a scripting language, enables application modules or applets to be dynamically transmitted to the client device and allows the user interaction with these applets. 3) Microbrowser, which is a lightweight application resident on the wireless terminal that controls the user interface and interprets the WML/WMLScript content. 4) A lightweight protocol stack 520 which minimizes bandwidth requirements, guaranteeing that a broad range of wireless networks can run WAP applications. The protocol stack of WAP can comprise a set of protocols for the transport (WTP), session (WSP), and security (WTLS) layers. WSP is binary encoded and able to support header caching, thereby economizing on bandwidth requirements. WSP also compensates for high latency by allowing requests and responses to be handled asynchronously, sending before receiving the response to an earlier request. For lost data segments, perhaps due to fading or lack of coverage, WTP only retransmits lost segments using selective retransmission, thereby compensating for a less stable connection in wireless. The above mentioned features are industry standards adopted for wireless applications and greater details have been publicized, and well known to those skilled in the art.

In this embodiment, two modes of communication are possible. In the first, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device to download these parameters.

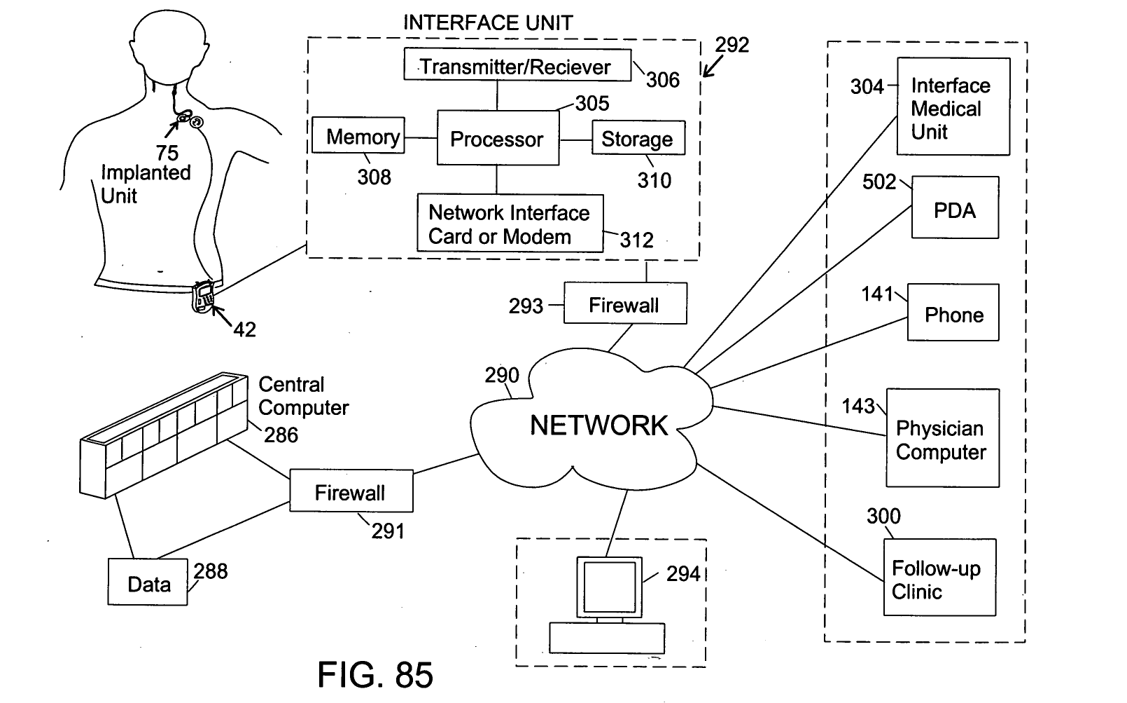

Shown in conjunction with FIG. 85, in one embodiment, the external stimulator 42 and/or the programmer 85 may also be networked to a central collaboration computer 286 as well as other devices such as a remote computer 294, PDA 502, phone 141, physician computer 143. The interface unit 292 in this embodiment communicates with the central collaborative network 290 via land-lines such as cable modem or wirelessly via the internet. A central computer 286 which has sufficient computing power and storage capability to collect and process large amounts of data, contains information regarding device history and serial number, and is in communication with the network 290. Communication over collaboration network 290 may be effected by way of a TCP/IP connection, particularly one using the internet, as well as a PSTN, DSL, cable modem, LAN, WAN or a direct dial-up connection.

The standard components of interface unit shown in block 292 are processor 305, storage 310, memory 308, transmitter/receiver 306, and a communication device such as network interface card or modem 312. In the preferred embodiment these components are embedded in the external stimulator 42 and can also be embedded in the programmer 85. These can be connected to the network 290 through appropriate security measures (Firewall) 293.

Another type of remote unit that may be accessed via central collaborative network 290 is remote computer 294. This remote computer 294 may be used by an appropriate attending physician to instruct or interact with interface unit 292, for example, instructing interface unit 292 to send instruction downloaded from central computer 286 to remote implanted unit.

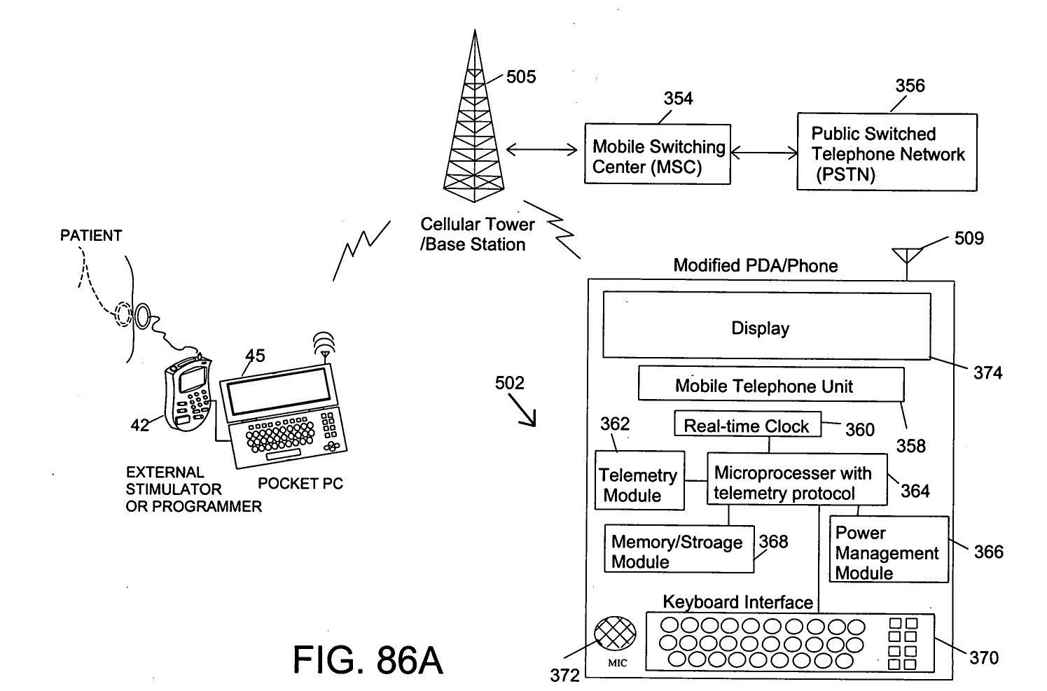

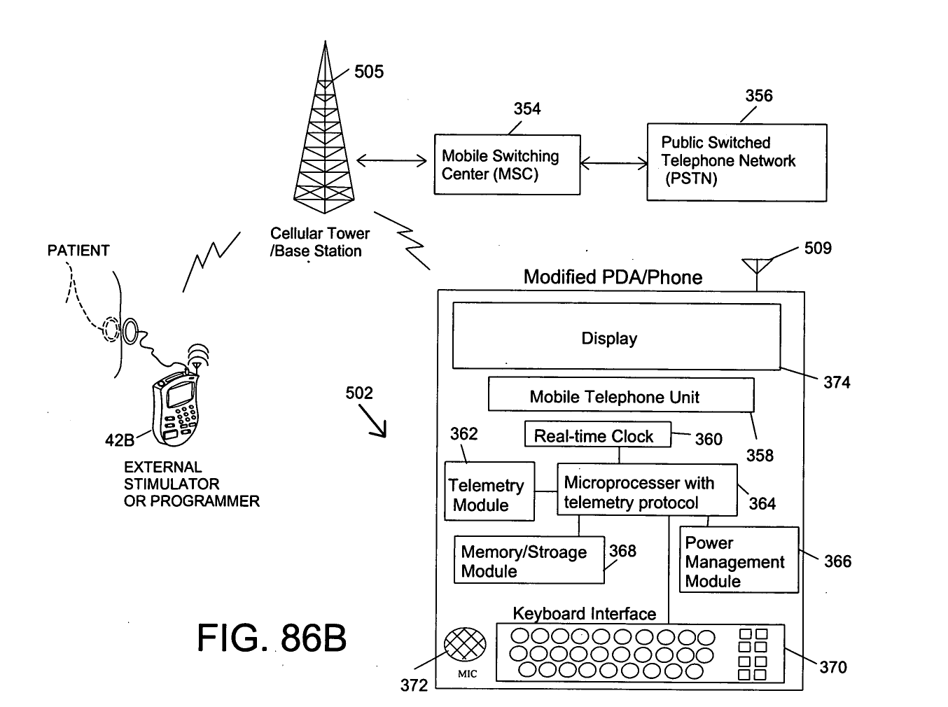

Shown in conjunction with FIGS. 86A and 86B the physician's remote communication's module is a Modified PDA/Phone 502 in this embodiment. The Modified PDA/Phone 502 is a microprocessor based device as shown in a simplified block diagram in FIGS. 76A and 76B. The PDA/Phone 502 is configured to accept PCM/CIA cards specially configured to fulfill the role of communication module 292 of the present invention. The Modified PDA/Phone 502 may operate under any of the useful software including Microsoft Window's based, Linux, Palm OS, Java OS, SYMBIAN, or the like.

The telemetry module 362 comprises an RF telemetry antenna 142 coupled to a telemetry transceiver and antenna driver circuit board which includes a telemetry transmitter and telemetry receiver. The telemetry transmitter and receiver are coupled to control circuitry and registers, operated under the control of microprocessor 364. Similarly, within stimulator a telemetry antenna 142 is coupled to a telemetry transceiver comprising RF telemetry transmitter and receiver circuit. This circuit is coupled to control circuitry and registers operated under the control of microcomputer circuit.

With reference to the telecommunications aspects of the invention, the communication and data exchange between Modified PDA/Phone 502 and external stimulator 42 operates on commercially available frequency bands. The 2.4-to-2.4853 GHZ bands or 5.15 and 5.825 GHz, are the two unlicensed areas of the spectrum, and set aside for industrial, scientific, and medical (ISM) uses. Most of the technology today including this invention, use either the 2.4 or 5 GHz radio bands and spread-spectrum technology.

The telecommunications technology, especially the wireless internet technology, which this invention utilizes in one embodiment, is constantly improving and evolving at a rapid pace, due to advances in RF and chip technology as well as software development. Therefore, one of the intents of this invention is to utilize “state of the art” technology available for data communication between Modified PDA/Phone 502 and external stimulator 42. The intent of this invention is to use 3G technology for wireless communication and data exchange, even though in some cases 2.5 G is being used currently.

For the system of the current invention, the use of any of the “3 G” technologies for communication for the Modified PDA/Phone 502, is considered within the scope of the invention. Further, it will be evident to one of ordinary skill in the art that as future 4 G systems, which will include new technologies such as improved modulation and smart antennas, can be easily incorporated into the system and method of current invention, and are also considered within the scope of the invention.

The present invention may be embodied in other specific forms without departing from the spirit or essential attributes thereof. It is therefore desired that the present embodiment be considered in all aspects as illustrative and not restrictive, reference being made to the appended claims rather than to the foregoing description to indicate the scope of the invention.

We claim:

1. A method of altering regional cerebral blood flow (rCBF) and/or altering neurochemicals in the brain for treating or alleviating the symptoms of depression, comprising the steps of providing complex and/or rectangular electrical pulses to a vagus nerve(s) its branches or parts thereof.

2. The method of claim 1, wherein said electrical pulses are provided, by a pulse generation means capable of providing complex and/or rectangular electrical pulses, and is one from a group comprising: i) an external stimulator used in conjunction with an implanted stimulus-receiver comprising a high value capacitor for storing electric charge; ii) a microstimulator; iii) a programmable implantable pulse generator (IPG); iv) a combination implantable device comprising both a programmable implantable pulse generator (IPG) and a stimulus-receiver; v) a programmable implantable pulse generator (IPG) having a rechargeable battery, and a lead in electrical connection with said pulse generation means, and further having at least one electrode adapted to be in contact with said vagus nerve(s) its branches or parts thereof.

3. The method of claim 1, wherein said complex electrical pulses comprises electrical pulses which are designed to be one of non-rectangular, multi-level pulses, biphasic, or pulses with varying amplitude during the pulse.

4. The method of claim 1, wherein the parameters of said electrical pulses are programmed to deliver intermittent electrical pulses for altering regional CBF and/or neurochemicals in the brain, without regard to synchronization or de-synchronization of patient's EEG.

5. The method of claim 2, wherein said pulse generation means further comprises at least two predetermined/pre-packaged programs stored in memory to control the variable component of said electric pulses, which comprises at least one of pulse amplitude, pulse width, pulse frequency, on-time and off-time time sequences.

6. The method of claim 2, wherein said pulse generation means may further comprise a telemetry means for remote interrogation and/or programming over a wide area network.

7. The method of claim 1, wherein said altering of regional CBF and/or altering neurochemicals in the brain by providing electrical pulses to said vagus nerve(s), can also be used for providing therapy or alleviating symptoms of epilepsy.

8. The method of claim 1, wherein said altering of neurochemicals comprises altering at least one of norepinephrine, serotonin, and epinephrine in the brain.

9. The method of claim 1, wherein said pulses further comprise pulse amplitude between 0.1 volt-15 volts; pulse width between 20 micro-seconds-5 milli-seconds; stimulation frequency between 5 Hz and 200 Hz, and blocking frequency between 0 and 750 Hz.

10. A method of providing complex and/or rectangular electrical pulses to a vagus nerve for treating or alleviating the symptoms of depression by altering regional CBF and/or neurochemicals in the brain, comprising the steps of:
providing pulse generation means capable of generating complex and rectangular electrical pulses, wherein said complex electrical pulses comprises at least one of multi-level pulses, biphasic pulses, non-rectangular pulses, or pulses with varying amplitude during the pulse;
providing a lead in electrical connection with said pulse generation means and with at least one electrode adapted to be in contact with said vagus nerve; and
activating said pulse generation means to provide said complex and/or rectangular electrical pulses to vagus nerve, its branches or part(s) thereof for altering regional CBF and/or neurochemicals in the brain.

11. The method of claim 10, wherein said pulse generation means for providing said electric pulses is one from a group comprising: i) an external stimulator used in conjunction with an implanted stimulus-receiver comprising a high value capacitor for storing electric charge; ii) a microstimulator; iii) a programmable implantable pulse generator (I PG); iv) a combination implantable device comprising both a programmable implantable pulse generator (IPG) and a stimulus-receiver; v) a programmable implantable pulse generator (IPG) having a rechargeable battery.

12. The method of claim 10, wherein the parameters of said electrical pulses are programmed to deliver intermittent electrical pulses for altering regional CBF and/or altering neurochemicals in the brain, without regard to sychronization or de-synchronization of patient's EEG.

13. The method of claim 10, wherein said method of providing complex and/or rectangular electrical pulses to vagus nerve for depression, is used in combination with providing repetitive transcranial magnetic stimulation (rTMS) therapy to the brain.

14. The method of claim 13, wherein said repetitive transcranial magnetic stimulation (rTMS) therapy provided to said patient, and said electrical pulses provided to said vagus nerve(s) may be provided in any sequence, any combination, or any time intervals.

15. The method of claim 10, wherein said method of providing complex and/or rectangular electrical pulses to vagus nerve to provide therapy for depression is used in combination with electroconvulsive therapy (ECT).

16. The method of claim 15, wherein said electroconvulsive therapy (ECT) provided to said patient, and said electrical pulses provided to said vagus nerve(s) may be provided in any sequence, any combination, or any interval of time.

17. The method of claim 10, wherein said pulse generation means may further comprise a telemetry means for remote interrogation and/or programming over a wide area network.

18. A method of stimulating and/or blocking a vagus nerve, its branches or parts thereof to alter regional cerebral blood flow (rCBF) and/or to alter neurochemicals in the brain with complex and/or rectangular electrical pulses, wherein said complex electrical pulses comprises at least one of multi-level pulses, biphasic pulses, non-rectangular pulses, or pulses with varying amplitude during the pulse, comprises the steps of:
providing pulse generation means for generating complex and/or rectangular electrical pulses, which is one from a group comprising: i) an external stimulator used in conjunction with an implanted stimulus-receiver comprising a high value capacitor for storing electric charge; ii) a microstimulator; iii) a programmable implantable pulse generator (IPG); iv) a combination implantable device comprising both a programmable implantable pulse generator (IPG) and a stimulus-receiver; v) a programmable implantable pulse generator (IPG) having a rechargeable battery;
providing a lead in electrical connection with said pulse generation means, and with at least one electrode adapted to be in contact with said vagus nerve; and
activating said pulse generation means to provide said rectangular and/or complex electrical pulses to selectively stimulate and/or block said vagus nerve, its branches or part(s) thereof.

19. The method of claim 18, wherein said method of stimulating and/or blocking said vagus nerve, its branches or parts thereof, is to provide therapy or alleviate symptoms of depression, wherein said depression further comprises bipolar depression, unipolar depression, severe depression, suicidal depression, psychotic depression, endogenous depression, treatment resistant depression, and melancholia.

20. The method of claim 18, wherein said pulse generation means further comprises at least two predetermined/pre-packaged programs stored in memory to control the variable component of said electrical pulses which comprise at least one of pulse amplitude, pulse width, pulse frequency, on-time and off-time time sequences.

21. The method of claim 18, wherein the parameters of said electrical pulses are programmed to deliver intermittent electrical pulses for altering regional CBF and/or neurochemicals in the brain without regard to sychronization or de-synchronization of patient's EEG.
